SEQUENCE LISTING (129 sequences)

SEQ ID NO: 1 - DNA, 32 bp, Artificial sequence
  Probe
  DNA (1)..(32): b = c, g ou t
aaacactctgatttcctcaaagtaaabgttct

SEQ ID NO: 2 - DNA, 30 bp, Artificial sequence
  Probe
ctaatgtattcaaggccaaacacctgcttg

SEQ ID NO: 3 - DNA, 24 bp, Artificial sequence
  Probe
cacgatcttgattattattccatg

SEQ ID NO: 4 - DNA, 29 bp, Artificial sequence
  Probe
gaggagtgtgactcgtcaaatatataccg

SEQ ID NO: 5 - DNA, 25 bp, Artificial sequence
  Probe
accacccactttgcattccaatgcc

SEQ ID NO: 6 - DNA, 19 bp, Artificial sequence
  Probe
ttctgcaaccaacgaccac

SEQ ID NO: 7 - DNA, 20 bp, Artificial sequence
  Probe
aacctttcccccaagatagg

SEQ ID NO: 8 - DNA, 24 bp, Artificial sequence
  Probe
gttgaacacatcagtgtagcgcgc

SEQ ID NO: 9 - DNA, 25 bp, Artificial sequence
  Probe
cctttccaggcaaggttgcaaactc

SEQ ID NO: 10 - DNA, 25 bp, Artificial sequence
  Probe
gatgcattttttcaagattacccaa

SEQ ID NO: 11 - DNA, 36 bp, Artificial sequence
  Probe
aatcaagtctgtttactttgtgtttagcaaatacaa

SEQ ID NO: 12 - DNA, 25 bp, Artificial sequence
  Probe
gcaagcactacaatctcactgagga

SEQ ID NO: 13 - DNA, 26 bp, Artificial sequence
  Probe
cacttgcagccaaaacccattgttaa

SEQ ID NO: 14 - DNA, 30 bp, Artificial sequence
  Probe
ccattcatatgcaagggtaataatttgcag

SEQ ID NO: 15 - DNA, 25 bp, Artificial sequence
  Probe
cattccaatgcccacaggcaaatta

SEQ ID NO: 16 - DNA, 27 bp, Artificial sequence
  Probe
caaatcacaaatcattgcaaacacatg

SEQ ID NO: 17 - DNA, 27 bp, Artificial sequence
  Probe
cctgggttggttctgtaactaatgacc

SEQ ID NO: 18 - DNA, 25 bp, Artificial sequence
  Probe
gaatcaccaaggttccaagcagagc

SEQ ID NO: 19 - DNA, 28 bp, Artificial sequence
  Probe
gttgccatgtaattcattcagtcattat

SEQ ID NO: 20 - DNA, 34 bp, Artificial sequence
  Probe
atcaagtctgtttactttgtgttcagcaaataca

SEQ ID NO: 21 - DNA, 25 bp, Artificial sequence
  Probe
caagtgcaacactcccaccaagcaa

SEQ ID NO: 22 - DNA, 26 bp, Artificial sequence
  Probe
acccacagcccaaagctcttgtcaag

SEQ ID NO: 23 - DNA, 25 bp, Artificial sequence
  Probe
caagtctgtttactttgtgttcagc

SEQ ID NO: 24 - DNA, 25 bp, Artificial sequence
  Probe
agtgcaacactcccaccaagcaaat

SEQ ID NO: 25 - DNA, 26 bp, Artificial sequence
  Probe
ccacagcccaaagctcttgtcaagca

SEQ ID NO: 26 - DNA, 28 bp, Artificial sequence
  Probe
aaacactctgatttcctcaaagtaaatg

SEQ ID NO: 27 - DNA, 25 bp, Artificial sequence
  Probe
gtattcaaggccaaacacctgcttg

SEQ ID NO: 28 - DNA, 29 bp, Artificial sequence
  Probe
cacgatcttgattattattccatgctaat

SEQ ID NO: 29 - DNA, 25 bp, Artificial sequence
  Probe
atgctcatcgcaaccacagcaaagc

SEQ ID NO: 30 - DNA, 28 bp, Artificial sequence
  Probe
ttgaggatggacagccctgcagccagac

SEQ ID NO: 31 - DNA, 24 bp, Artificial sequence
  Probe
tcgtcaagatctgtgcagaaggtt

SEQ ID NO: 32 - DNA, 25 bp, Artificial sequence
  Probe
cacttgcgttgtggcagcaaccaat

SEQ ID NO: 33 - DNA, 25 bp, Artificial sequence
  Probe
ccttgcagcatgtgacaatctcacc

SEQ ID NO: 34 - DNA, 26 bp, Artificial sequence
  Probe
cctgcagccagacaaacactaaagct

SEQ ID NO: 35 - DNA, 30 bp, Artificial sequence
  Probe
tacgcacaagcataacaacacacattcaca

SEQ ID NO: 36 - DNA, 27 bp, Artificial sequence
  Probe
acatcgccagttggtaccatgcaattc

SEQ ID NO: 37 - DNA, 33 bp, Artificial sequence
  Probe
attcgtttattatgaatcaccacgagaccaagc

SEQ ID NO: 38 - DNA, 23 bp, Artificial sequence
  Probe
  DNA (1)..(23): r = a ou g y = c ou t k = g ou t m = a ou c
rattcacamatygccakcacact

SEQ ID NO: 39 - DNA, 21 bp, Artificial sequence
  Probe
tgcgttgtggcagcaaccaat

SEQ ID NO: 40 - DNA, 25 bp, Artificial sequence
  Probe
cagactttccacggcaacaattagg

SEQ ID NO: 41 - DNA, 30 bp, Artificial sequence
  Probe
aactaaacattgctgcagtccgaattattc

SEQ ID NO: 42 - DNA, 25 bp, Artificial sequence
  Probe
ccttcctctaagtgataaggttcac

SEQ ID NO: 43 - DNA, 23 bp, Artificial sequence
  Probe
caccagactttccacggcaacgc

SEQ ID NO: 44 - DNA, 26 bp, Artificial sequence
  Probe
tcaggaactgaacactgctgcagtcc

SEQ ID NO: 45 - DNA, 27 bp, Artificial sequence
  Probe
cttctccttcctctaagtgttaaggtt

SEQ ID NO: 46 - DNA, 24 bp, Artificial sequence
  Probe
ccacggcaacgttcaggaactaaa

SEQ ID NO: 47 - DNA, 27 bp, Artificial sequence
  Probe
cattgctgcagtccgaattattcaccg

SEQ ID NO: 48 - DNA, 27 bp, Artificial sequence
  Probe
tctaagtgataaggttcaccagacttt

SEQ ID NO: 49 - DNA, 29 bp, Artificial sequence
  Probe
  DNA (1)..(29): y = c ou t
tgcacgacaacytaatgccacacagattc

SEQ ID NO: 50 - DNA, 26 bp, Artificial sequence
  Probe
  DNA (1)..(26): r = a ou g
cacccaaacatggcagaccagtacar

SEQ ID NO: 51 - DNA, 36 bp, Artificial sequence
  Probe
gaatttcacctctgacaatggaatacaaataccccc

SEQ ID NO: 52 - DNA, 33 bp, Artificial sequence
  Probe
aactgtccctcttaatcattaccttagtgcgca

SEQ ID NO: 53 - DNA, 30 bp, Artificial sequence
  Probe
ggacacaccacaaatccaactacgagcttt

SEQ ID NO: 54 - DNA, 40 bp, Artificial sequence
  Probe
ttaactgcaacaactttaatatacgctattggagctggaa

SEQ ID NO: 55 - DNA, 26 bp, Artificial sequence
  Probe
aatcactcaaagagcaaaggccgact

SEQ ID NO: 56 - DNA, 23 bp, Artificial sequence
  Probe
gacaacgactcactctaccaggc

SEQ ID NO: 57 - DNA, 23 bp, Artificial sequence
  Probe
ggacgggagtctcaccctctcag

SEQ ID NO: 58 - DNA, 25 bp, Artificial sequence
  Probe
gcaatcccaaacaactcgactcgtt

SEQ ID NO: 59 - DNA, 25 bp, Artificial sequence
  Probe
aacccaaactcacgaaagctcacag

SEQ ID NO: 60 - DNA, 25 bp, Artificial sequence
  Probe
ctgcgctcttccaaagggatcaacc

SEQ ID NO: 61 - DNA, 25 bp, Artificial sequence
  Probe
cgcccaagccacaagtggccgggga

SEQ ID NO: 62 - DNA, 24 bp, Artificial sequence
  Probe
gcaatcagaacccatcctagtcct

SEQ ID NO: 63 - DNA, 31 bp, Artificial sequence
  Probe
gcgcggaagatgttaccaagcaacgtcagct

SEQ ID NO: 64 - DNA, 25 bp, Artificial sequence
  Probe
catggcaagaaacacaacacacatt

SEQ ID NO: 65 - DNA, 24 bp, Artificial sequence
  Probe
agctctagcatttccacgggtatc

SEQ ID NO: 66 - DNA, 29 bp, Artificial sequence
  Probe
catgtaagagtgaaccaccaaataaattg

SEQ ID NO: 67 - DNA, 25 bp, Artificial sequence
  Probe
taagttgggtaggagcgcatgtatt

SEQ ID NO: 68 - DNA, 24 bp, Artificial sequence
  Probe
acaccccaattgcctcgtaggcag

SEQ ID NO: 69 - DNA, 29 bp, Artificial sequence
  Probe
ggagctgcaaccaaccaccaatttttcag

SEQ ID NO: 70 - DNA, 22 bp, Artificial sequence
  Probe
tcctcgtctgcgtgtcagggct

SEQ ID NO: 71 - DNA, 24 bp, Artificial sequence
  Probe
ccgcaaatgagttctgccaaggct

SEQ ID NO: 72 - DNA, 24 bp, Artificial sequence
  Probe
attcactcacccgtagacgagcta

SEQ ID NO: 73 - DNA, 24 bp, Artificial sequence
  Probe
gcaaccagacacgaaagaaacagt

SEQ ID NO: 74 - DNA, 25 bp, Artificial sequence
  Probe
ggacgaggaaccctcatccagattt

SEQ ID NO: 75 - DNA, 27 bp, Artificial sequence
  Probe
acaacatgagctcttcccgcttcactc

SEQ ID NO: 76 - DNA, 25 bp, Artificial sequence
  Probe
gcacgcatctctagactacaattcg

SEQ ID NO: 77 - DNA, 30 bp, Artificial sequence
  Probe
atgttccgcaaaaagcatccagcatacaaa

SEQ ID NO: 78 - DNA, 25 bp, Artificial sequence
  Probe
cagtccaagccacgacagagaatgt

SEQ ID NO: 79 - DNA, 25 bp, Artificial sequence
  Probe
cacaggcaacggcaagatggaacct

SEQ ID NO: 80 - DNA, 25 bp, Artificial sequence
  Probe
ctttcacaaacagacttagccctgg

SEQ ID NO: 81 - DNA, 25 bp, Artificial sequence
  Probe
gggttatcaccgtccatgacactgt

SEQ ID NO: 82 - DNA, 25 bp, Artificial sequence
  Probe
cgctacaacatccaaggcaagaacc

SEQ ID NO: 83 - DNA, 24 bp, Artificial sequence
  Probe
caccgccccgctttcgctggaata

SEQ ID NO: 84 - DNA, 25 bp, Artificial sequence
  Probe
gaaggtgatcattgctgcgtgcgac

SEQ ID NO: 85 - DNA, 28 bp, Artificial sequence
  Probe
gaaacatcacaccgaacaaatcacagcc

SEQ ID NO: 86 - DNA, 30 bp, Artificial sequence
  Probe
gcatttttaaaagattacccaatcctgtcg

SEQ ID NO: 87 - DNA, 26 bp, Artificial sequence
  Probe
gacaaggtcgtaaactcgttgagtgc

SEQ ID NO: 88 - DNA, 26 bp, Artificial sequence
  Probe
ttgcaataatctatccccatcacgat

SEQ ID NO: 89 - DNA, 27 bp, Artificial sequence
  Probe
cgtatagttaacggcctccaatctcta

SEQ ID NO: 90 - DNA, 27 bp, Artificial sequence
  Probe
gtcggcatggtttatggttaagactag

SEQ ID NO: 91 - DNA, 26 bp, Artificial sequence
  Probe
gatttctcataaggtgctgaaggagt

SEQ ID NO: 92 - DNA, 22 bp, Artificial sequence
  Probe
acatctacgcccctgctggcag

SEQ ID NO: 93 - DNA, 26 bp, Artificial sequence
  Probe
cgcgtcgagagattacaattcgtggc

SEQ ID NO: 94 - DNA, 26 bp, Artificial sequence
  Probe
cagtgattcgcttttccaagcggctt

SEQ ID NO: 95 - DNA, 25 bp, Artificial sequence
  Probe
caacgttcaggatctgaacactgcg

SEQ ID NO: 96 - DNA, 24 bp, Artificial sequence
  Probe
gcagtccgaattattcaccggatc

SEQ ID NO: 97 - DNA, 24 bp, Artificial sequence
  Probe
taaggttcactaaactttccactg

SEQ ID NO: 98 - DNA, 25 bp, Artificial sequence
  Probe
gcagtgcgaccagacacacagtgag

SEQ ID NO: 99 - DNA, 28 bp, Artificial sequence
  Probe
cagttcaaaaatgagttcaactggggac

SEQ ID NO: 100 - DNA, 27 bp, Artificial sequence
  Probe
gcagaagattgcaagcaagcacacgga

SEQ ID NO: 101 - DNA, 25 bp, Artificial sequence
  Probe
cttcatgcagagcagaagatcgcag

SEQ ID NO: 102 - DNA, 27 bp, Artificial sequence
  Probe
gcaagcacatgaagcagtgctaccaga

SEQ ID NO: 103 - DNA, 30 bp, Artificial sequence
  Probe
tgtcaaggacatgctcctggcaccaacaac

SEQ ID NO: 104 - DNA, 25 bp, Artificial sequence
  Probe
cgttcaggatctgaacactgcggca

SEQ ID NO: 105 - DNA, 25 bp, Artificial sequence
  Probe
gtccgaattattcaccggatcactc

SEQ ID NO: 106 - DNA, 24 bp, Artificial sequence
  Probe
ggttcactaaactttccactgcaa

SEQ ID NO: 107 - DNA, 26 bp, Artificial sequence
  Probe
cggagcagtgcgaccagacacacagt

SEQ ID NO: 108 - DNA, 29 bp, Artificial sequence
  Probe
gagcagttcaaaaatgagttcaatgggga

SEQ ID NO: 109 - DNA, 31 bp, Artificial sequence
  Probe
catgccaagcagaagattgcaagcaagcaca

SEQ ID NO: 110 - DNA, 24 bp, Artificial sequence
  Probe
agcagaagatcgcaggcaagcaca

SEQ ID NO: 111 - DNA, 29 bp, Artificial sequence
  Probe
tgaagcagtgctaccagacacacagagag

SEQ ID NO: 112 - DNA, 30 bp, Artificial sequence
  Probe
atgctcctggcaccaacaaccttcatgcag

SEQ ID NO: 113 - DNA, 25 bp, Artificial sequence
  Probe
agcagaagattgcaagcaagcacac

SEQ ID NO: 114 - DNA, 35 bp, Artificial sequence
  Probe
ggagcagtgcgaccagacacacagtgagcagttca

SEQ ID NO: 115 - DNA, 31 bp, Artificial sequence
  Probe
catgctcctggcactagcaaccttcatgcca

SEQ ID NO: 116 - DNA, 25 bp, Artificial sequence
  Probe
acatgctcctggcactagcaacctt

SEQ ID NO: 117 - DNA, 27 bp, Artificial sequence
  Probe
catgccaagcagaagattgcaagcaag

SEQ ID NO: 118 - DNA, 25 bp, Artificial sequence
  Probe
tttcgtcatcgttcttatgtcaagg

SEQ ID NO: 119 - DNA, 25 bp, Artificial sequence
  Probe
ggcaaacaggactgtcaccctcatt

SEQ ID NO: 120 - DNA, 29 bp, Artificial sequence
  Probe
agtgctcttttccaagaggcttacatctg

SEQ ID NO: 121 - DNA, 22 bp, Artificial sequence
  Probe
ccgtgcatggaagacgacagat

SEQ ID NO: 122 - DNA, 25 bp, Artificial sequence
  Probe
ggactgtcaccctcattagtgctct

SEQ ID NO: 123 - DNA, 26 bp, Artificial sequence
  Probe
tttccaagaggcttacatctgcaccc

SEQ ID NO: 124 - DNA, 25 bp, Artificial sequence
  Probe
catggaagacgacagatggcaaaca

SEQ ID NO: 125 - DNA, 25 bp, Artificial sequence
  Probe
ctgcacccccattggcaacgcatct

SEQ ID NO: 126 - DNA, 25 bp, Artificial sequence
  Probe
ccagactacaactcaaggctaccag

SEQ ID NO: 127 - DNA, 25 bp, Artificial sequence
  Probe
gtgctattttccaagaggcttacat

SEQ ID NO: 128 - DNA, 27 bp, Artificial sequence
  Probe
acgacaagaacagtgcgtttccccgaa

SEQ ID NO: 129 - DNA, 25 bp, Artificial sequence
  Probe
ggaccacgactgagcacgcaccttt

(19) United States
(12) Patent Application Publication
(10) Pub. No.: US 2022/0025470 A1
(43) Pub. Date: Jan. 27, 2022

(54) USE OF PROBES TO DETECT TOXIC ALGAE, DETECTION METHOD AND CORRESPONDING KITS

(71) Applicant: MICROBIA ENVIRONNEMENT, SAINT-JEAN LASEILLE (FR)

(72) Inventors: Delphine GUILLEBAULT, SAINT-JEAN-LASEILLE (FR); Elisa VILLA, PORT VENDRES (FR); Linda Karen MEDLIN CRAWFORD, PESSAC SUR DORDOGNE (FR)

(73) Assignee: MICROBIA ENVIRONNEMENT, SAINT-JEAN LASEILLE (FR)

(21) Appl. No.: 17/277,020

(22) PCT Filed: Sep. 17, 2019
(86) PCT No.: PCT/EP2019/074910

§ 371 Date: Mar. 17, 2021

(30) Foreign Application Priority Data
Sep. 17, 2018 (FR) 18/58362

(51) Int. Cl. C12Q 1/6895

(52) U.S. Cl. CPC C12Q 1/6895

(57) ABSTRACT

The use of probes for the detection of active living cells of toxic algae that are optionally present in a marine, brackish or industrial environment, a method for the detection of active living cells of toxic algae optionally present in a marine environment, and corresponding kits for providing the probes used for detection.

The present invention relates to the use of probes for the detection of active living cells of toxic algae, a method for the detection of active living cells of toxic algae and corresponding kits.

Recreational waters and aquaculture production sites are frequently and increasingly affected by toxic algal blooms. Toxic algae blooms are a real threat to human health, economic activities and the environment because they produce highly harmful phycotoxins that contaminate the food chain, making seafood unfit for consumption. The presence of these phycotoxins also threatens ecosystems and economic and tourist activities.

In this context, risk anticipation is a determining factor for sustainable blue growth, i.e. for a sustainable aquaculture, maritime and tourism economy. Rapid, reliable and sensitive identification of the toxic algae in question is essential for effective monitoring and anticipation of blooms in the sectors of activity concerned.

However, the composition of these environmental waters of interest depends on biotic and abiotic factors (organisms present, various organic materials, chemical or biological pollution, etc.). So much so that the heterogeneity in terms of
diversity, i.e. variability in content of organisms and organic and inorganic matter); and
physiological state, e.g. a population of a genus of microalgae may consist of several different species and may contain all life stages of a cell,
   will have a direct and deleterious impact on the detection of the toxic algae in question.

Current methods for the detection of toxic algae are mainly based on microscopic analysis or the sandwich hybridization technique. However, these methods are time-consuming and tedious. Their detection limits range from 1,000 to 3,000 cells/L for the algae Chattonella, Fibrocapsa, Heterosigma, Olithodiscus, Nannochloropsis (Compositions and methods for detecting raphidophytes, J V Tyrrell, P R Bergquist, P L Bergquist, C A Scholin, U.S. Pat. No. 6,787,648), up to 12,500 cells/L of Alexandrium (Colorimetric detection of the toxic Dinoflagellate Alexandrium minutum using sandwich hybridization in a microtiter plate assay, Sonja Diercks, Linda K Medlin, Katja Metfies—Harmful Algae, Vol. 7, Issue 2, February 2008, Pages 137-145) but also 70 ng/μL RNA for the algae Gymnodinium, Prorocentrum, Lingulodinium, Prymnesium, Chrysochromulina and Pseudo-nitzschia, (Molecular probe sets for the detection of toxic algae for use in sandwich hybridization formats, Sonja Diercks, Katja Metfies, Linda K. Medlin, Katja Metfies—Harmful Algae, Vol. Medlin, Journal of Plankton Research, Vol. 30, Issue 4, 1 Apr. 2008, Pages 439-448).

The sandwich hybridization technique (SHA) is known and consists in the detection of a nucleic acid of a species to be detected thanks to the use of both a capture probe immobilized on a solid support and a signal probe, both of which are specific to the nucleic acid of the species to be detected. The presence of the nucleic acid to be detected leads to the formation of a complex consisting of the capture probe, the nucleic acid of the species to be detected and the signal probe. Various means of detection can then be used to reveal the presence of the complex.

There is therefore a need for sensitive and reliable rapid techniques to detect the presence of toxic algae in various aquatic environments.

One of the goals of the invention is therefore to provide reliable, sensitive and rapid tools for the detection of active living cells of toxic algae, which effectively overcome and overcome the variability of environmental waters of interest.

A first aspect of the invention concerns the use of nucleotide probes for the implementation of a method for the detection of active living cells of toxic algae.

A second aspect of the invention concerns pairs of probes for the detection of active living cells of toxic algae.

A third aspect of the invention concerns probes for the detection of active living cells of toxic algae.

A fourth aspect of the invention relates to a method for detecting active living cells of toxic algae.

A fifth aspect of the invention concerns kits for the detection of active living cells of toxic algae.

A sixth aspect of the invention concerns devices for the detection of active living cells of toxic algae.

The present invention is based on the use of particular probes and the implementation of the sandwich hybridization technique in order to specifically detect and quantify the nucleic acids of active living cells of toxic algae optionally present in a marine, brackish or industrial environment at very low detection and quantification thresholds and in a time of less than 1 hour. Thus, the present invention provides a means of early warning making it optional to anticipate toxic algal blooms.

The novelty and inventiveness of the invention lies in the development of probes capable of recognizing and hybridizing in a very sensitive manner the ribosomal nucleic acids (rRNA or rDNA) of the large or small subunits of the targeted active toxic algae in less than one hour. By targeting the ribosomal nucleic acids, only active living cells of toxic algae can be detected,

i.e. algae capable of growing and proliferating, representing a major potential toxic risk. Thus, the present invention makes it optional to detect active living cells of toxic algae. Thanks to one or more calibration curve(s), it is then optional to estimate the number of active living cells of toxic algae present in a sample by taking into account the growth phase and the type of cell sought (Taylor et al., Harmful Algae 37 (2014) 17-27; Yuji Tanaka and Makoto Tsuneoka (2018), Control of Ribosomal RNA Transcription by Nutrients).

Toxic algae are algae that produce phycotoxins and cause food poisoning, paralysis, amnesia, skin irritation or fever. Toxic algae can be of different kinds. The probes used in the present invention make it optional to detect toxic algae of the genera Alexandrium, Dinophysis, Pseudo-nitzschia, Prorocentrum, Chattonella, Gymnodinium, Karenia, Lingulodinium and Heterosigma.

Active living cells are defined as cells that are capable of growing and dividing regardless of environmental conditions and that will be able to multiply and proliferate rapidly as soon as these environmental conditions are favourable, as opposed to dormant cells that have minimal cellular activity to protect themselves from environmental conditions unfavourable to their development, or senescent cells that have begun a method of cell death and that see their cellular and genetic material degraded.

According to the present invention, the terms “toxic algae” and “toxic micro-algae” are used interchangeably.

Threshold of Detection” or “Limit of Detection (LOD)” means the smallest amount of toxic algal RNA that can be detected by implementing the present invention. It is determined from an absorbance measurement at 450 nm or 630 nm made on an analytical blank from which the standard deviation of the signal is calculated, and corresponds to the concentration of toxic algal RNA which produces a signal whose intensity is equal to 3 times that of the standard deviation of the analytical blank. In other words, it is the value below which the toxic algal RNA is considered undetected. (ACS (1980) Guidelines for Data Acquisition and Data Quality Evaluation in Environmental Chemistry, Analytical chemistry, 52, 14, 2242-2249).

Threshold of Quantification” or “Limit of Quantification (LOQ)” means the smallest amount of toxic algal RNA that can be quantified by implementing the present invention. It is calculated by taking as the value of the signal, 10 times the value of the standard deviation of the analytical blank. In other words, this is the value below which it is not optional to determine the quantity of toxic algal RNA. (ACS (1980) Guidelines for Data Acquisition and Data Quality Evaluation in Environmental Chemistry, Analytical chemistry, 52, 14, 2242-2249).

Thus in a first aspect, the present invention concerns the use of at least one pair of specific probes of toxic algae for the implementation of a method for the detection of at least one toxic alga selected from the group consisting of Alexandrium, Dinophysis, Pseudo-nitzschia, Prorocentrum, Chattonella, Gymnodinium, Karenia, Lingulodinium and Heterosigma.

According to the present invention, the limit of detection of the algae listed above is less than or equal to 0.10 ng RNA per litre of sample and in particular is 0.01 ng RNA per litre of sample, which corresponds, depending on the type of algae, to a limit of detection of 100 to 500 active living cells per litre of sample (cells/L) and in particular less than 200 active living cells per litre of sample (cells/L). Furthermore, according to the present invention, the limit of quantification of the algae listed above is from 0.04 to 0.12 ng RNA per litre of sample depending on the type of algae.

Detection limit [ . . . ] less than or equal to 0.10 ng RNA per litre of sample” means the concentration corresponding to the minimum quantity of material measured according to the invention (i.e. ‘0.10 ng RNA’) in relation to a volume of one litre of raw natural sample taken in situ. Consequently, it is not the “chemical” concentration measured in a reaction volume following the implementation of the method of the invention to measure the quantity of target RNA in the sample.

Similarly, “detection limit [ . . . ] of 100 to 500 active living cells per litre of sample (cells/L)” means the minimum cell concentration estimated from standard curves that can be measured in a volume of one litre of raw natural sample taken in situ. Consequently, it is not the cell concentration measured in a reaction volume following the implementation of the method of the invention, which involves a treatment, including cell lysis, of the said raw sample taken in situ.

Since the present invention uses an absobance measurement read at 450 nm or 630 nm using a spectrophotometer, it should be noted that calibration curves can be made and used to pass from one unit to another (e.g. ng RNA per litre of sample) to a cell equivalent expressed in live active cells per litre of sample (cells/L).

Furthermore, it should be noted that for the purposes of the invention, it is also optional to use synthetic RNAs as an internal control and standardisation tool, said synthetic RNAs (or synthetic standard) being oligonucleotide sequences obtained by chemical synthesis. It is also understood that the microalgae cultures used are not axenic and the extraction of the total RNAs from the culture includes both the total RNAs of the target algae but also the total RNAs of the contaminants (e.g. bacteria) in the culture.

### Embodiment A

More particularly, the present invention concerns the use of at least one pair of probes specific to toxic algae of the genus Alexandrium for the implementation of a method for the detection of active living cells of toxic algae in a sample likely to contain at least one toxic alga of the genus Alexandrium, the sequences of said probes being chosen from x elements of one of the following sets:
(SEQ ID NO: 1, SEQ ID NO: 2 or SEQ ID NO: 3)
(SEQ ID NO: 4 and SEQ ID NO: 5)
(SEQ ID NO: 6 and SEQ ID NO: 7)
(SEQ ID NO: 8, SEQ ID NO: 9 or SEQ ID NO: 10)
(SEQ ID NO: 11, SEQ ID NO: 12 or SEQ ID NO: 13)
(SEQ ID NO: 14, SEQ ID NO: 15 or SEQ ID NO: 16)
(SEQ ID NO: 17, SEQ ID NO: 18 or SEQ ID NO: 19)
(SEQ ID NO: 20, SEQ ID NO: 21 or SEQ ID NO: 22)
(SEQ ID NO: 23, SEQ ID NO: 24 or SEQ ID NO: 25)
(SEQ ID NO: 26, SEQ ID NO: 27 or SEQ ID NO: 28)
   x being 2 or 3,
   or the sequences of said probes having at least 92% identity with said sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic alga of the genus Alexandrium optionally present in said sample to form a complex,
   the minimum detection threshold of the toxic algae of the genus Alexandrium being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

As mentioned above, a minimum detection limit for the toxic algae of the genus Alexandrium of less than 120 active living cells per litre of sample also corresponds to a minimum detection limit of 0.01 ng to 0.09 ng RNA per litre of sample.

For example, “a pair of probes, the sequences of said probes being selected from x elements of one of the following sets: (SEQ ID NO: 1, SEQ ID NO: 2 or SEQ ID NO: 3)” means the following pairs of probes:
SEQ ID NO: 1 and SEQ ID NO: 2
SEQ ID NO: 1 and SEQ ID NO: 3
SEQ ID NO: 2 and SEQ ID NO: 3.

Similarly, “a pair of probes, the sequences of said probes being selected from x elements of one of the following sets: (SEQ ID NO: 4 and SEQ ID NO: 5)”, the following sensor pair:
SEQ ID NO: 4 and SEQ ID NO: 5.

Furthermore, for the purposes of the invention, “less than 200 active living cells per litre of sample (cells/L)” means a minimum detection threshold lower than 195, 190, 185, 180, 175, 170, 165, 160, 155, 150, 145, 140, 135, 130, 125, 120, 115, 110, 100, 95, 90, 85, 80, 75, 70, 65, 60, 55, 50, 45, 40, 35, 30, 25, 20, 15, 10, 5, 4, 3, 2 or equal to 1 active live cell(s) per litre of sample (cells/L). It also means a minimum detection limit of 1 to 200, 1 to 50, 50 to 100, 100 to 150, 150 to 200, 50 to 200, 100 to 200 or 150 to 200 active live cell(s) per litre of sample (cells/L), in which case the minimum detection limit may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 active living cell(s) per litre of sample (cells/L).

Similarly, “less than or equal to 0.10 ng RNA per litre of sample (water, culture)” means a minimum detection limit of less than 0.09, 0.08, 0.07, 0.06, 0.05, 0.04, 0.03, 0.02 or 0.01 ng RNA per litre of sample. It also means a minimum detection limit of 0.01 to 0.10, 0.01 to 0.09, 0.05 to 0.10, 0.05 to 0.09 or 0.01 to 0.05 ng RNA per litre of sample, where the minimum detection limit may be 0.01, 0.02, 0.03, 0.04, 0.05, 0.06, 0.07, 0.08, 0.09 or 0.10 ng RNA per litre of sample.

The same reasoning may be applied to all aspects and embodiments of the present invention.

In this embodiment, the invention also concerns the use of at least one pair of probes specific to toxic algae of the genus Alexandrium for the implementation of a method for the detection of active living cells of toxic algae in a sample likely to contain at least one toxic algae of the genus Alexandrium, the sequences of said probes being chosen from x elements of one of the following sets:
(SEQ ID NO: 1, SEQ ID NO: 2 or SEQ ID NO: 3)
(SEQ ID NO: 4 and SEQ ID NO: 5)
(SEQ ID NO: 6 and SEQ ID NO: 7)
(SEQ ID NO: 8, SEQ ID NO: 9 or SEQ ID NO: 10)
(SEQ ID NO: 11, SEQ ID NO: 12 or SEQ ID NO: 13)
(SEQ ID NO: 14, SEQ ID NO: 15 or SEQ ID NO: 16)
(SEQ ID NO: 17, SEQ ID NO: 18 or SEQ ID NO: 19)
(SEQ ID NO: 20, SEQ ID NO: 21 or SEQ ID NO: 22),
(SEQ ID NO: 23, SEQ ID NO: 24 or SEQ ID NO: 25),
(SEQ ID NO: 26, SEQ ID NO: 27 or SEQ ID NO: 28)
   x being 2 or 3,
   or the sequences of said probes having at least 92% identity with the abovementioned sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic alga of the genus Alexandrium optionally present in said sample to form a complex,
   the minimum detection threshold of the toxic algae of the genus Alexandrium being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample,
   the duration of the implementation of the said detection method being less than one hour.

In this Embodiment, the invention also concerns the use of at least one pair of probes specific to toxic algae of the genus Alexandrium as described above in which the minimum detection threshold of the toxic algae of the genus Alexandrium is less than 120 active living cells per litre of sample (water, culture) (cells/L) or less than or equal to 0.10 ng RNA per litre of sample (water, culture) μL and the duration of the implementation of the said detection method is less than one hour.

According to this embodiment, the invention also concerns the use of at least one pair of probes specific to toxic algae of the genus Alexandrium as described above for the implementation of a method for detecting active living cells of toxic algae in a sample likely to contain at least one toxic algae of the genus Alexandrium, in which the sequences of the probes of the said pairs are as follows:
(SEQ ID NO: 1 and SEQ ID NO: 2), (SEQ ID NO: 1 and SEQ ID NO: 3), (SEQ ID NO: 2 and SEQ ID NO: 3)
(SEQ ID NO: 4 and SEQ ID NO: 5)
(SEQ ID NO: 6 and SEQ ID NO: 7)
(SEQ ID NO: 8 and SEQ ID NO: 9), (SEQ ID NO: 8 and SEQ ID NO: 10), (SEQ ID NO: 9 and SEQ ID NO: 10)
(SEQ ID NO: 11 and SEQ ID NO: 12), (SEQ ID NO: 11 and SEQ ID NO: 13), (SEQ ID NO: 12 and SEQ ID NO: 13)
(SEQ ID NO: 14 and SEQ ID NO: 15), (SEQ ID NO: 14 and SEQ ID NO: 16), (SEQ ID NO: 15 and SEQ ID NO: 16)
(SEQ ID NO: 17 and SEQ ID NO: 18), (SEQ ID NO: 17 and 19), (SEQ ID NO: 18 and SEQ ID NO: 19)
(SEQ ID NO: 20 and SEQ ID NO: 21), (SEQ ID NO: 20 and SEQ ID NO: 22), (SEQ ID NO: 21 and SEQ ID NO: 22), or
(SEQ ID NO: 23 and SEQ ID NO: 24), (SEQ ID NO: 23 and SEQ ID NO: 25), (SEQ ID NO: 24 and SEQ ID NO: 25)
(SEQ ID NO: 26 and SEQ ID NO: 27), (SEQ ID NO: 26 and SEQ ID NO: 28), (SEQ ID NO: 27 and SEQ ID NO: 28).

In addition to the detection of toxic algae of the genus Alexandrium, the present invention concerns, in addition to the use of at least one pair of probes specific to toxic algae of the genus Alexandrium, the use of at least one pair of probes specific to toxic algae selected from the group consisting of Dinophysis, Pseudo-nitzschia, Prorocentrum, Chattonella, Gymnodinium, Karenia, Lingulodinium and Heterosigma.

### Embodiment B

Thus, the invention also concerns the use as described above for the detection of active living cells of toxic algae of the genus Alexandrium (Embodiment A) comprising in addition the use of at least one pair of probes specific to toxic algae of the genus Dinophysis for the implementation of a method for the detection of active living cells of toxic algae in a sample likely to contain at least one toxic alga of the genus Alexandrium and/or Dinophysis, the sequences of said probes being chosen from x elements of one of the following sets:
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44 or SEQ ID NO: 45), or
(SEQ ID NO: 46, SEQ ID NO: 47 or SEQ ID NO: 48)
   x being 2 or 3,
   or the sequences of said probes having at least 92% identity with the abovementioned sequences SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic alga of the genus Dinophysis optionally present in said sample to form a complex,
   the minimum detection threshold of the toxic algae of the genus Dinophysis being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In a particular embodiment, the invention also concerns the use of at least one pair of probes specific to toxic algae of the genus Dinophysis as described above for the implementation of a method for the detection of active living cells of toxic algae in a sample likely to contain at least one toxic alga of the genus Dinophysis, wherein the sequences of the probes of said pairs are as follows:
(SEQ ID NO: 29 and SEQ ID NO: 30), (SEQ ID NO: 29 and SEQ ID NO: 31), (SEQ ID NO: 30 and SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40 and SEQ ID NO: 41), (SEQ ID NO: 40 and SEQ ID NO: 42), (SEQ ID NO: 41 and SEQ ID NO: 42)
(SEQ ID NO: 43 and SEQ ID NO: 44), (SEQ ID NO: 43 and SEQ ID NO: 45), (SEQ ID NO: 44 and SEQ ID NO: 45)
(SEQ ID NO: 46 and SEQ ID NO: 47), (SEQ ID NO: 46 and SEQ ID NO: 48), (SEQ ID NO: 47 and SEQ ID NO: 48.

As previously for the detection of Alexandrium according to embodiment A, and in a particular embodiment, a minimum detection threshold of the toxic algae of the genus Dinophysis lower than 200 active living cells per litre of sample (cells/L), also corresponds to a minimum detection threshold of 0.01 to 0.09 ng RNA per litre of sample.

In the same way, in a particular embodiment, the duration of the implementation of the said detection method is less than one hour.

Thus, embodiment B allows the detection of Alexandrium and Dinophysis thanks to the use of specific probes of toxic algae of the genus Alexandrium and Dinophysis.

### Embodiment C

In the same way, the invention also relates to one of the uses as described above according to embodiment A or according to embodiment B, further comprising the use of at least one pair of probes specific to toxic algae of the genus Pseudo-nitzschia for the implementation of a method for the detection of active living cells of toxic algae in a sample likely to contain at least one toxic alga of the genus Alexandrium and/or Pseudo-nitzschia, the sequences of said probes being selected from x elements of one of the following sets:
(SEQ ID NO: 49 and SEQ ID NO: 50)
(SEQ ID NO: 51 and SEQ ID NO: 52)
(SEQ ID NO: 53, SEQ ID NO: 54 or SEQ ID NO: 55)
(SEQ ID NO: 56, SEQ ID NO: 57, SEQ ID NO: 58)
(SEQ ID NO: 59, SEQ ID NO: 60, SEQ ID NO: 61)
   x being 2 or 3,
   or the sequences of said probes having at least 92% identity with the abovementioned sequences SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic alga of the genus Pseudo-nitzschia optionally present in said sample to form a complex,
   the minimum detection limit of the toxic algae of the genus Pseudo-nitzschia being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In a particular embodiment, the invention concerns the use of at least one pair of probes specific to toxic algae of the genus Pseudo-nitzschia as described above for the implementation of a method for the detection of active living cells of toxic algae in a sample likely to contain at least one toxic alga of the genus Pseudo-nitzschia in which the sequences of the probes of the said pairs are as follows:
(SEQ ID NO: 49 and SEQ ID NO: 50)
(SEQ ID NO: 51 and SEQ ID NO: 52)
(SEQ ID NO: 53 and SEQ ID NO: 54), (SEQ ID NO: 53 and SEQ ID NO: 55), (SEQ ID NO: 54 and SEQ ID NO: 55)
(SEQ ID NO: 56 and SEQ ID NO: 57), (SEQ ID NO: 56 and SEQ ID NO: 58), (SEQ ID NO: 57 and SEQ ID NO: 58)
(SEQ ID NO: 59 and SEQ ID NO: 60), (SEQ ID NO: 59 and SEQ ID NO: 61), (SEQ ID NO: 60 and SEQ ID NO: 61).

As previously for embodiments A and B, and in one particular embodiments, a minimum detection threshold of the toxic algae of the genus Pseudo-nitzschia of less than 200 active living cells per litre of sample (cells/L), also corresponds to a minimum detection threshold of 0.01 to 0.09 ng RNA per litre of sample and in particular 0.01 ng RNA per litre of sample.

In the same way, in a particular embodiment, the duration of the implementation of the said detection method is less than one hour.

Thus, embodiment C allows the detection:
C1: from Alexandrium and Pseudo-nitzschia (combined with A)
C2: of Alexandrium, Dinophysis and Pseudo-nitzschia (combined with B) thanks to the use of specific probes of toxic algae of the genus Alexandrium, Dinophysis and Pseudo-nitzschia.

### Embodiment D

In the same way, the invention also concerns one of the uses as described above according to embodiments A, B or C, comprising in addition the use of at least one pair of probes specific to toxic algae of the genus Prorocentrum for the implementation of a method for detecting active living cells of toxic algae in a sample likely to contain at least one toxic algae of the genus Alexandrium and/or Prorocentrum, the sequences of said probes being chosen from x elements of one of the following sets:
(SEQ ID NO: 62, SEQ ID NO: 63 or SEQ ID NO: 64)
(SEQ ID NO: 65, SEQ ID NO: 66 or SEQ ID NO: 67)
(SEQ ID NO: 68, SEQ ID NO: 69 or SEQ ID NO: 70)
(SEQ ID NO: 71, SEQ ID NO: 72 or SEQ ID NO: 73)
   x being 3,
   or the sequences of said probes having at least 92% identity with the abovementioned sequences SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72 or SEQ ID NO: 73,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence, said capture probe and said signal probe being capable of hybridizing with the ribosomal nucleic acid of a toxic alga of the genus Prorocentrum optionally present in said sample to form a complex,
   the minimum detection threshold of the toxic algae of the genus Prorocentrum being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In a particular embodiment, the invention concerns the use of at least one pair of probes specific to toxic algae of the genus Prorocentrum as described above for the implementation of a method for the detection of active living cells of toxic algae in a sample likely to contain at least one toxic algae of the genus Prorocentrum in which the sequences of the probes of said pairs are as follows:
(SEQ ID NO: 62 and SEQ ID NO: 63), (SEQ ID NO: 62 and SEQ ID NO: 64), (SEQ ID NO: 63 and SEQ ID NO: 64)
(SEQ ID NO: 65 and SEQ ID NO: 66), (SEQ ID NO: 65 and SEQ ID NO: 67), (SEQ ID NO: 66 and SEQ ID NO: 67)
(SEQ ID NO: 68 and SEQ ID NO: 69), (SEQ ID NO: 68 and SEQ ID NO: 70), (SEQ ID NO: 69 and SEQ ID NO: 70)
(SEQ ID NO: 71 and SEQ ID NO: 72), (SEQ ID NO: 71 and SEQ ID NO: 73), (SEQ ID NO: 72 and SEQ ID NO: 73)

As previously for embodiments A, B and C, and in one particular embodiment, a minimum detection limit for the toxic algae of the genus Prorocentrum of less than 200 active living cells per litre of sample (cells/L) also corresponds to a minimum detection limit of 0.01 ng to 0.09 ng RNA per litre of sample.

In the same way, in a particular embodiment, the duration of the implementation of the said detection method is less than one hour.

For example, embodiment D allows the detection of:
D1: from Alexandrium and Prorocentrum (in combination with A)
D2: from Alexandrium, Dinophysis and Prorocentrum (combined with B)
D3: from Alexandrium, Pseudo-nitzschia and Prorocentrum (combined with C1)
D4: from Alexandrium, Dinophysis, Pseudo-nitzschia and Prorocentrum (combined with C2)
   thanks to the use of specific probes of toxic algae of the genus Alexandrium, Dinophysis, Pseudo-nitzschia and Prorocentrum.

### Embodiment E

In the same way, the invention also concerns one of the uses as described above according to embodiments A, B, C or D, comprising in addition the use of at least one pair of probes specific to toxic algae of the genus Chattonella for the implementation of a method for detecting active living cells of toxic algae in a sample likely to contain at least one toxic algae of the genus Alexandrium and/or Chattonella, the sequences of said probes being chosen from x elements of one of the following sets:
(SEQ ID NO: 74, SEQ ID NO: 75 or SEQ ID NO: 76)
(SEQ ID NO: 77, SEQ ID NO: 78 or SEQ ID NO: 79)
(SEQ ID NO: 80, SEQ ID NO: 81 or SEQ ID NO: 82)
   x being 3,
   or the sequences of said probes having at least 92% identity with the abovementioned sequences SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81 or SEQ ID NO: 82,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence, said capture probe and said signal probe being capable of hybridizing with the ribosomal nucleic acid of a toxic algae of the genus Chattonella optionally present in said sample to form a complex,
   the minimum detection threshold of the toxic algae of the genus Chattonella being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In a particular embodiment, the invention concerns the use of at least one pair of probes specific to toxic algae of the genus Chattonella as described above for the implementation of a method for the detection of active living cells of algae in a sample likely to contain at least one toxic algae of the genus Chattonella in which the sequences of the probes of the said pairs are as follows:
(SEQ ID NO: 74 and SEQ ID NO: 75), (SEQ ID NO: 74 and SEQ ID NO: 76), (SEQ ID NO: 75 and SEQ ID NO: 76)
(SEQ ID NO: 77 and SEQ ID NO: 78), (SEQ ID NO: 77 and SEQ ID NO: 79), (SEQ ID NO: 78 and SEQ ID NO: 79)
(SEQ ID NO: 80 and SEQ ID NO: 81), (SEQ ID NO: 80 and SEQ ID NO: 82), (SEQ ID NO: 81 and SEQ ID NO: 82)

As previously for embodiments A, B, C and D, and in one particular embodiment, a minimum detection threshold of the toxic algae of the genus Chattonella of less than 200 active living cells per litre of sample (cells/L) also corresponds to a minimum detection threshold of between 0.01 ng and 0.09 ng RNA per litre of sample.

In the same way, in a particular embodiment, the duration of the implementation of the said detection method is less than one hour.

Thus, embodiment E allows the detection of:
E1: from Alexandrium and Chattonella (combined with A)
E2: from Alexandrium, Dinophysis and Chattonella (combined with B)
E3: of Alexandrium, Pseudo-nitzschia and Chattonella (combined with C1)
E4: of Alexandrium, Dinophysis, Pseudo-nitzschia and Chattonella (combined with C2)
E5: from Alexandrium, Prorocentrum and Chattonella (in combination with D1)
E6: from Alexandrium, Dinophysis, Prorocentrum and Chattonella (combined with D2)
E7: from Alexandrium, Pseudo-nitzschia, Prorocentrum and Chattonella (combined with D3)
E8: from Alexandrium, Dinophysis, Pseudo-nitzschia, Prorocentrum and Chattonella (combined with D4)
   thanks to the use of specific probes of toxic algae of the genus Alexandrium, Dinophysis, Pseudo-nitzschia, Prorocentrum and Chattonella.

### Embodiment F

In the same way, the invention also concerns one of the uses as described above according to the Embodiment A, B, C, D or E, comprising in addition the use of at least one pair of probes specific to toxic algae of the genus Gymnodinium for the implementation of a method for detecting active living cells of toxic algae in a sample likely to contain at least one toxic alga of the genus Alexandrium and/or Gymnodinium, the sequences of said probes being chosen from x elements of one of the following sets:
(SEQ ID NO: 83, SEQ ID NO: 84 or SEQ ID NO: 85)
(SEQ ID NO: 86, SEQ ID NO: 87 or SEQ ID NO: 88)
(SEQ ID NO: 89, SEQ ID NO: 90 or SEQ ID NO: 91)
(SEQ ID NO: 92, SEQ ID NO: 93 or SEQ ID NO: 94)
   x being 3,
   or the sequences of said probes having at least 92% identity with the abovementioned sequences SEQ ID NO: 83, SEQ ID NO: 84, SEQ ID NO: 85, SEQ ID NO: 6, SEQ ID NO: 87, SEQ ID NO: 88, SEQ ID NO: 89, SEQ ID NO: 90, SEQ ID NO: 91, SEQ ID NO: 92, SEQ ID NO: 93 or SEQ ID NO: 94,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic algae of the genus Gymnodinium optionally present in said sample to form a complex,
   the minimum detection threshold of the toxic algae of the genus Gymnodinium being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In a particular embodiment, the invention concerns the use of at least one pair of probes specific to toxic algae of the genus Gymnodinium as described above for the implementation of a method for the detection of active living cells of toxic algae in a sample likely to contain at least one toxic algae of the genus Gymnodinium in which the sequences of the probes of the said pairs are as follows:
(SEQ ID NO: 83 and SEQ ID NO: 84), (SEQ ID NO: 83 and SEQ ID NO: 85), (SEQ ID NO: 84 and SEQ ID NO: 85)
(SEQ ID NO: 86 and SEQ ID NO: 87), (SEQ ID NO: 86 and SEQ ID NO: 88), (SEQ ID NO: 87 and SEQ ID NO: 88)
(SEQ ID NO: 89 and SEQ ID NO: 90), (SEQ ID NO: 89 and SEQ ID NO: 91), (SEQ ID NO: 90 and SEQ ID NO: 91)
(SEQ ID NO: 92 and SEQ ID NO: 93), (SEQ ID NO: 92 and SEQ ID NO: 94), (SEQ ID NO: 93 and SEQ ID NO: 94).

As previously for embodiments A, B, C, D and E, and in one particular embodiment, a minimum detection threshold of the toxic algae of the genus Gymnodinium of less than 200 active living cells per litre of sample (cells/L) also corresponds to a minimum detection threshold of between 0.01 ng and 0.09 ng RNA per litre of sample.

In the same way, in a particular embodiment, the duration of the implementation of the said detection method is less than one hour.

For example, embodiment F allows the detection of:
F1: from Alexandrium and Gymnodinium (combined with A)
F2: from Alexandrium, Dinophysis and Gymnodinium (combined with B)
F3: of Alexandrium, Pseudo-nitzschia and Gymnodinium (combined with C1)
F4: of Alexandrium, Dinophysis, Pseudo-nitzschia and Gymnodinium (combined with C2)
F5: from Alexandrium, Prorocentrum and Gymnodinium (in combination with D1)
F6: from Alexandrium, Dinophysis, Prorocentrum and Gymnodinium (combined with D2)
F7: from Alexandrium, Pseudo-nitzschia, Prorocentrum and Gymnodinium (combined with D3)
F8: from Alexandrium, Dinophysis, Pseudo-nitzschia, Prorocentrum and Gymnodinium (combined with D4)
F9: from Alexandrium, Chattonella and Gymnodinium (combined with E1)
F10: from Alexandrium, Dinophysis, Chattonella and Gymnodinium (combined with E2)
F11: from Alexandrium, Pseudo-nitzschia, Chattonella and Gymnodinium (combined with E3)
F12: of Alexandrium, Dinophysis, Pseudo-nitzschia, Chattonella and Gymnodinium (combined with E4)
F13: from Alexandrium, Prorocentrum, Chattonella and Gymnodinium (combined with E5)
F14: from Alexandrium, Dinophysis, Prorocentrum, Chattonella and Gymnodinium (combined with E6)
F15: from Alexandrium, Pseudo-nitzschia, Prorocentrum, Chattonella and Gymnodinium (combined with E7)
F16: from Alexandrium, Dinophysis, Pseudo-nitzschia, Prorocentrum, Chattonella and Gymnodinium (combined with E8)
   thanks to the use of specific probes of toxic algae of the genus Alexandrium, Dinophysis, Pseudo-nitzschia, Prorocentrum, Chattonella and Gymnodinium.

### Embodiment G

In the same way, the invention also concerns one of the uses as described above according to s A, B, C, D, E or F, comprising in addition the use of at least one pair of probes specific to toxic algae of the genus Karenia for the implementation of a method for the detection of active living cells of toxic algae in a sample likely to contain at least one toxic alga of the genus Alexandrium and/or Karenia, the sequences of said probes being chosen from x elements of one of the following sets:
(SEQ ID NO: 95, SEQ ID NO: 96 or SEQ ID NO: 97)
(SEQ ID NO: 98, SEQ ID NO: 99 or SEQ ID NO: 100)
(SEQ ID NO: 101, SEQ ID NO: 102 or SEQ ID NO: 103)
(SEQ ID NO: 104, SEQ ID NO: 105 or SEQ ID NO: 106)
(SEQ ID NO: 107, SEQ ID NO: 108 or SEQ ID NO: 109)
(SEQ ID NO: 110, SEQ ID NO: 111 or SEQ ID NO: 112)
(SEQ ID NO: 113, SEQ ID NO: 114 or SEQ ID NO: 115)
(SEQ ID NO: 116, SEQ ID NO: 117 or SEQ ID NO: 118)
   x being 3,
   or the sequences of said probes having at least 92% identity with the abovementioned sequences SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic algae of the genus Karenia optionally present in said sample to form a complex,
   the minimum detection threshold of the toxic algae of the genus Karenia being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In a particular embodiment, the invention concerns the use of at least one pair of probes specific to toxic algae of the genus Karenia as described above for the implementation of a method for the detection of active living cells of toxic algae in a sample likely to contain at least one toxic algae of the genus Karenia in which the sequences of the probes of the said pairs are as follows:
(SEQ ID NO: 95 and SEQ ID NO: 96), (SEQ ID NO: 95 and SEQ ID NO: 97), (SEQ ID NO: 96 and SEQ ID NO: 97)
(SEQ ID NO: 98 and SEQ ID NO: 99), (SEQ ID NO: 98 and SEQ ID NO: 100), (SEQ ID NO: 99 and SEQ ID NO: 100)
(SEQ ID NO: 101 and SEQ ID NO: 102), (SEQ ID NO: 101 and SEQ ID NO: 103), (SEQ ID NO: 102 and SEQ ID NO: 103)
(SEQ ID NO: 104 and SEQ ID NO: 105), (SEQ ID NO: 104 and SEQ ID NO: 106), (SEQ ID NO: 105 and SEQ ID NO: 106)
(SEQ ID NO: 107 and SEQ ID NO: 108), (SEQ ID NO: 107 and SEQ ID NO: 109), (SEQ ID NO: 108 and SEQ ID NO: 109)
(SEQ ID NO: 110 and SEQ ID NO: 111), (SEQ ID NO: 110 and SEQ ID NO: 112), (SEQ ID NO: 111 and SEQ ID NO: 112)
(SEQ ID NO: 113 and SEQ ID NO: 114), (SEQ ID NO: 113 and SEQ ID NO: 115), (SEQ ID NO: 114 and SEQ ID NO: 115)
(SEQ ID NO: 116 and SEQ ID NO: 117), (SEQ ID NO: 116 and SEQ ID NO: 118), (SEQ ID NO: 117 and SEQ ID NO: 118).

As previously for embodiments A, B, C, D, E and F, and in one particular method, a minimum detection threshold of the toxic algae of the genus Karenia of less than 200 active living cells per litre of sample (cells/L) also corresponds to a minimum detection threshold of between 0.01 ng and 0.09 ng RNA per litre of sample.

In the same way, in a particular embodiment, the duration of the implementation of the said detection method is less than one hour.

For example, embodiment G allows the detection of:
G1: from Alexandrium and Karenia (combined with A)
G2: from Alexandrium, Dinophysis and Karenia (combined with B)
G3: from Alexandrium, Pseudo-nitzschia and Karenia (combined with C1)
G4: from Alexandrium, Dinophysis, Pseudo-nitzschia and Karenia (combined with C2)
G5: from Alexandrium, Prorocentrum and Karenia (combined with D1)
G6: from Alexandrium, Dinophysis, Prorocentrum and Karenia (combined with D2)
G7: from Alexandrium, Pseudo-nitzschia, Prorocentrum and Karenia (combined with D3)
G8: from Alexandrium, Dinophysis, Pseudo-nitzschia, Prorocentrum and Karenia (combined with D4)
G9: from Alexandrium, Chattonella and Karenia (combined with E1)
G10: from Alexandrium, Dinophysis, Chattonella and Karenia (combined with E2)
G11: from Alexandrium, Pseudo-nitzschia, Chattonella and Karenia (combined with E3)
G12: from Alexandrium, Dinophysis, Pseudo-nitzschia, Chattonella and Karenia (combined with E4)
G13: from Alexandrium, Prorocentrum, Chattonella and Karenia (combined with E5)
G14: from Alexandrium, Dinophysis, Prorocentrum, Chattonella and Karenia (combined with E6)
G15: from Alexandrium, Pseudo-nitzschia, Prorocentrum, Chattonella and Karenia (combined with E7)
G16: from Alexandrium, Dinophysis, Pseudo-nitzschia, Prorocentrum, Chattonella and Karenia (combined with E8)
G17: from Alexandrium, Gymnodinium and Karenia (combined with F1)
G18: from Alexandrium, Dinophysis, Gymnodinium and Karenia (combined with F2)
G19: from Alexandrium, Pseudo-nitzschia, Gymnodinium and Karenia (combined with F3)
G20: from Alexandrium, Dinophysis, Pseudo-nitzschia, Gymnodinium and Karenia (combined with F4)
G21: from Alexandrium, Prorocentrum, Gymnodinium and Karenia (combined with F5)
G22: from Alexandrium, Dinophysis, Prorocentrum, Gymnodinium and Karenia (combined with F6)
G23: from Alexandrium, Pseudo-nitzschia, Prorocentrum, Gymnodinium and Karenia (combined with F7)
G24: from Alexandrium, Dinophysis, Pseudo-nitzschia, Prorocentrum, Gymnodinium and Karenia (combined with F8)
G25: from Alexandrium, Chattonella, Gymnodinium and Karenia (combined with F9)
G26: from Alexandrium, Dinophysis, Chattonella, Gymnodinium and Karenia (combined with F10)
G27: from Alexandrium, Pseudo-nitzschia, Chattonella, Gymnodinium and Karenia (combined with F11)
G28: from Alexandrium, Dinophysis, Pseudo-nitzschia, Chattonella, Gymnodinium and Karenia (combined with F12)
G29: from Alexandrium, Prorocentrum, Chattonella, Gymnodinium and Karenia (combined with F13)
G30: from Alexandrium, Dinophysis, Prorocentrum, Chattonella, Gymnodinium and Karenia (combined with F14)
G31: from Alexandrium, Pseudo-nitzschia, Prorocentrum, Chattonella, Gymnodinium and Karenia (combined with F15)
G32: from Alexandrium, Dinophysis, Pseudo-nitzschia, Prorocentrum, Chattonella, Gymnodinium and Karenia (combined with F16)
   thanks to the use of specific probes of toxic algae of the genus Alexandrium, Dinophysis, Pseudo-nitzschia, Prorocentrum, Chattonella, Gymnodinium and Karenia.

### Embodiment H

In the same way, the invention also concerns one of the uses as described above according to embodiments A, B, C, D, E, F or G, comprising in addition the use of at least one pair of probes specific to toxic algae of the genus Lingulodinium for the implementation of a method for detecting active living cells of toxic algae in a sample likely to contain at least one toxic algae of the genus Alexandrium and/or Lingulodinium, the sequences of said probes being chosen from x elements of one of the following sets:
(SEQ ID NO: 119, SEQ ID NO: 120 or SEQ ID NO: 121)
(SEQ ID NO: 122, SEQ ID NO: 123 or SEQ ID NO: 124)
(SEQ ID NO: 125, SEQ ID NO: 126 or SEQ ID NO: 127)
   x being 3,
   or the sequences of said probes having at least 92% identity with the abovementioned sequences SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126 or SEQ ID NO: 127,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic algae of the genus Lingulodinium optionally present in said sample to form a complex,
   the minimum detection limit of the toxic algae of the genus Lingulodinium being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In a particular embodiment, the invention concerns the use of at least one pair of probes specific to toxic algae of the genus Lingulodinium as described above for the implementation of a method for the detection of active living cells of toxic algae in a sample likely to contain at least one toxic algae of the genus Lingulodinium in which the sequences of the probes of the said pairs are as follows:
(SEQ ID NO: 119 and SEQ ID NO: 120), (SEQ ID NO: 119 and SEQ ID NO: 121), (SEQ ID NO: 120 and SEQ ID NO: 121)
(SEQ ID NO: 122 and SEQ ID NO: 123), (SEQ ID NO: 122 and SEQ ID NO: 124), (SEQ ID NO: 123 and SEQ ID NO: 124)
(SEQ ID NO: 125 and SEQ ID NO: 126), (SEQ ID NO: 125 and SEQ ID NO: 127), (SEQ ID NO: 126 and SEQ ID NO: 127).

As previously for embodiments A, B, C, D, E, F and G, and in one particular embodiment, a minimum detection threshold of the toxic algae of the genus Lingulodinium of less than 200 active living cells per litre of sample (water, culture) (cells/L) also corresponds to a minimum detection threshold of between 0.01 ng and 0.09 ng RNA per litre of sample (water, culture).

In the same way, in a particular embodiment, the duration of the implementation of the said detection method is less than one hour.

Thus, the H embodiment allows the detection of:
H1: from Alexandrium and Lingulodinium (combined with A)
H2: from Alexandrium, Dinophysis and Lingulodinium (combined with B)
H3: of Alexandrium, Pseudo-nitzschia and Lingulodinium (combined with C1)
H4: of Alexandrium, Dinophysis, Pseudo-nitzschia and Lingulodinium (combined with C2)
H5: from Alexandrium, Prorocentrum and Lingulodinium (combined with D1)
H6: from Alexandrium, Dinophysis, Prorocentrum and Lingulodinium (combined with D2)
H7: from Alexandrium, Pseudo-nitzschia, Prorocentrum and Lingulodinium (combined with D3)
H8: from Alexandrium, Dinophysis, Pseudo-nitzschia, Prorocentrum and Lingulodinium (combined with D4)
H9: from Alexandrium, Chattonella and Lingulodinium (combined with E1)
H10: from Alexandrium, Dinophysis, Chattonella and Lingulodinium (combined with E2)
H11: from Alexandrium, Pseudo-nitzschia, Chattonella and Lingulodinium (combined with E3)
H12: of Alexandrium, Dinophysis, Pseudo-nitzschia, Chattonella and Lingulodinium (combined with E4)
H13: from Alexandrium, Prorocentrum, Chattonella and Lingulodinium (combined with E5)
H14: from Alexandrium, Dinophysis, Prorocentrum, Chattonella and Lingulodinium (combined with E6)
H15: from Alexandrium, Pseudo-nitzschia, Prorocentrum, Chattonella and Lingulodinium (combined with E7)
H16: from Alexandrium, Dinophysis, Pseudo-nitzschia, Prorocentrum, Chattonella and Lingulodinium (combined with E8)
H17: from Alexandrium, Gymnodinium and Lingulodinium (combined with F1)
H18: from Alexandrium, Dinophysis, Gymnodinium and Lingulodinium (combined with F2)
H19: from Alexandrium, Pseudo-nitzschia, Gymnodinium and Lingulodinium (combined with F3)
H20: of Alexandrium, Dinophysis, Pseudo-nitzschia, Gymnodinium and Lingulodinium (combined with F4)
H21: from Alexandrium, Prorocentrum, Gymnodinium and Lingulodinium (combined with F5)
H22: from Alexandrium, Dinophysis, Prorocentrum, Gymnodinium and Lingulodinium (combined with F6)
H23: from Alexandrium, Pseudo-nitzschia, Prorocentrum, Gymnodinium and Lingulodinium (combined with F7)
H24: of Alexandrium, Dinophysis, Pseudo-nitzschia, Prorocentrum, Gymnodinium and Lingulodinium (combined with F8)
H25: from Alexandrium, Chattonella, Gymnodinium and Lingulodinium (combined with F9)
H26: from Alexandrium, Dinophysis, Chattonella, Gymnodinium and Lingulodinium (combined with F10)
H27: from Alexandrium, Pseudo-nitzschia, Chattonella, Gymnodinium and Lingulodinium (combined with F11)
H28: of Alexandrium, Dinophysis, Pseudo-nitzschia, Chattonella, Gymnodinium and Lingulodinium (combined with F12)
H29: from Alexandrium, Prorocentrum, Chattonella, Gymnodinium and Lingulodinium (combined with F13)
H30: from Alexandrium, Dinophysis, Prorocentrum, Chattonella, Gymnodinium and Lingulodinium (combined with F14)
H31: from Alexandrium, Pseudo-nitzschia, Prorocentrum, Chattonella, Gymnodinium and Lingulodinium (combined with F15)
H32: from Alexandrium, Dinophysis, Pseudo-nitzschia, Prorocentrum, Chattonella, Gymnodinium and Lingulodinium (combined with F16)
H33: from Alexandrium, Karenia and Lingulodinium (combination with G1)
H34: from Alexandrium, Dinophysis, Karenia and Lingulodinium (combined with G2)
H35: from Alexandrium, Pseudo-nitzschia, Karenia and Lingulodinium (combined with G3)
H36: from Alexandrium, Dinophysis, Pseudo-nitzschia, Karenia and Lingulodinium (combined with G4)
H37: from Alexandrium, Prorocentrum, Karenia and Lingulodinium (combined with G5)
H38: from Alexandrium, Dinophysis, Prorocentrum, Karenia and Lingulodinium (combined with G6)
H39: from Alexandrium, Pseudo-nitzschia, Prorocentrum, Karenia and Lingulodinium (combined with G7)
H40: from Alexandrium, Dinophysis, Pseudo-nitzschia, Prorocentrum, Karenia and Lingulodinium (combined with G8)
H41: from Alexandrium, Chattonella, Karenia and Lingulodinium (combined with G9)
H42: from Alexandrium, Dinophysis, Chattonella, Karenia and Lingulodinium (combined with G10)
H43: from Alexandrium, Pseudo-nitzschia, Chattonella, Karenia and Lingulodinium (combined with G11)
H44: of Alexandrium, Dinophysis, Pseudo-nitzschia, Chattonella, Karenia and Lingulodinium (combined with G12)
H45: from Alexandrium, Prorocentrum, Chattonella, Karenia and Lingulodinium (combined with G13)
H46: from Alexandrium, Dinophysis, Prorocentrum, Chattonella, Karenia and Lingulodinium (combined with G14)
H47: from Alexandrium, Pseudo-nitzschia, Prorocentrum, Chattonella, Karenia and Lingulodinium (combined with G15)
H48: from Alexandrium, Dinophysis, Pseudo-nitzschia, Prorocentrum, Chattonella, Karenia and Lingulodinium (combined with G16)
H49: from Alexandrium, Gymnodinium, Karenia and Lingulodinium (combined with G17)
H50: from Alexandrium, Dinophysis, Gymnodinium, Karenia and Lingulodinium (combined with G18)
H51: from Alexandrium, Pseudo-nitzschia, Gymnodinium, Karenia and Lingulodinium (combined with G19)
H52: from Alexandrium, Dinophysis, Pseudo-nitzschia, Gymnodinium, Karenia and Lingulodinium (combined with G20)
H53: from Alexandrium, Prorocentrum, Gymnodinium, Karenia and Lingulodinium (combined with G21)
H54: from Alexandrium, Dinophysis, Prorocentrum, Gymnodinium, Karenia and Lingulodinium (combined with G22)
H55: from Alexandrium, Pseudo-nitzschia, Prorocentrum, Gymnodinium, Karenia and Lingulodinium (combined with G23)
H56: from Alexandrium, Dinophysis, Pseudo-nitzschia, Prorocentrum, Gymnodinium, Karenia and Lingulodinium (combined with G24)
H57: from Alexandrium, Chattonella, Gymnodinium, Karenia and Lingulodinium (combined with G25)
H58: from Alexandrium, Dinophysis, Chattonella, Gymnodinium, Karenia and Lingulodinium (combined with G26)
H59: from Alexandrium, Pseudo-nitzschia, Chattonella, Gymnodinium, Karenia and Lingulodinium (combined with G27)
H60: from Alexandrium, Dinophysis, Pseudo-nitzschia, Chattonella, Gymnodinium, Karenia and Lingulodinium (combined with G28)
H61: from Alexandrium, Prorocentrum, Chattonella, Gymnodinium, Karenia and Lingulodinium (combined with G29)
H62: from Alexandrium, Dinophysis, Prorocentrum, Chattonella, Gymnodinium, Karenia and Lingulodinium (combined with G30)
H63: from Alexandrium, Pseudo-nitzschia, Prorocentrum, Chattonella, Gymnodinium, Karenia and Lingulodinium (combined with G31)
H64: from Alexandrium, Dinophysis, Pseudo-nitzschia, Prorocentrum, Chattonella, Gymnodinium, Karenia and Lingulodinium (combined with G32)
   thanks to the use of specific probes of toxic algae of the genus Alexandrium, Dinophysis, Pseudo-nitzschia, Prorocentrum, Chattonella, Gymnodinium, Karenia and Lingulodinium.

### Embodiment I

In the same way, the invention also concerns one of the uses as described above according to embodiments A, B, C, D, E, F, G or H, comprising in addition the use of at least one pair of probes specific to toxic algae of the genus Heterosigma for the implementation of a method for the detection of active living cells of toxic algae in a sample likely to contain at least one toxic alga of the genus Alexandrium and/or Heterosigma, the sequences of said probes being chosen from x elements of one of the following sets:
(SEQ ID NO: 128 and SEQ ID NO: 129)
   x being 2,
   or the sequences of said probes having at least 92% identity with the aforementioned sequences SEQ ID NO: 128 or SEQ ID NO: 129,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic alga of the genus Heterosigma optionally present in said sample to form a complex,
   the minimum detection threshold of the toxic algae of the genus Heterosigma being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

As previously for embodiments A, B, C, D, E, F, G and H, and in one particular embodiment, a minimum detection threshold of the toxic algae of the genus Heterosigma of less than 200 active living cells per litre of sample (cells/L) also corresponds to a minimum detection threshold of between 0.01 ng and 0.09 ng RNA per litre of sample.

In the same way, in a particular embodiment, the duration of the implementation of the said detection method is less than one hour.

For example, embodiment I allows the detection:
I1: from Alexandrium and Heterosigma (combined with A)
I2: from Alexandrium, Dinophysis and Heterosigma (combined with B)
I3: of Alexandrium, Pseudo-nitzschia and Heterosigma (combined with C1)
I4: of Alexandrium, Dinophysis, Pseudo-nitzschia and Heterosigma (combined with C2)
I5: from Alexandrium, Prorocentrum and Heterosigma (combined with D1)
I6: from Alexandrium, Dinophysis, Prorocentrum and Heterosigma (combined with D2)
I7: from Alexandrium, Pseudo-nitzschia, Prorocentrum and Heterosigma (combined with D3)
I8: from Alexandrium, Dinophysis, Pseudo-nitzschia, Prorocentrum and Heterosigma (combined with D4)
I9: from Alexandrium, Chattonella and Heterosigma (combined with E1)
I10: from Alexandrium, Dinophysis, Chattonella and Heterosigma (combined with E2)
I11: from Alexandrium, Pseudo-nitzschia, Chattonella and Heterosigma (combined with E3)
I12: from Alexandrium, Dinophysis, Pseudo-nitzschia, Chattonella and Heterosigma (combined with E4)
I13: from Alexandrium, Prorocentrum, Chattonella and Heterosigma (combined with E5)
I14: from Alexandrium, Dinophysis, Prorocentrum, Chattonella and Heterosigma (combined with E6)
I15: from Alexandrium, Pseudo-nitzschia, Prorocentrum, Chattonella and Heterosigma (combined with E7)
I16: from Alexandrium, Dinophysis, Pseudo-nitzschia, Prorocentrum, Chattonella and Heterosigma (combined with E8)
I17: from Alexandrium, Gymnodinium and Heterosigma (combined with F1)
I18: from Alexandrium, Dinophysis, Gymnodinium and Heterosigma (combined with F2)
I19: from Alexandrium, Pseudo-nitzschia, Gymnodinium and Heterosigma (combined with F3)
I20: of Alexandrium, Dinophysis, Pseudo-nitzschia, Gymnodinium and Heterosigma (combined with F4)
I21: from Alexandrium, Prorocentrum, Gymnodinium and Heterosigma (combined with F5)
I22: from Alexandrium, Dinophysis, Prorocentrum, Gymnodinium and Heterosigma (combined with F6)
I23: from Alexandrium, Pseudo-nitzschia, Prorocentrum, Gymnodinium and Heterosigma (combined with F7)
I24: from Alexandrium, Dinophysis, Pseudo-nitzschia, Prorocentrum, Gymnodinium and Heterosigma (combined with F8)
I25: from Alexandrium, Chattonella, Gymnodinium and Heterosigma (combined with F9)
I26: from Alexandrium, Dinophysis, Chattonella, Gymnodinium and Heterosigma (combined with F10)
I27: from Alexandrium, Pseudo-nitzschia, Chattonella, Gymnodinium and Heterosigma (combined with F11)
I28: of Alexandrium, Dinophysis, Pseudo-nitzschia, Chattonella, Gymnodinium and Heterosigma (combined with F12)
I29: from Alexandrium, Prorocentrum, Chattonella, Gymnodinium and Heterosigma (combined with F13)
I30: from Alexandrium, Dinophysis, Prorocentrum, Chattonella, Gymnodinium and Heterosigma (combined with F14)
I31: from Alexandrium, Pseudo-nitzschia, Prorocentrum, Chattonella, Gymnodinium and Heterosigma (combined with F15)
I32: from Alexandrium, Dinophysis, Pseudo-nitzschia, Prorocentrum, Chattonella, Gymnodinium and Heterosigma (combined with F16)
I33: from Alexandrium, Karenia and Heterosigma (combined with G1)
I34: from Alexandrium, Dinophysis, Karenia and Heterosigma (combined with G2)
I35: from Alexandrium, Pseudo-nitzschia, Karenia and Heterosigma (combined with G3)
I36: from Alexandrium, Dinophysis, Pseudo-nitzschia, Karenia and Heterosigma (combined with G4)
I37: from Alexandrium, Prorocentrum, Karenia and Heterosigma (combined with G5)
I38: from Alexandrium, Dinophysis, Prorocentrum, Karenia and Heterosigma (combined with G6)
I39: from Alexandrium, Pseudo-nitzschia, Prorocentrum, Karenia and Heterosigma (combined with G7)
I40: from Alexandrium, Dinophysis, Pseudo-nitzschia, Prorocentrum, Karenia and Heterosigma (combined with G8)
I41: from Alexandrium, Chattonella, Karenia and Heterosigma (combined with G9)
I42: from Alexandrium, Dinophysis, Chattonella, Karenia and Heterosigma (combined with G10)
I43: from Alexandrium, Pseudo-nitzschia, Chattonella, Karenia and Heterosigma (combined with G11)
I44: from Alexandrium, Dinophysis, Pseudo-nitzschia, Chattonella, Karenia and Heterosigma (combined with G12)
I45: from Alexandrium, Prorocentrum, Chattonella, Karenia and Heterosigma (combined with G13)
I46: from Alexandrium, Dinophysis, Prorocentrum, Chattonella, Karenia and Heterosigma (combined with G14)
I47: from Alexandrium, Pseudo-nitzschia, Prorocentrum, Chattonella, Karenia and Heterosigma (combined with G15)
I48: from Alexandrium, Dinophysis, Pseudo-nitzschia, Prorocentrum, Chattonella, Karenia and Heterosigma (combined with G16)
I49: from Alexandrium, Gymnodinium, Karenia and Heterosigma (combined with G17)
I50: from Alexandrium, Dinophysis, Gymnodinium, Karenia and Heterosigma (combined with G18)
I51: from Alexandrium, Pseudo-nitzschia, Gymnodinium, Karenia and Heterosigma (combined with G19)
I52: from Alexandrium, Dinophysis, Pseudo-nitzschia, Gymnodinium, Karenia and Heterosigma (combined with G20)
I53: from Alexandrium, Prorocentrum, Gymnodinium, Karenia and Heterosigma (combined with G21)
I54: from Alexandrium, Dinophysis, Prorocentrum, Gymnodinium, Karenia and Heterosigma (combined with G22)
I55: from Alexandrium, Pseudo-nitzschia, Prorocentrum, Gymnodinium, Karenia and Heterosigma (combined with G23)
I56: from Alexandrium, Dinophysis, Pseudo-nitzschia, Prorocentrum, Gymnodinium, Karenia and Heterosigma (combined with G24)
I57: from Alexandrium, Chattonella, Gymnodinium, Karenia and Heterosigma (combined with G25)
I58: from Alexandrium, Dinophysis, Chattonella, Gymnodinium, Karenia and Heterosigma (combined with G26)
I59: from Alexandrium, Pseudo-nitzschia, Chattonella, Gymnodinium, Karenia and Heterosigma (combined with G27)
I60: from Alexandrium, Dinophysis, Pseudo-nitzschia, Chattonella, Gymnodinium, Karenia and Heterosigma (combined with G28)
I61: from Alexandrium, Prorocentrum, Chattonella, Gymnodinium, Karenia and Heterosigma (combined with G29)
I62: from Alexandrium, Dinophysis, Prorocentrum, Chattonella, Gymnodinium, Karenia and Heterosigma (combined with G30)
I63: from Alexandrium, Pseudo-nitzschia, Prorocentrum, Chattonella, Gymnodinium, Karenia and Heterosigma (combined with G31)
I64: from Alexandrium, Dinophysis, Pseudo-nitzschia, Prorocentrum, Chattonella, Gymnodinium, Karenia and Heterosigma (combined with G32)
I65: from Alexandrium, Lingulodinium and Heterosigma (combined with H1)
I66: of Alexandrium, Dinophysis, Lingulodinium and Heterosigma (combined with H2)
I67: of Alexandrium, Pseudo-nitzschia, Lingulodinium and Heterosigma (combined with H3)
I68: of Alexandrium, Dinophysis, Pseudo-nitzschia, Lingulodinium and Heterosigma (combined with H4)
I69: from Alexandrium, Prorocentrum, Lingulodinium and Heterosigma (combined with H5)
I70: from Alexandrium, Dinophysis, Prorocentrum, Lingulodinium and Heterosigma (combined with H6)
I71: from Alexandrium, Pseudo-nitzschia, Prorocentrum, Lingulodinium and Heterosigma (combined with H7)
I72: of Alexandrium, Dinophysis, Pseudo-nitzschia, Prorocentrum, Lingulodinium and Heterosigma (combined with H8)
I73: from Alexandrium, Chattonella and Lingulodinium (combined with H9)
I74: from Alexandrium, Dinophysis, Chattonella, Lingulodinium and Heterosigma (combined with H10)
I75: of Alexandrium, Pseudo-nitzschia, Chattonella, Lingulodinium and Heterosigma (combined with H11)
I76: of Alexandrium, Dinophysis, Pseudo-nitzschia, Chattonella, Lingulodinium and Heterosigma (combined with H12)
I76: from Alexandrium, Prorocentrum, Chattonella, Lingulodinium and Heterosigma (combined with H13)
I78: from Alexandrium, Dinophysis, Prorocentrum, Chattonella, Lingulodinium and Heterosigma (combined with H14)
I79: of Alexandrium, Pseudo-nitzschia, Prorocentrum, Chattonella, Lingulodinium and Heterosigma (combined with H15)
I80: of Alexandrium, Dinophysis, Pseudo-nitzschia, Prorocentrum, Chattonella, Lingulodinium and Heterosigma (combined with H16)
I81: from Alexandrium, Gymnodinium, Lingulodinium, and Heterosigma (combined with H17)
I82: of Alexandrium, Dinophysis, Gymnodinium, Lingulodinium and Heterosigma (combined with H18)
I83: of Alexandrium, Pseudo-nitzschia, Gymnodinium, Lingulodinium and Heterosigma (combined with H19)
I84: of Alexandrium, Dinophysis, Pseudo-nitzschia, Gymnodinium, Lingulodinium and Heterosigma (combined with H20)
I85: from Alexandrium, Prorocentrum, Gymnodinium, Lingulodinium and Heterosigma (combined with H21)
I86: from Alexandrium, Dinophysis, Prorocentrum, Gymnodinium, Lingulodinium and Heterosigma (combined with H22)
I87: from Alexandrium, Pseudo-nitzschia, Prorocentrum, Gymnodinium, Lingulodinium and Heterosigma (combined with H23)
I88: of Alexandrium, Dinophysis, Pseudo-nitzschia, Prorocentrum, Gymnodinium, Lingulodinium and Heterosigma (combined with H24)
I89: from Alexandrium, Chattonella, Gymnodinium, Lingulodinium and Heterosigma (combined with H25)
I90: from Alexandrium, Dinophysis, Chattonella, Gymnodinium, Lingulodinium and Heterosigma (combined with H26)
I91: of Alexandrium, Pseudo-nitzschia, Chattonella, Gymnodinium, Lingulodinium and Heterosigma (combined with H27)
I92: of Alexandrium, Dinophysis, Pseudo-nitzschia, Chattonella, Gymnodinium, Lingulodinium and Heterosigma (combined with H28)
I93: from Alexandrium, Prorocentrum, Chattonella, Gymnodinium, Lingulodinium and Heterosigma (combined with H29)
I94: from Alexandrium, Dinophysis, Prorocentrum, Chattonella, Gymnodinium, Lingulodinium and Heterosigma (combined with H30)
I95: from Alexandrium, Pseudo-nitzschia, Prorocentrum, Chattonella, Gymnodinium, Lingulodinium and Heterosigma (combined with H31)
I96: of Alexandrium, Dinophysis, Pseudo-nitzschia, Prorocentrum, Chattonella, Gymnodinium, Lingulodinium and Heterosigma (combined with H32)
I97: from Alexandrium, Karenia, Lingulodinium and Heterosigma (combined with H33)
I98: from Alexandrium, Dinophysis, Karenia, Lingulodinium and Heterosigma (combined with H34)
I99: from Alexandrium, Pseudo-nitzschia, Karenia, Lingulodinium and Heterosigma (combined with H35)
I100: of Alexandrium, Dinophysis, Pseudo-nitzschia, Karenia, Lingulodinium and Heterosigma (combined with H36)
I101: from Alexandrium, Prorocentrum, Karenia, Lingulodinium and Heterosigma (combined with H37)
I102: from Alexandrium, Dinophysis, Prorocentrum, Karenia, Lingulodinium and Heterosigma (combined with H38)
I103: from Alexandrium, Pseudo-nitzschia, Prorocentrum, Karenia, Lingulodinium and Heterosigma (combined with H39)
I104: of Alexandrium, Dinophysis, Pseudo-nitzschia, Prorocentrum, Karenia, Lingulodinium and Heterosigma (combined with H40)
I105: from Alexandrium, Chattonella, Karenia, Lingulodinium and Heterosigma (combined with H41)
I106: from Alexandrium, Dinophysis, Chattonella, Karenia, Lingulodinium and Heterosigma (combined with H42)
I107: of Alexandrium, Pseudo-nitzschia, Chattonella, Karenia, Lingulodinium and Heterosigma (combined with H43)
I108: of Alexandrium, Dinophysis, Pseudo-nitzschia, Chattonella, Karenia, Lingulodinium and Heterosigma (combined with H44)
I109: from Alexandrium, Prorocentrum, Chattonella, Karenia, Lingulodinium and Heterosigma (combined with H45)
I110: from Alexandrium, Dinophysis, Prorocentrum, Chattonella, Karenia, Lingulodinium and Heterosigma (combined with H46)
I111: from Alexandrium, Pseudo-nitzschia, Prorocentrum, Chattonella, Karenia, Lingulodinium and Heterosigma (combined with H47)
I112: of Alexandrium, Dinophysis, Pseudo-nitzschia, Prorocentrum, Chattonella, Karenia, Lingulodinium and Heterosigma (combined with H48)
I113: from Alexandrium, Gymnodinium, Karenia, Lingulodinium and Heterosigma (combined with H49)
I114: from Alexandrium, Dinophysis, Gymnodinium, Karenia, Lingulodinium and Heterosigma (combined with H50)
I115: of Alexandrium, Pseudo-nitzschia, Gymnodinium, Karenia, Lingulodinium and Heterosigma (combined with H51)
I116: of Alexandrium, Dinophysis, Pseudo-nitzschia, Gymnodinium, Karenia, Lingulodinium and Heterosigma (combined with H52)
I117: from Alexandrium, Prorocentrum, Gymnodinium, Karenia, Lingulodinium and Heterosigma (combined with H53)
I118: from Alexandrium, Dinophysis, Prorocentrum, Gymnodinium, Karenia, Lingulodinium and Heterosigma (combined with H54)
I119: from Alexandrium, Pseudo-nitzschia, Prorocentrum, Gymnodinium, Karenia, Lingulodinium and Heterosigma (combined with H55)
I120: of Alexandrium, Dinophysis, Pseudo-nitzschia, Prorocentrum, Gymnodinium, Karenia, Lingulodinium and Heterosigma (combined with H56)
I121: from Alexandrium, Chattonella, Gymnodinium, Karenia, Lingulodinium and Heterosigma (combined with H57)
I122: from Alexandrium, Dinophysis, Chattonella, Gymnodinium, Karenia, Lingulodinium and Heterosigma (combined with H58)
I123: of Alexandrium, Pseudo-nitzschia, Chattonella, Gymnodinium, Karenia, Lingulodinium and Heterosigma (combined with H59)
I124: of Alexandrium, Dinophysis, Pseudo-nitzschia, Chattonella, Gymnodinium, Karenia, Lingulodinium and Heterosigma (combined with H60)
I125: from Alexandrium, Prorocentrum, Chattonella, Gymnodinium, Karenia, Lingulodinium and Heterosigma (combined with H61)
I126: from Alexandrium, Dinophysis, Prorocentrum, Chattonella, Gymnodinium, Karenia Lingulodinium and Heterosigma (combined with H62)
I127: from Alexandrium, Pseudo-nitzschia, Prorocentrum, Chattonella, Gymnodinium, Karenia, Lingulodinium and Heterosigma (combined with H63)
I128: from Alexandrium, Dinophysis, Pseudo-nitzschia, Prorocentrum, Chattonella, Gymnodinium, Karenia, Lingulodinium and Heterosigma (combined with H64)
   thanks to the use of specific probes of toxic algae of the genus Alexandrium, Dinophysis, Pseudo-nitzschia, Prorocentrum, Chattonella, Gymnodinium, Karenia, Lingulodinium and Heterosigma.

“Percentage identity” in relation to a given sequence means the percentage of amino acids that are identical to those in a reference sequence and that are found in the same positions.

“At least 92% identity” means the ranges of at least 92%, at least 93%, at least 94%, at least 95%, at least 96%, at least 97%, at least 98%, at least 99% and 100% identity.

In all embodiments of this first aspect, and according to a particular embodiment, said capture probe is linked to at least one attachment molecule positioned 5′ from its sequence and said signal probe is linked to at least one marking molecule positioned 5′ from its sequence.

In all embodiments of this first aspect, and according to a particular embodiment, said capture probe is linked to at least one attachment molecule positioned 5′ from its sequence and said signal probe is linked to at least one marking molecule positioned 3′ from its sequence.

In all embodiments of this first aspect, and according to a particular embodiment, said capture probe is linked to at least one attachment molecule positioned 3′ from its sequence and said signal probe is linked to at least one marking molecule positioned 5′ from its sequence.

In all embodiments of this first aspect, and according to a particular embodiment, said capture probe is linked to at least one attachment molecule positioned in 3′ of its sequence and said signal probe is linked to at least one marking molecule positioned in 3′ of its sequence.

In all embodiments of this first aspect, the “at least one attachment molecule” can be chosen from a biotin, avidin, streptavidin molecule, a thiol group, an amine group and a carbon.

In all embodiments of to achieve this first aspect, and in one particularly preferred embodiment, the “at least one attachment molecule” is a biotin molecule.

In all embodiments of this first aspect, the “at least one marking molecule” may be chosen from a fluorochrome, a biotin, a biotin-bound molecule, digoxigenin, an enzyme using a chemiluminescent substrate, an enzyme using a chromogenic substrate or an enzyme using an electrochemical oxidation substrate.

In all embodiments of this first aspect, and in one particularly preferred embodiment, the “at least one marking molecule” is digoxigenin.

Thus, in a particular embodiment, the invention relates to the use as described above in which,

said capture probe is linked to at least one attachment molecule positioned 5′ of its sequence and said signal probe is linked to at least one marker molecule positioned 5′ of its sequence, or
said capture probe is linked to at least one attachment molecule positioned 5′ of its sequence and said signal probe is linked to at least one marker molecule positioned 3′ of its sequence, or
said capture probe is linked to at least one attachment molecule positioned 3′ of its sequence and said signal probe is linked to at least one marker molecule positioned 5′ of its sequence, or
said capture probe is linked to at least one attachment molecule positioned 3′ of its sequence and said signal probe is linked to at least one labelling molecule positioned 3′ of its sequence,
said “at least one attachment molecule” being in particular selected from a biotin, avidin, streptavidin molecule, a thiol group, an amine group and a carbon, preferably a biotin molecule,
wherein said “at least one marking molecule” being in particular selected from a fluorochrome, a biotin, a biotin-bound molecule, digoxigenin, an enzyme using a chemiluminescent substrate or an enzyme using a chromogenic substrate or an enzyme using an electrochemically oxidised substrate, preferably digoxigenin.

In all embodiments of this first aspect, the said fluorochrome can be chosen from the group consisting of: Alexa fluor, in particular Alexa fluor 350, 405, 430, 488, 500, 514, 532, 546, 555, 568, 594, 610, 633, 647, 660, 680, 700, 750 or 790, Fluorescein Isothiocyanate (FITC), Rhodamine, Allophycocyanine (APC) and Phycoerythrin (PE).

In all embodiments of this first aspect, said enzyme using a chemiluminescent substrate may be horseradish peroxidase (HRP) and said chemiluminescent substrate may be luminol, or said enzyme using a chemiluminescent substrate may be luciferase and said chemiluminescent substrate may be luciferin. In this case, the reaction of the enzyme and its substrate generates light which can be measured or read by a luminescence reader.

In all embodiments of this first aspect, the said enzyme using a chromogenic substrate can be alkaline phosphatase and the said chromogenic substrate can be Tetrazolium Nitroblue (NBT) or Bromochlorylindolophosphate (BCIP), said enzyme using a chromogenic substrate may be horseradish peroxidase (HRP) and said chromogenic substrate may be selected from 3,3′-Diaminobenzidine (DAB), 3,3′,5,5′-Tetramethylbenzidine (TMB), or 2,2′-azino-bis(3-ethylbenzothiazoline-6-sulphonic acid) (ABTS). In this case, the enzyme oxidizes a substrate, which, when reduced, produces a coloured precipitate, which can be measured by an absorbance meter.

In all embodiments of this first aspect, said enzyme using an electrochemically oxidized substrate can be horseradish peroxidase (HRP) and said electrochemically oxidized substrate can be 3,3′,5,5′-Tetramethylbenzidine (TMB). In this case, the enzyme (e.g. Horseradish Peroxidase), reacts in the presence of H₂O₂, and oxidises a substrate (e.g. TMB) which, when reduced, produces an electrical potential difference. This electrical potential difference can be measured by an electrode.

In all embodiments of this first aspect, the sample may be a sample of seawater, brackish water, culture media or microalgae culture produced for commercial purposes.

The term “seawater sample” refers to a volume of water from the sea and oceans that contains living organisms such as phytoplankton and zooplankton.

The term “brackish water sample” refers to a volume of water resulting from the meeting of fresh and salt water bodies, such as a river estuary, a lagoon, a basin.

The term “culture medium” refers to a support that allows the culture of microorganisms such as microalgae, bacteria, yeasts.

The term “microalgae culture” refers to the management of an aquatic ecosystem in order to promote the production of one or more species of commercial interest such as unicellular or colonial microscopic algae.

In a second aspect, the invention concerns pairs of probes for the detection of active living cells of toxic algae.

Thus, in this second aspect, the invention concerns at least one pair of probes for the detection of active living cells of toxic algae of the genus Alexandrium, the sequences of which are chosen from x elements of one of the following sets:
(SEQ ID NO: 1, SEQ ID NO: 2 or SEQ ID NO: 3)
(SEQ ID NO: 4 and SEQ ID NO: 5)
(SEQ ID NO: 6 and SEQ ID NO: 7)
(SEQ ID NO: 8, SEQ ID NO: 9 or SEQ ID NO: 10)
(SEQ ID NO: 11, SEQ ID NO: 12 or SEQ ID NO: 13)
(SEQ ID NO: 14, SEQ ID NO: 15 or SEQ ID NO: 16)
(SEQ ID NO: 17, SEQ ID NO: 18 or SEQ ID NO: 19)
(SEQ ID NO: 20, SEQ ID NO: 21 or SEQ ID NO: 22),
(SEQ ID NO: 23, SEQ ID NO: 24 or SEQ ID NO: 25),
(SEQ ID NO: 26, SEQ ID NO: 27 or SEQ ID NO: 28)
   x being 2 or 3,
   or whose sequences have at least 92% identity with the above-mentioned sequences SEQ ID NO: 1 to SEQ ID NO: 28.

In a particular embodiment, the invention concerns at least one pair of probes for the detection of active living cells of toxic algae of the genus Alexandrium, the sequences of the probes of the said pairs are as follows:
(SEQ ID NO: 1 and SEQ ID NO: 2), (SEQ ID NO: 1 and SEQ ID NO: 3), (SEQ ID NO: 2 and SEQ ID NO: 3)
(SEQ ID NO: 4 and SEQ ID NO: 5)
(SEQ ID NO: 6 and SEQ ID NO: 7)
(SEQ ID NO: 8 and SEQ ID NO: 9), (SEQ ID NO: 8 and SEQ ID NO: 10), (SEQ ID NO: 9 and SEQ ID NO: 10)
(SEQ ID NO: 11 and SEQ ID NO: 12), (SEQ ID NO: 11 and SEQ ID NO: 13), (SEQ ID NO: 12 and SEQ ID NO: 13)
(SEQ ID NO: 14 and SEQ ID NO: 15), (SEQ ID NO: 14 and SEQ ID NO: 16), (SEQ ID NO: 15 and SEQ ID NO: 16)
(SEQ ID NO: 17 and SEQ ID NO: 18), (SEQ ID NO: 17 and 19), (SEQ ID NO: 18 and SEQ ID NO: 19)
(SEQ ID NO: 20 and SEQ ID NO: 21), (SEQ ID NO: 20 and SEQ ID NO: 22), (SEQ ID NO: 21 and SEQ ID NO: 22), or
(SEQ ID NO: 23 and SEQ ID NO: 24), (SEQ ID NO: 23 and SEQ ID NO: 25), (SEQ ID NO: 24 and SEQ ID NO: 25)
(SEQ ID NO: 26 and SEQ ID NO: 27), (SEQ ID NO: 26 and SEQ ID NO: 28), (SEQ ID NO: 27 and SEQ ID NO: 28).

In this second aspect, the invention also concerns at least one pair of probes for the detection of active living cells of toxic algae of the genus Dinophysis, the sequences of which are selected from x elements of one of the following sets:
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44 or SEQ ID NO: 45), or
(SEQ ID NO: 46, SEQ ID NO: 47 or SEQ ID NO: 48)
   x being 2 or 3,
   or whose sequences have at least 92% identity with the above-mentioned sequences SEQ ID NO: 29 to SEQ ID NO: 48.

In a particular embodiment, the invention concerns at least one pair of probes for the detection of active living cells of toxic algae of the genus Dinophysis, the sequences of the probes of the said pairs are as follows:
(SEQ ID NO: 29 and SEQ ID NO: 30), (SEQ ID NO: 29 and SEQ ID NO: 31), (SEQ ID NO: 30 and SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40 and SEQ ID NO: 41), (SEQ ID NO: 40 and SEQ ID NO: 42), (SEQ ID NO: 41 and SEQ ID NO: 42)
(SEQ ID NO: 43 and SEQ ID NO: 44), (SEQ ID NO: 43 and SEQ ID NO: 45), (SEQ ID NO: 44 and SEQ ID NO: 45)
(SEQ ID NO: 46 and SEQ ID NO: 47), (SEQ ID NO: 46 and SEQ ID NO: 48), (SEQ ID NO: 47 and SEQ ID NO: 48).

In this second aspect, the invention also concerns at least one pair of probes for the detection of active living cells of toxic algae of the genus Pseudo-nitzschia, the sequences of which are selected from x elements of one of the following sets:
(SEQ ID NO: 49 and SEQ ID NO: 50)
(SEQ ID NO: 51 and SEQ ID NO: 52)
(SEQ ID NO: 53, SEQ ID NO: 54 or SEQ ID NO: 55)
(SEQ ID NO: 56, SEQ ID NO: 57 or SEQ ID NO: 58)
(SEQ ID NO: 59, SEQ ID NO: 60 or SEQ ID NO: 61)
   x being 2 or 3,
   or whose sequences have at least 92% identity with the above-mentioned sequences SEQ ID NO: 49 to SEQ ID NO: 61.

In a particular embodiment, the invention concerns at least one pair of probes for the detection of Pseudo-nitzschia, the sequences of the probes of said pairs are as follows:
(SEQ ID NO: 49 and SEQ ID NO: 50)
(SEQ ID NO: 51 and SEQ ID NO: 52)
(SEQ ID NO: 53 and SEQ ID NO: 54), (SEQ ID NO: 53 and SEQ ID NO: 55), (SEQ ID NO: 54 and SEQ ID NO: 55)
(SEQ ID NO: 56 and SEQ ID NO: 57), (SEQ ID NO: 56 and SEQ ID NO: 58), (SEQ ID NO: 57 and SEQ ID NO: 58)
(SEQ ID NO: 59 and SEQ ID NO: 60), (SEQ ID NO: 59 and SEQ ID NO: 61), (SEQ ID NO: 60 and SEQ ID NO: 61).

In this second aspect, the invention also concerns at least one pair of probes for the detection of active living cells of toxic algae of the genus Prorocentrum, the sequences of which are selected from x elements of one of the following sets:
(SEQ ID NO: 62, SEQ ID NO: 63 or SEQ ID NO: 64)
(SEQ ID NO: 65, SEQ ID NO: 66 or SEQ ID NO: 67)
(SEQ ID NO: 68, SEQ ID NO: 69 or SEQ ID NO: 70)
(SEQ ID NO: 71, SEQ ID NO: 72 or SEQ ID NO: 73)
   x being 3,
   or whose sequences have at least 92% identity with the above-mentioned sequences SEQ ID NO: 62 to SEQ ID NO: 73.

In a particular embodiment, the invention concerns at least one pair of probes for the detection of active living cells of toxic algae of the genus Prorocentrum, the sequences of the probes of the said pairs are as follows:
(SEQ ID NO: 62 and SEQ ID NO: 63), (SEQ ID NO: 62 and SEQ ID NO: 64), (SEQ ID NO: 63 and SEQ ID NO: 64)
(SEQ ID NO: 65 and SEQ ID NO: 66), (SEQ ID NO: 65 and SEQ ID NO: 67), (SEQ ID NO: 66 and SEQ ID NO: 67)
(SEQ ID NO: 68 and SEQ ID NO: 69), (SEQ ID NO: 68 and SEQ ID NO: 70), (SEQ ID NO: 69 and SEQ ID NO: 70)
(SEQ ID NO: 71 and SEQ ID NO: 72), (SEQ ID NO: 71 and SEQ ID NO: 73), (SEQ ID NO: 72 and SEQ ID NO: 73).

In this second aspect, the invention also concerns at least one pair of probes for the detection of active living cells of toxic algae of the genus Chattonella, the sequences of which are selected from x elements of one of the following sets:
(SEQ ID NO: 74, SEQ ID NO: 75 or SEQ ID NO: 76)
(SEQ ID NO: 77, SEQ ID NO: 78 or SEQ ID NO: 79)
(SEQ ID NO: 80, SEQ ID NO: 81 or SEQ ID NO: 82)
   x being 3,
   or whose sequences have at least 92% identity with the above-mentioned sequences SEQ ID NO: 74 to SEQ ID NO: 82.

In a particular embodiment, the invention concerns at least one pair of probes for the detection of active living cells of toxic algae of the genus Chattonella, the sequences of the probes of the said pairs are as follows:
(SEQ ID NO: 74 and SEQ ID NO: 75), (SEQ ID NO: 74 and SEQ ID NO: 76), (SEQ ID NO: 75 and SEQ ID NO: 76)
(SEQ ID NO: 77 and SEQ ID NO: 78), (SEQ ID NO: 77 and SEQ ID NO: 79), (SEQ ID NO: 78 and SEQ ID NO: 79)
(SEQ ID NO: 80 and SEQ ID NO: 81), (SEQ ID NO: 80 and SEQ ID NO: 82), (SEQ ID NO: 81 and SEQ ID NO: 82).

In this second aspect, the invention also concerns at least one pair of probes for the detection of active living cells of toxic algae of the genus Gymnodinium, the sequences of which are selected from x elements of one of the following sets:
(SEQ ID NO: 83, SEQ ID NO: 84 or SEQ ID NO: 85)
(SEQ ID NO: 86, SEQ ID NO: 87 or SEQ ID NO: 88)
(SEQ ID NO: 89, SEQ ID NO: 90 or SEQ ID NO: 91)
(SEQ ID NO: 92, SEQ ID NO: 93 or SEQ ID NO: 94)
   x being 3,
   or whose sequences have at least 92% identity with the above-mentioned sequences SEQ ID NO: 83 to SEQ ID NO: 94.

In a particular embodiment, the invention concerns at least one pair of probes for the detection of active living cells of toxic algae of the genus Gymnodinium, the sequences of the probes of the said pairs are as follows:
(SEQ ID NO: 83 and SEQ ID NO: 84), (SEQ ID NO: 83 and SEQ ID NO: 85), (SEQ ID NO: 84 and SEQ ID NO: 85)
(SEQ ID NO: 86 and SEQ ID NO: 87), (SEQ ID NO: 86 and SEQ ID NO: 88), (SEQ ID NO: 87 and SEQ ID NO: 88)
(SEQ ID NO: 89 and SEQ ID NO: 90), (SEQ ID NO: 89 and SEQ ID NO: 91), (SEQ ID NO: 90 and SEQ ID NO: 91)
(SEQ ID NO: 92 and SEQ ID NO: 93), (SEQ ID NO: 92 and SEQ ID NO: 94), (SEQ ID NO: 93 and SEQ ID NO: 94).

In this second aspect, the invention also concerns at least one pair of probes for the detection of active living cells of toxic algae of the genus Karenia, the sequences of which are selected from x elements of one of the following sets:
(SEQ ID NO: 95, SEQ ID NO: 96 or SEQ ID NO: 97)
(SEQ ID NO: 98, SEQ ID NO: 99 or SEQ ID NO: 100)
(SEQ ID NO: 101, SEQ ID NO: 102 or SEQ ID NO: 103)
(SEQ ID NO: 104, SEQ ID NO: 105 or SEQ ID NO: 106)
(SEQ ID NO: 107, SEQ ID NO: 108 or SEQ ID NO: 109)
(SEQ ID NO: 110, SEQ ID NO: 111 or SEQ ID NO: 112)
(SEQ ID NO: 113, SEQ ID NO: 114 or SEQ ID NO: 115)
(SEQ ID NO: 116, SEQ ID NO: 117 or SEQ ID NO: 118)
   x being 3,
   or whose sequences have at least 92% identity with the above-mentioned sequences SEQ ID NO: 95 to SEQ ID NO: 118.

In a particular embodiment, the invention concerns at least one pair of probes for the detection of active living cells of toxic algae of the genus Karenia, the sequences of the probes of the said pairs are as follows:
(SEQ ID NO: 95 and SEQ ID NO: 96), (SEQ ID NO: 95 and SEQ ID NO: 97), (SEQ ID NO: 96 and SEQ ID NO: 97)
(SEQ ID NO: 98 and SEQ ID NO: 99), (SEQ ID NO: 98 and SEQ ID NO: 100), (SEQ ID NO: 99 and SEQ ID NO: 100)
(SEQ ID NO: 101 and SEQ ID NO: 102), (SEQ ID NO: 101 and SEQ ID NO: 103), (SEQ ID NO: 102 and SEQ ID NO: 103)
(SEQ ID NO: 104 and SEQ ID NO: 105), (SEQ ID NO: 104 and SEQ ID NO: 106), (SEQ ID NO: 105 and SEQ ID NO: 106)
(SEQ ID NO: 107 and SEQ ID NO: 108), (SEQ ID NO: 107 and SEQ ID NO: 109), (SEQ ID NO: 108 and SEQ ID NO: 109)
(SEQ ID NO: 110 and SEQ ID NO: 111), (SEQ ID NO: 110 and SEQ ID NO: 112), (SEQ ID NO: 111 and SEQ ID NO: 112)
(SEQ ID NO: 113 and SEQ ID NO: 114), (SEQ ID NO: 113 and SEQ ID NO: 115), (SEQ ID NO: 114 and SEQ ID NO: 115)
(SEQ ID NO: 116 and SEQ ID NO: 117), (SEQ ID NO: 116 and SEQ ID NO: 118), (SEQ ID NO: 117 and SEQ ID NO: 118).

In this second aspect, the invention also concerns at least one pair of probes for the detection of active living cells of toxic algae of the genus Lingulodinium, the sequences of which are selected from x elements of one of the following sets:
(SEQ ID NO: 119, SEQ ID NO: 120 or SEQ ID NO: 121)
(SEQ ID NO: 122, SEQ ID NO: 123 or SEQ ID NO: 124)
(SEQ ID NO: 125, SEQ ID NO: 126 or SEQ ID NO: 127)
   x being 3,
   or whose sequences have at least 92% identity with the above-mentioned sequences

SEQ ID NO: 119 to SEQ ID NO: 127.

In a particular embodiment, the invention concerns at least one pair of probes for the detection of active living cells of toxic algae of the genus Lingulodinium, the sequences of the probes of the said pairs are as follows:
(SEQ ID NO: 119 and SEQ ID NO: 120), (SEQ ID NO: 119 and SEQ ID NO: 121), (SEQ ID NO: 120 and SEQ ID NO: 121)
(SEQ ID NO: 122 and SEQ ID NO: 123), (SEQ ID NO: 122 and SEQ ID NO: 124), (SEQ ID NO: 123 and SEQ ID NO: 124)
(SEQ ID NO: 125 and SEQ ID NO: 126), (SEQ ID NO: 125 and SEQ ID NO: 127), (SEQ ID NO: 126 and SEQ ID NO: 127).

In this second aspect, the invention also concerns at least one pair of probes for the detection of active living cells of toxic algae of the genus Heterosigma, the sequences of which are selected from x elements of one of the following sets:
(SEQ ID NO: 128 and SEQ ID NO: 129)
   x being 2,
   or whose sequences have at least 92% identity with the above-mentioned sequences SEQ ID NO: 128 to SEQ ID NO: 129.

According to all embodiments of this second aspect of the invention, one probe of said pair is a capture probe linked to at least one attachment molecule positioned at 3′ or 5′ of its sequence and the other probe of said pair is a signal probe linked to at least one marking molecule positioned at 3′ or 5′ of its sequence.

In a particular embodiment of this second aspect, said capture probe is linked to at least one attachment molecule positioned 5′ from its sequence and said signal probe is linked to at least one marking molecule positioned 5′ from its sequence.

In another particular embodiment of this second aspect, said capture probe is linked to at least one attachment molecule positioned 5′ from its sequence and said signal probe is linked to at least one marking molecule positioned 3′ from its sequence.

In another particular embodiment of this second aspect, said capture probe is linked to at least one attachment molecule positioned 3′ from its sequence and said signal probe is linked to at least one marking molecule positioned 5′ from its sequence.

In another particular embodiment of this second aspect, said capture probe is linked to at least one attachment molecule positioned 3′ from its sequence and said signal probe is linked to at least one marking molecule positioned 3′ from its sequence.

In all embodiments of this second aspect, the attachment molecule can be selected from a biotin, avidin, streptavidin molecule, a thiol group, an amine group and a carbon.

In a particularly preferred embodiment, the attachment molecule is a biotin molecule.

In all embodiments of this second aspect, the marker molecule can be chosen from a fluorochrome, a biotin, a biotin-bound molecule, digoxigenin, an enzyme using a chemiluminescent substrate, an enzyme using a chromogenic substrate or an enzyme using an electrochemically oxidised substrate.

In a particularly preferred embodiment, the marking molecule is digoxigenin.

According to this second aspect of the invention, said fluorochrome can be selected from the group consisting of: Alexa fluor, in particular Alexa fluor 350, 405, 430, 488, 500, 514, 532, 546, 555, 568, 594, 610, 633, 647, 660, 680, 700, 750 or 790, Fluorescein Isothiocyanate (FITC), Rhodamine, Allophycocyanine (APC) and Phycoerythrin (PE).

According to this second aspect of the invention, said enzyme using a chemiluminescent substrate may be horseradish peroxidase (HRP) and said chemiluminescent substrate may be luminol, or said enzyme using a chemiluminescent substrate may be luciferase and said chemiluminescent substrate may be luciferin.

According to this second aspect of the invention, said enzyme using a chromogenic substrate may be alkaline phosphatase and said chromogenic substrate may be Tetrazolium Nitrobleu (NBT) or Bromochlorylindolophosphate (BCIP), said enzyme using a chromogenic substrate may be horseradish peroxidase (HRP) and said chromogenic substrate may be selected from 3,3′-Diaminobenzidine (DAB), 3,3′,5,5′-Tetramethylbenzidine (TMB), or 2,2′-azino-bis(3-ethylbenzothiazoline-6-sulphonic acid) (ABTS).

According to this second aspect of the invention, said enzyme using an electrochemically oxidizable substrate can be horseradish peroxidase (HRP) and said electrochemically oxidizable substrate can be 3,3′,5,5′-Tetramethylbenzidine (TMB).

In a third aspect, the invention concerns probes for the detection of active living cells of toxic algae.

In this third aspect, the invention concerns at least one probe for the detection of active living cells of toxic algae of the genus Alexandrium, said probe having a sequence selected from the sequences SEQ ID NO: 1, SEQ ID NO: 2, SEQ ID NO: 3, SEQ ID NO: 4, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28 or the sequence of said probe having at least 92% identity with the aforementioned sequences SEQ ID NO: 1, SEQ ID NO: 2, SEQ ID NO: 3, SEQ ID NO: 4, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28.

In this third aspect, the invention also concerns at least one probe for the detection of active living cells of toxic algae of the genus Dinophysis, said probe having a sequence selected from the sequences SEQ ID NO: 30, SEQ ID NO: 31, SEQ ID NO: 33, SEQ ID NO: 35, SEQ ID NO: 37, SEQ ID NO: 38, SEQ ID NO: 39, SEQ ID NO: 41, SEQ ID NO: 42, SEQ ID NO: 44, SEQ ID NO: 45, SEQ ID NO: 47, SEQ ID NO: 48 or the sequence of said probe having at least 92% identity with the abovementioned sequences SEQ ID NO: 30, SEQ ID NO: 31, SEQ ID NO: 33, SEQ ID NO: 35, SEQ ID NO: 37, SEQ ID NO: 38, SEQ ID NO: 39, SEQ ID NO: 41, SEQ ID NO: 42, SEQ ID NO: 44, SEQ ID NO: 45, SEQ ID NO: 47, SEQ ID NO: 48.

In this third aspect, the invention also concerns at least one probe for the detection of active living cells of toxic algae of the genus Pseudo-nitzschia, said probe having a sequence selected from the sequences SEQ ID NO: 49, SEQ ID NO: 51, SEQ ID NO: 52, SEQ ID NO: 54, SEQ ID NO: 55, SEQ ID NO: 57, SEQ ID NO: 58, SEQ ID NO: 60, SEQ ID NO: 61 or the sequence of said probe having at least 92% identity with the abovementioned sequences SEQ ID NO: 49, SEQ ID NO: 51, SEQ ID NO: 52, SEQ ID NO: 54, SEQ ID NO: 55, SEQ ID NO: 57, SEQ ID NO: 58, SEQ ID NO: 60, SEQ ID NO: 61.

In this third aspect, the invention also concerns at least one probe for the detection of active living cells of toxic algae of the genus Prorocentrum, said probe having a sequence selected from the sequences SEQ ID NO: 63, SEQ ID NO: 64, SEQ ID NO: 66, SEQ ID NO: 67, SEQ ID NO: 69, SEQ ID NO: 70, SEQ ID NO: 72, SEQ ID NO: 73 or the sequence of said probe having at least 92% identity with the abovementioned sequences SEQ ID NO: 63, SEQ ID NO: 64, SEQ ID NO: 66, SEQ ID NO: 67, SEQ ID NO: 69, SEQ ID NO: 70, SEQ ID NO: 72, SEQ ID NO: 73.

In this third aspect, the invention also concerns at least one probe for the detection of active living cells of toxic algae of the genus Chattonella, said probe having a sequence selected from the sequences SEQ ID NO: 75, SEQ ID NO: 76, SEQ ID NO: 77, SEQ ID NO: 79, SEQ ID NO: 80, SEQ ID NO: 82 or the sequence of said probe having at least 92% identity with said sequences SEQ ID NO: 75, SEQ ID NO: 76, SEQ ID NO: 77, SEQ ID NO: 79, SEQ ID NO: 80, SEQ ID NO: 82.

In this third aspect, the invention also concerns at least one probe for the detection of active living cells of toxic algae of the genus Gymnodinium, said probe having a sequence selected from the sequences SEQ ID NO: 84, SEQ ID NO: 85, SEQ ID NO: 87, SEQ ID NO: 88, SEQ ID NO: 90, SEQ ID NO: 91, SEQ ID NO: 93, SEQ ID NO: 94 or the sequence of said probe having at least 92% identity with the abovementioned sequences SEQ ID NO: 84, SEQ ID NO: 85, SEQ ID NO: 87, SEQ ID NO: 88, SEQ ID NO: 90, SEQ ID NO: 91, SEQ ID NO: 93, SEQ ID NO: 94.

In this third aspect, the invention also concerns at least one probe for the detection of active living cells of toxic algae of the genus Karenia, said probe having a sequence selected from the sequences SEQ ID NO: 96, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 109, SEQ ID NO: 111, SEQ ID NO: 112, SEQ ID NO: 114, SEQ ID NO: 115, SEQ ID NO: 117, SEQ ID NO: 118 or the sequence of said probe having at least 92% identity with the abovementioned sequences SEQ ID NO: 96, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 109, SEQ ID NO: 111, SEQ ID NO: 112, SEQ ID NO: 114, SEQ ID NO: 115, SEQ ID NO: 117, SEQ ID NO: 118.

In this third aspect, the invention also concerns at least one probe for the detection of active living cells of toxic algae of the genus Lingulodinium, said probe having a sequence selected from the sequences SEQ ID NO: 120, SEQ ID NO: 121, SEQ ID NO: 123, SEQ ID NO: 124, SEQ ID NO: 126, SEQ ID NO: 127 or the sequence of said probe having at least 92% identity with the abovementioned sequences SEQ ID NO: 120, SEQ ID NO: 121, SEQ ID NO: 123, SEQ ID NO: 124, SEQ ID NO: 126, SEQ ID NO: 127.

In this third aspect, the invention also concerns at least one probe for the detection of active living cells of toxic algae of the genus Heterosigma, said probe having a sequence selected from the sequences SEQ ID NO: 128 or the sequence of said probe having at least 92% identity with the above said sequences SEQ ID NO: 128.

According to all embodiments of this third aspect of the invention, said probe is linked to at least one attachment molecule at 3′ or 5′ of its sequence or to at least one marking molecule at 3′ or 5′ of its sequence.

In one particular embodiment, said probe is bound to at least one attachment molecule in 3′ of its sequence.

In another particular embodiment, said probe is linked to at least one attachment molecule in 5′ of its sequence.

In another particular embodiment, said probe is linked to at least one marker molecule in 3′ of its sequence.

In another particular embodiment, said probe is linked to at least one marker molecule in 5′ of its sequence.

According to all embodiments of this third aspect of the invention, the “at least one attachment molecule” can be selected from a biotin, avidin, streptavidin molecule, a thiol group, an amine group and a carbon.

In a particularly preferred embodiment, the “at least one attachment molecule” is a biotin molecule.

According to all embodiments of this third aspect of the invention, the “at least one marker molecule” may be selected from a fluorochrome, a biotin, a biotin-bound molecule, digoxigenin, an enzyme using a chemiluminescent substrate, an enzyme using a chromogenic substrate or an enzyme using an electrochemically oxidised substrate.

In a particularly preferred embodiment, the “at least one marker molecule” is digoxigenin.

According to this third aspect of the invention, said fluorochrome can be selected from the group consisting of: Alexa fluor, in particular Alexa fluor 350, 405, 430, 488, 500, 514, 532, 546, 555, 568, 594, 610, 633, 647, 660, 680, 700, 750 or 790, Fluorescein Isothiocyanate (FITC), Rhodamine, Allophycocyanine (APC) and Phycoerythrin (PE).

According to this third aspect of the invention, said enzyme using a chemiluminescent substrate may be horseradish peroxidase (HRP) and said chemiluminescent substrate may be luminol, or said enzyme using a chemiluminescent substrate may be luciferase and said chemiluminescent substrate may be luciferin.

According to this third aspect of the invention, said enzyme using a chromogenic substrate may be alkaline phosphatase and said chromogenic substrate may be Tetrazolium Nitrobleu (NBT) or Bromochlorylindolophosphate (BCIP), said enzyme using a chromogenic substrate may be horseradish peroxidase (HRP) and said chromogenic substrate may be selected from 3,3′-Diaminobenzidine (DAB), 3,3′,5,5′-Tetramethylbenzidine (TMB), or 2,2′-azino-bis(3-ethylbenzothiazoline-6-sulphonic acid) (ABTS).

According to this third aspect of the invention, said enzyme using an electrochemically oxidisable substrate may be horseradish peroxidase (HRP) and said electrochemically oxidisable substrate may be 3,3′,5,5′-Tetramethylbenzidine (TMB).

In a fourth aspect, the invention relates to a method for the detection of active living cells of toxic algae.

### Embodiment A

Thus, the present invention also concerns a method for detecting active living cells of toxic algae in a sample likely to contain at least one toxic alga of the genus Alexandrium comprising the following steps:
a) optional hybridization resulting from the contact of the said sample with a capture probe and a signal probe specific to toxic algae of the genus Alexandrium, the capture probe and the signal probe forming a pair of probes, the sequences of the said pair of probes being chosen from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22),
   (SEQ ID NO: 23, SEQ ID NO: 24 or SEQ ID NO: 25),
   (SEQ ID NO: 26, SEQ ID NO: 27 or SEQ ID NO: 28)
   x being 2 or 3,
   or the sequences of said probes having at least 92% identity with said sequences SEQ ID NO: 1 to SEQ ID NO: 28.
   said capture probe and said signal probe being capable of hybridizing with the ribosomal nucleic acid of a toxic algae of the genus Alexandrium optionally present in said sample to form a complex
b) detection of said optional complex
   Hybridization indicating the presence of toxic algae of the genus Alexandrium, the minimum detection threshold of the toxic algae of the genus Alexandrium being
      from 100 to 500 active living cells per litre of sample (cells/L) and in particular less than 200 active living cells per litre of sample (cells/L), or
      less than or equal to 0.10 ng RNA per litre of sample and in particular from 0.01 to 0.09 ng RNA per litre of sample.

As mentioned above, a minimum detection limit for the toxic algae of the genus Alexandrium of less than 200 active living cells per litre of sample (cells/L) also corresponds to a minimum detection limit of 0.01 ng to 0.09 ng RNA per litre of sample.

The invention also relates to a method for detecting active living cells of toxic algae in a sample likely to contain at least one toxic alga of the genus Alexandrium, as described above, in which the duration of the implementation of the said detection method is less than one hour.

Thus, the invention also concerns a method for detecting active living cells of toxic algae in a sample likely to contain at least one toxic alga of the genus Alexandrium comprising the following steps:
a) optional hybridization resulting from the contact of the said sample with a capture probe and a signal probe specific to toxic algae of the genus Alexandrium, the capture probe and the signal probe forming a pair of probes, the sequences of the said pair of probes being chosen from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22),
   (SEQ ID NO: 23, SEQ ID NO: 24 or SEQ ID NO: 25),
   (SEQ ID NO: 26, SEQ ID NO: 27 or SEQ ID NO: 28)
   x being 2 or 3,
   or the sequences of said probes having at least 92% identity with said sequences SEQ ID NO: 1 to SEQ ID NO: 28.
   said capture probe and said signal probe being capable of hybridizing with the ribosomal nucleic acid of a toxic algae of the genus Alexandrium optionally present in said sample to form a complex
b) detection of said optional complex
   Hybridization indicating the presence of toxic algae of the genus Alexandrium, the duration of the implementation of the said detection method being less than one hour.

In this embodiment, the invention also concerns a method for detecting active living cells of toxic algae in a sample likely to contain at least one toxic alga of the genus Alexandrium, as described above, in which the minimum detection threshold of the toxic alga of the genus Alexandrium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample,
   and the duration of the implementation of the said detection method is less than one hour

In a particular embodiment, the method for detecting active live cells of toxic algae in a sample likely to contain at least one toxic alga of the genus Alexandrium, as described above, may also include, before the optional hybridization step, a step of preparation of the said sample to be analysed in order to obtain a prepared sample.

In a particular embodiment, the method for the detection of active living cells of toxic algae in a sample likely to contain at least one toxic alga of the genus Alexandrium, as described above, may also include a step for the quantification of toxic algae of the genus Alexandrium in the case of hybridization indicating the presence of toxic algae of the genus Alexandrium.

Thus, the invention also concerns a method for detecting active living cells of toxic algae in a sample likely to contain at least one toxic alga of the genus Alexandrium comprising the following steps:
a) preparation of said sample to be analysed in order to obtain a prepared sample
b) optional hybridization resulting from the contact of the said prepared sample with a capture probe and a signal probe specific to toxic algae of the genus Alexandrium, the capture probe and the signal probe forming a pair of probes, the sequences of the said pair of probes being chosen from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22),
   (SEQ ID NO: 23, SEQ ID NO: 24 or SEQ ID NO: 25),
   (SEQ ID NO: 26, SEQ ID NO: 27 or SEQ ID NO: 28)
   x being 2 or 3,
   or the sequences of said probes having at least 92% identity with said sequences SEQ ID NO: 1 to SEQ ID NO: 28.
   said capture probe and said signal probe being capable of hybridizing with the ribosomal nucleic acid of a toxic algae of the genus Alexandrium optionally present in said sample to form a complex
c) detection of said optional complex
d) quantification of toxic algae of the genus Alexandrium, in the case of hybridization
   Hybridization indicating the presence of toxic algae of the genus Alexandrium,
   the minimum detection threshold of the toxic algae of the genus Alexandrium being
      from 100 to 500 active living cells per litre of sample (cells/L) and in particular less than 200 active living cells per litre of sample (cells/L), or
      less than or equal to 0.10 ng RNA per litre of sample and in particular from 0.01 to 0.09 ng RNA per litre of sample.

As mentioned above, a minimum detection limit for the toxic algae of the genus Alexandrium of less than 200 active living cells per litre of sample (cells/L) also corresponds to a minimum detection limit of 0.01 ng to 0.09 ng RNA per litre of sample.

The invention also concerns a method for detecting active living cells of toxic algae in a sample likely to contain at least one toxic alga of the genus Alexandrium comprising the following steps:
a) preparation of said sample to be analysed in order to obtain a prepared sample
b) optional hybridization resulting from the contact of the said prepared sample with a capture probe and a signal probe specific to toxic algae of the genus Alexandrium, the capture probe and the signal probe forming a pair of probes, the sequences of the said pair of probes being chosen from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22),
   (SEQ ID NO: 23, SEQ ID NO: 24 or SEQ ID NO: 25),
   (SEQ ID NO: 26, SEQ ID NO: 27 or SEQ ID NO: 28)
   x being 2 or 3,
   or the sequences of said probes having at least 92% identity with said sequences SEQ ID NO: 1 to SEQ ID NO: 28.
   said capture probe and said signal probe being capable of hybridizing with the ribosomal nucleic acid of a toxic algae of the genus Alexandrium optionally present in said sample to form a complex
c) detection of said optional complex
d) quantification of toxic algae of the genus Alexandrium, in the case of hybridization
   hybridization indicating the presence of toxic algae of the genus Alexandrium the duration of the implementation of steps b) and c) being less than one hour.

In a particular embodiment, the invention relates to a method for the detection of active living cells of toxic algae in a sample likely to contain at least one toxic alga of the genus Alexandrium, as described above, in which the minimum detection threshold of the toxic alga of the genus Alexandrium is less than 200 active living cells per litre of sample (cells/L) or less than or equal to 0.10 ng RNA per litre of sample and the implementation of steps b) and c) is less than one hour.

Thus, the invention also concerns a method for detecting active living cells of toxic algae in a sample likely to contain at least one toxic alga of the genus Alexandrium comprising the following steps:
a) preparation of said sample to be analysed in order to obtain a prepared sample
b) optional hybridization resulting from the contact of the said prepared sample with a capture probe and a signal probe specific to toxic algae of the genus Alexandrium, the capture probe and the signal probe forming a pair of probes, the sequences of the said pair of probes being chosen from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22),
   (SEQ ID NO: 23, SEQ ID NO: 24 or SEQ ID NO: 25),
   (SEQ ID NO: 26, SEQ ID NO: 27 or SEQ ID NO: 28)
   x being 2 or 3,
   or the sequences of said probes having at least 92% identity with said sequences SEQ ID NO: 1 to SEQ ID NO: 28,
   said capture probe and said signal probe being capable of hybridizing with the ribosomal nucleic acid of a toxic algae of the genus Alexandrium optionally present in said sample to form a complex
c) detection of said optional complex
d) quantification of toxic algae of the genus Alexandrium, in the case of hybridization,
   Hybridization indicating the presence of toxic algae of the genus Alexandrium,
   the minimum detection threshold of the toxic algae of the genus Alexandrium being
      from 100 to 500 active living cells per litre of sample (cells/L) and in particular less than 200 active living cells per litre of sample (cells/L), or
      less than or equal to 0.10 ng RNA per litre of sample and in particular from 0.01 to 0.09 ng RNA per litre of sample,
   the duration of the implementation of steps b) and c) being less than one hour.

In a particular embodiment, the invention concerns a method of detecting active living cells of toxic algae in a sample likely to contain at least one toxic alga of the genus Alexandrium as described above, in which said step of preparing said sample to be analysed comprises the following steps:
a) a step of concentration of the sample in order to obtain a concentrated sample
b) a step of lysis of the toxic algae optionally present in the said sample, resulting in the release of ribosomal nucleic acids from the toxic algae of the genus Alexandrium likely to be contained in the said sample.

In a particular embodiment, the invention concerns a method for detecting active living cells of toxic algae in a sample likely to contain at least one toxic alga of the genus Alexandrium as described above, which may furthermore comprise a step of extraction and purification of the ribosomal nucleic acids obtained following the lysis step b) using a nucleic acid extraction and purification protocol known to man of the art.

In a particular embodiment, the invention concerns a method for detecting active living cells of toxic algae in a sample likely to contain at least one toxic alga of the genus Alexandrium as described above, which may furthermore comprise a step of fragmentation of the ribosomal nucleic acids obtained following the lysis step b) in order to homogenise the size of the extracted and purified nucleic acids.

In a particular embodiment, the said step of concentrating the said sample may be a centrifugation or filtration step.

In a particular embodiment, the said filtration can be carried out on nylon or polycarbonate filters. These filters can, for example, have a porosity of 0.2 to 100 μm.

In a particular embodiment, the said lysis step may be a chemical lysis step comprising the addition of a lysis solution to the said concentrated sample obtained in step (a) described above.

In a particular embodiment, said lysis solution may comprise a neutral buffer, a chaotropic agent, an ionic or non-ionic detergent, a reducing agent and a chelating agent.

The neutral buffer can for example be phosphate, Saline Sodium Citrate (SSC) or Tris. The chaotropic agent can for example be guanidium chloride. The ionic or non-ionic detergent can for example be Sodium Dodecyl Sulphate (SDS) or Triton® X100. The reducing agent can for example be b-mercaptoethanol or DiThioTreitol. The chelating agent can for example be Ethylene Diamine Tetra Acetic Acid (EDTA) or Ethylene Glycol Tetra Acetic Acid (EGTA).

In a particular embodiment, the said chemical lysis step may be accompanied by thermal lysis, sonic lysis and/or mechanical lysis. Thermal lysis can for example be carried out with liquid nitrogen or by heating the sample. Sonic lysis can for example be carried out using ultrasound or vibration. Mechanical lysis can for example be carried out using a vortex or grinding.

In a particular embodiment, the invention concerns a method for detecting active living cells of toxic algae in a sample likely to contain at least one toxic alga of the genus Alexandrium as described above, in which said capture probe is linked to at least one attachment molecule positioned 5′ from its sequence and said signal probe is linked to at least one labelling molecule positioned 5′ from its sequence.

In another particular embodiment, the invention concerns a method for detecting active living cells of toxic algae in a sample likely to contain at least one toxic alga of the genus Alexandrium as described above, in which said capture probe is linked to at least one attachment molecule positioned 5′ from its sequence and said signal probe is linked to at least one labelling molecule positioned 3′ from its sequence.

In another particular embodiment, the invention concerns a method for detecting active living cells of toxic algae in a sample likely to contain at least one toxic alga of the genus Alexandrium as described above, in which said capture probe is linked to at least one attachment molecule positioned 3′ of its sequence and said signal probe is linked to at least one labelling molecule positioned 5′ of its sequence.

In another particular embodiment, the invention relates to a method for detecting active living cells of toxic algae in a sample likely to contain at least one toxic alga of the genus Alexandrium as described above in which said capture probe is linked to at least one attachment molecule positioned 3′ of its sequence and said signal probe is linked to at least one labelling molecule positioned 3′ of its sequence.

In a particular embodiment, the attachment molecule can be selected from a biotin, avidin, streptavidin molecule, a thiol group, an amine group and a carbon group.

In a particularly preferred embodiment, the said attachment molecule is a biotin molecule.

In a particular embodiment, the marking molecule may be chosen from a fluorochrome, a biotin, a biotin-bound molecule, digoxigenin, an enzyme using a chemiluminescent substrate, an enzyme using a chromogenic substrate or an enzyme using an electrochemical oxidation substrate.

In a particularly preferred embodiment, the marking molecule is digoxigenin.

The invention relates to a detection method as described above, wherein said capture probe is linked to at least one attachment molecule positioned 5′ of its sequence and said signal probe is linked to at least one marking molecule positioned 5′ of its sequence, or

said capture probe is linked to at least one attachment molecule positioned 5′ of its sequence and said signal probe is linked to at least one marker molecule positioned 3′ of its sequence, or
said capture probe is linked to at least one attachment molecule positioned 3′ of its sequence and said signal probe is linked to at least one marker molecule positioned 5′ of its sequence, or
said capture probe is linked to at least one attachment molecule positioned 3′ of its sequence and said signal probe is linked to at least one labelling molecule positioned 3′ of its sequence, said “at least one attachment molecule” being in particular selected from a biotin molecule, avidin, streptavidin, a thiol group, an amine group and a carbon group, preferably a biotin molecule,
the said “at least one marking molecule” being chosen in particular from a fluorochrome, a biotin, a biotin-bound molecule, digoxigenin, an enzyme using a chemiluminescent substrate, an enzyme using a chromogenic substrate or an enzyme using an electrochemically oxidised substrate, preferably digoxigenin.

In a particular embodiment, said fluorochrome can be selected from the group consisting of: Alexa fluor, in particular Alexa fluor 350, 405, 430, 488, 500, 514, 532, 546, 555, 568, 594, 610, 633, 647, 660, 680, 700, 750 or 790, Fluorescein Isothiocyanate (FITC), Rhodamine, Allophycocyanine (APC) and Phycoerythrin (PE).

In a particular embodiment, said enzyme using a chemiluminescent substrate may be horseradish peroxidase (HRP) and said chemiluminescent substrate may be luminol, or said enzyme using a chemiluminescent substrate may be luciferase and said chemiluminescent substrate may be luciferin.

In a particular embodiment, said enzyme using a chromogenic substrate may be alkaline phosphatase and said chromogenic substrate may be Tetrazolium Nitroblue (NBT) or Bromochlorylindolophosphate (BCIP), said enzyme using a chromogenic substrate may be horseradish peroxidase (HRP) and said chromogenic substrate may be selected from 3,3′-Diaminobenzidine (DAB), 3,3′,5,5′-Tetramethylbenzidine (TMB), or 2,2′-azino-bis(3-ethylbenzothiazoline-6-sulphonic acid) (ABTS).

In a particular embodiment, said enzyme using an electrochemically oxidizable substrate may be horseradish peroxidase (HRP) and said electrochemically oxidizable substrate may be 3,3′,5,5′-Tetramethylbenzidine (TMB).

In a particular embodiment, the invention relates to a method for detecting active living cells of toxic algae in a sample likely to contain at least one toxic alga of the genus Alexandrium as described above, in which said hybridization can be carried out in a hybridization solution.

In a particular embodiment, said hybridization solution comprises 0 to 0.3 M NaCl, 0 to 0.1 M buffer selected from citrate, Tris-HCl, PIPES, HEPES or phosphate, 0.001 to 0.05% detergent agent selected from SDS, Triton®, TWEEN®20, optionally 0.001 to 0.5 M chelating agent selected from EDTA or EGTA, optionally 0.1 to 30% blocking agent selected from BSA, herring DNA, salmon DNA, calf DNA, yeast DNA or an exogenous protein and optionally another chemical agent selected from MgCl₂, KCl and CaCl₂), preferably MgCl₂.

Alternatively, said hybridization solution comprises 0.1 M to 1 M NaCl or KCl, 0.01 M to 1 M Tris-HCl, HEPES, PBS, KH₂PO₄ or SSC with a pH ranging from 6.0 to 9.0, 0.01 to 0.05% of detergent selected from SDS or N-Lauroylsarcosine, optionally 0.01 and 0.1 M chelating agent selected from EDTA, EGTA or a similar chelating agent selected from calcium citrate or sodium hexametaphosphate and optionally 0.1 and 30% blocking agent selected from a protein such as Bovine Serum Albumin (BSA) or a nucleic acid such as Herring DNA.

In a particularly preferred embodiment, the said hybridization solution consists of 0.3 M NaCl, 0.08 M Tris-HCl and 0.04% SDS and is pH 8.

In a particular embodiment, the said hybridization is carried out at a temperature ranging from 37° C. to 70° C. In a particularly preferred embodiment, said hybridization is carried out at a temperature of 60° C.

In a particular embodiment, the contact time of said sample with said capture probe and said signal probe is between 10 and 60 minutes. In a particularly preferred embodiment, the contact time of said sample with said capture probe and said signal probe is 10 minutes.

In a particular embodiment, the said detection step may be followed by one or more washing steps with a washing solution. In a particularly preferred embodiment, three washing steps are performed.

In a particular embodiment, each washing step can last from 1 to 60 minutes.

In a particular embodiment, said washing solution comprises 0 to 0.3 M NaCl, 0 to 0.1 M buffer selected from citrate, Tris-HCl, PIPES, HEPES or phosphate, 0.001 to 0.05% detergent agent selected from SDS, Triton®, TWEEN®20, optionally 0.001 to 0.5 M chelating agent selected from EDTA or EGTA, optionally 0.1 to 30% blocking agent selected from BSA, herring DNA, salmon DNA, calf DNA, yeast DNA or an exogenous protein and optionally another chemical agent selected from MgCl₂, KCl and CaCl₂), preferably MgCl₂.

Alternatively, said washing solution comprises 0.1 M to 1 M of NaCl or KCl, 0.01 M to 1 M of Tris-HCl, HEPES, PBS, KH₂PO₄ or SSC with a pH ranging from 6.0 to 9.0, 0.01 and 0.05% of detergent agent selected from SDS or N-Lauroylsarcosine, optionally 0.01 and 0.1 M chelating agent selected from EDTA, EGTA or a similar chelating agent selected from calcium citrate or sodium hexametaphosphate and optionally 0.1 and 30% blocking agent selected from a protein such as Bovine Serum Albumin Protein (BSA) or a nucleic acid such as Herring DNA.

In a particularly preferred embodiment, said wash solution comprises 0.01 and 0.7 M of PBS, Na₂HPO₄, KH₂PO₄, K₂PO₄ and/or SSC, and 0.1 and 0.4 M of NaCl or KCl.

In a particularly preferred embodiment, the said washing solution consists of 0.1M K₂PO₄, 0.1M KH₂PO₄ and 0.1M KCl and has a pH of 7.6.

In a particular embodiment, the invention also concerns a method for the detection of active living cells of toxic algae in a sample likely to contain at least one toxic alga of the genus Alexandrium as described above, in which the said step of detecting the said complex can be carried out by a fluorescence reader, or luminescence reader, by an absorbance reader, by a gamma camera, by a beta camera or by means of an ammeter or a potentiometer.

The said detection step of the said complex can be carried out by fluorescence microscopy or fluorescence reader when:
the marking molecule is a fluorochrome
when the marker molecule is biotin and is detected via a fluorochrome conjugated to streptavidin or avidin
when the marker molecule is conjugated to biotin and is detected via a fluorochrome conjugated to streptavidin or avidin
when the labelling molecule is digoxigenin and is detected via a fluorochrome conjugated to an anti-digoxigenin antibody.

The said detection step of the said complex can be performed by luminescence reader when:
the marking molecule is an enzyme using a chemiluminescent substrate
the marker molecule is biotin and is detected via an enzyme using a chemiluminescent substrate conjugated to streptavidin or avidin
the marker molecule is conjugated to biotin and is detected via an enzyme using a chemiluminescent substrate conjugated to streptavidin or avidin
the marker molecule is digoxigenin and is detected via an enzyme using a chemiluminescent substrate conjugated to an anti-digoxigenin antibody.

The said detection step of the said complex can be carried out by an absorbance reader when:
the marking molecule is an enzyme using a chromogenic substrate
the marker molecule is biotin and is detected via an enzyme using a chromogenic substrate conjugated to streptavidin or avidin
the marker molecule is conjugated to biotin and is detected via an enzyme using a chromogenic substrate conjugated to streptavidin or avidin
the marker molecule is digoxigenin and is detected via an enzyme using a chromogenic substrate conjugated to an anti-digoxigenin antibody.

The said detection step of the said complex can be carried out with the help of an ammeter or potentiometer when:
the marking molecule is an enzyme using an electrochemical oxidation substrate
the marker molecule is biotin and is detected via an enzyme using a substrate with electrochemical oxidation conjugated to streptavidin or avidin
the marker molecule is conjugated to biotin and is detected via an enzyme using an electrochemically oxidised substrate conjugated to streptavidin or avidin
the marker molecule is digoxigenin and is detected via an enzyme using a substrate with electrochemical oxidation conjugated to an anti-digoxigenin antibody,
   said enzyme using an electrochemically oxidizing substrate reacting in the presence of _{H2O2} and oxidizing said electrochemically oxidizing substrate which, when reduced, then generates an electric potential difference measured by the electrode.

In one embodiment, the said florochrome can be chosen from the group consisting of: Alexa fluor, in particular Alexa fluor 350, 405, 430, 488, 500, 514, 532, 546, 555, 568, 594, 610, 633, 647, 660, 680, 700, 750 or 790, Fluorescein Isothiocyanate (FITC), Rhodamine, Allophycocyanine (APC) and Phycoerythrin (PE).

In one embodiment, said enzyme using a chemiluminescent substrate may be horseradish peroxidase (HRP) and said chemiluminescent substrate may be luminol, or said enzyme using a chemiluminescent substrate may be luciferase and said chemiluminescent substrate may be luciferin.

In one embodiment, said enzyme using a chromogenic substrate may be alkaline phosphatase and said chromogenic substrate may be Tetrazolium Nitroblue (NBT) or Bromochlorylindolophosphate (BCIP), or said enzyme using a chromogenic substrate may be horseradish peroxidase (HRP) and said chromogenic substrate may be selected from 3,3′-Diaminobenzidine (DAB), 3,3′,5,5′-Tetramethylbenzidine (TMB), or 2,2′-azino-bis(3-ethylbenzothiazoline-6-sulphonic acid) (ABTS).

In one of embodiment, said enzyme using an electrochemically oxidizable substrate may be horseradish peroxidase (HRP) and said electrochemically oxidizable substrate may be 3,3′,5,5′-Tetramethylbenzidine (TMB).

For the quantification step, the results can be expressed as absorbance at 630 nm or 450 nm after reading with a microplate reader, or as current intensity after reading with an ammeter or potentiometer. For each type of toxic algae to be detected, a calibration curve is produced using synthetic standards of known increasing concentrations. The quantification is determined by relating the average absorbance or current values from each sample to the calibration curve. The standards are related to RNA or cell equivalent values established from known numbers of cultured cells added to an uncontaminated environmental sample.

As indicated above, according to the present invention, the limit of quantification of the algae listed above is 0.04 to 0.12 ng RNA per litre of sample depending on the type of algae.

According to this embodiment, the sample may be a sample of seawater, brackish water, culture media or microalgae cultures produced for commercial purposes.

### Embodiment B

The invention also relates to a method for detecting active living cells of toxic algae, as described above in embodiment A, in a sample likely to contain in addition at least one toxic algae of the genus Dinophysis, comprising in addition to the optional hybridization step resulting from bringing said sample into contact with a capture probe and a signal probe specific to toxic algae of the genus Alexandrium,

an optional hybridization step resulting from bringing said sample into contact with a capture probe and a signal probe specific to toxic algae of the genus Dinophysis, the capture probe and the signal probe forming a pair of probes, the sequences of said pair of probes being chosen from x elements of one of the following sets:
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44), or
(SEQ ID NO: 46, SEQ ID NO: 47 or SEQ ID NO: 48)
   x being 2 or 3,
   or the sequences of said probes having at least 92% identity with said sequences SEQ ID NO: 29 to SEQ ID NO: 48.

Hybridization indicating the presence of toxic algae of the genus Dinophysis.

All the different embodiments described for the detection method in embodiment A can be used for embodiment B.

As previously for the detection of Alexandrium according to embodiment A, in a particular embodiment, the minimum detection threshold of the toxic algae of the genus Dinophysis is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment C

In the same way, the invention also concerns one of the methods for the detection of active living cells of toxic algae, one of the methods for the detection of active living cells of toxic algae, as described above according to embodiment A or according to embodiment B, in a sample likely to contain in addition at least one toxic algae of the genus Pseudo-Nitzschia, comprising in addition to the optional hybridization step resulting from the bringing of the said sample into contact with a capture probe and a signal probe specific to toxic algae of the genus Alexandrium,

an optional hybridization step resulting from bringing said sample into contact with a capture probe and a signal probe specific to toxic algae of the genus Pseudo-nitzschia, the capture probe and the signal probe forming a probe pair, the sequences of said probe pair being selected from x elements of one of the following sets:
(SEQ ID NO: 49 and SEQ ID NO: 50)
(SEQ ID NO: 51 and SEQ ID NO: 52)
(SEQ ID NO: 53, SEQ ID NO: 54 or SEQ ID NO: 55)
(SEQ ID NO: 56, SEQ ID NO: 57 or SEQ ID NO: 58), or
(SEQ ID NO: 59, SEQ ID NO: 60 or SEQ ID NO: 61)
   x being 2 or 3,
   or the sequences of said probes having at least 92% identity with said sequences SEQ ID NO: 49 to SEQ ID NO: 61,
   hybridization indicating the presence of toxic algae of the genus Pseudo nitzschia.

All the different embodiments described for the detection method in embodiment A can be used for embodiment C.

As previously for the detection of Alexandrium according to embodiment A, in a particular embodiment, the minimum detection threshold of the toxic algae of the genus Pseudo-nitzschia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment D

In the same way, the invention also concerns one of the methods for the detection of active living cells of toxic algae, one of the methods for the detection of active living cells of toxic algae, as described above according to embodiments A, B or C, in a sample likely to contain in addition at least one toxic algae of the genus Prorocentrum, comprising in addition to the optional hybridization step resulting from bringing said sample into contact with a capture probe and a signal probe specific to toxic algae of the genus Alexandrium,

an optional hybridization step resulting from bringing said sample into contact with a capture probe and a signal probe specific to toxic algae of the genus Prorocentrum, the capture probe and the signal probe forming a pair of probes, the sequences of said pair of probes being chosen from x elements of one of the following sets:
(SEQ ID NO: 62, SEQ ID NO: 63 or SEQ ID NO: 64)
(SEQ ID NO: 65, SEQ ID NO: 66 or SEQ ID NO: 67)
(SEQ ID NO: 68, SEQ ID NO: 69 or SEQ ID NO: 70)
(SEQ ID NO: 71, SEQ ID NO: 72 or SEQ ID NO: 73)
   x being 2 or 3,
   or the sequences of the said probes having at least 92% identity with the abovementioned sequences SEQ ID NO: 62 to SEQ ID NO: 73.

Hybridization indicating the presence of toxic algae of the genus Prorocentrum.

All the different embodiments described for the detection method in embodiment A can be used for embodiment D.

As previously for the detection of Alexandrium according to embodiment A, in a particular embodiment, the minimum detection threshold of the toxic algae of the genus Prorocentrum is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment E

In the same way, the invention also concerns one of the methods for detecting active living cells of toxic algae, as described above according to embodiments A, B, C, or D, in a sample likely to contain in addition at least one toxic algae of the genus Chattonella, comprising in addition to the optional hybridization step resulting from bringing said sample into contact with a capture probe and a signal probe specific to toxic algae of the genus Alexandrium, an optional hybridization step resulting from bringing said sample into contact with a capture probe and a signal probe specific to toxic algae of the genus Chattonella, the capture probe and the signal probe forming a pair of probes, the sequences of said pair of probes being chosen from x elements of one of the following sets:
(SEQ ID NO: 74, SEQ ID NO: 75 or SEQ ID NO: 76)
(SEQ ID NO: 77, SEQ ID NO: 78 or SEQ ID NO: 79)
(SEQ ID NO: 80, SEQ ID NO: 81 or SEQ ID NO: 82)
   x being 2 or 3,
   or the sequences of said probes having at least 92% identity with said sequences SEQ ID NO: 74 to SEQ ID NO: 82.

Hybridization indicating the presence of toxic algae of the genus Chattonella.

All the different types embodiment described for the detection method in embodiment A can be used for embodiment E.

As previously for the detection of Alexandrium according to embodiment A, in a particular embodiment, the minimum detection threshold of the toxic algae of the genus Chattonella is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment F

In the same way, the invention also concerns one of the methods for the detection of active living cells of toxic algae, one of the methods for the detection of active living cells of toxic algae, as described above according to embodiments A, B, C, D or E, in a sample likely to contain in addition at least one toxic algae of the genus Gymnodinium, comprising in addition to the optional hybridization step resulting from bringing said sample into contact with a capture probe and a signal probe specific to toxic algae of the genus Alexandrium, an optional hybridization step resulting from bringing said sample into contact with a capture probe and a signal probe specific to toxic algae of the genus Gymnodinium, the capture probe and the signal probe forming a pair of probes, the sequences of said pair of probes being chosen from x elements of one of the following sets:
(SEQ ID NO: 83, SEQ ID NO: 84 or SEQ ID NO: 85)
(SEQ ID NO: 86, SEQ ID NO: 87 or SEQ ID NO: 88)
(SEQ ID NO: 89, SEQ ID NO: 90 or SEQ ID NO: 91)
(SEQ ID NO: 92, SEQ ID NO: 93 or SEQ ID NO: 94)
   x being 2 or 3,
   or the sequences of said probes having at least 92% identity with the above-mentioned sequences SEQ ID NO: 83 to SEQ ID NO: 94.

Hybridization indicating the presence of toxic algae of the genus Gymnodinium.

All the different types embodiment described for the detection method in embodiment A can be used for embodiment F.

As previously for the detection of Alexandrium according to embodiment A, in a particular embodiment, the minimum detection threshold of the toxic algae of the genus Gymnodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment G

In the same way, the invention also concerns one of the methods for the detection of active living cells of toxic algae, one of the methods for the detection of active living cells of toxic algae, as described above according to embodiments A, B, C, D, E or F, in a sample likely to contain in addition at least one toxic algae of the genus Karenia, comprising in addition to the optional hybridization step resulting from bringing said sample into contact with a capture probe and a signal probe specific to toxic algae of the genus Alexandrium,

an optional hybridization step resulting from bringing said sample into contact with a capture probe and a signal probe specific to toxic algae of the genus Karenia, the capture probe and the signal probe forming a pair of probes, the sequences of said pair of probes being selected from x elements of one of the following sets:
(SEQ ID NO: 95, SEQ ID NO: 96 or SEQ ID NO: 97)
(SEQ ID NO: 98, SEQ ID NO: 99 or SEQ ID NO: 100)
(SEQ ID NO: 101, SEQ ID NO: 102 or SEQ ID NO: 103)
(SEQ ID NO: 104, SEQ ID NO: 105 or SEQ ID NO: 106)
(SEQ ID NO: 107, SEQ ID NO: 108 or SEQ ID NO: 109)
(SEQ ID NO: 110, SEQ ID NO: 111 or SEQ ID NO: 112)
(SEQ ID NO: 113, SEQ ID NO: 114 or SEQ ID NO: 115)
(SEQ ID NO: 116, SEQ ID NO: 117 or SEQ ID NO: 118)
   x being 2 or 3,
   or the sequences of said probes having at least 92% identity with said sequences SEQ ID NO: 95 to SEQ ID NO: 118.

Hybridization indicating the presence of toxic algae of the genus Karenia.

All the different embodiments described for the detection method in embodiment A can be used for embodiment G.

As previously for the detection of Alexandrium according to embodiment A, in a particular embodiment, the minimum detection threshold of the toxic algae of the genus Karenia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment H

In the same way, the invention also concerns one of the methods for the detection of active living cells of toxic algae, one of the methods for the detection of active living cells of toxic algae, as described above according to embodiments A, B, C, D, E, F or G in a sample likely to contain in addition at least one toxic algae of the genus Lingulodinium, comprising in addition to the optional hybridization step resulting from bringing said sample into contact with a capture probe and a signal probe specific to toxic algae of the genus Alexandrium,

an optional hybridization step resulting from bringing said sample into contact with a capture probe and a signal probe specific to toxic algae of the genus Lingulodinium, the capture probe and the signal probe forming a pair of probes, the sequences of said pair of probes being chosen from x elements of one of the following sets:
(SEQ ID NO: 119, SEQ ID NO: 120 or SEQ ID NO: 121)
(SEQ ID NO: 122, SEQ ID NO: 123 or SEQ ID NO: 124)
(SEQ ID NO: 125, SEQ ID NO: 126 or SEQ ID NO: 127)
   x being 2 or 3,
   or the sequences of said probes having at least 92% identity with said sequences SEQ ID NO: 119 to SEQ ID NO: 127.

Hybridization indicating the presence of toxic algae of the genus Lingulodinium.

All the different embodiments described for the detection method in embodiment A can be used for embodiment H.

As previously for the detection of Alexandrium according to embodiment A, in a particular embodiment, the minimum detection threshold of the toxic algae of the genus Lingulodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment I

In the same way, the invention also concerns one of the methods for detecting active living cells of toxic algae, as described above according to embodiments A, B, C, D, E, F, G or H, in a sample likely to contain in addition at least one toxic algae of the genus Heterosigma, comprising in addition to the optional hybridization step resulting from bringing said sample into contact with a capture probe and a signal probe specific to toxic algae of the genus Alexandrium,

an optional hybridization step resulting from bringing said sample into contact with a capture probe and a signal probe specific to toxic algae of the genus Heterosigma, the capture probe and the signal probe forming a pair of probes, the sequences of said pair of probes being chosen from x elements of one of the following sets:
(SEQ ID NO: 128 and SEQ ID NO: 129)
   x being 2 or 3,
   or the sequences of said probes having at least 92% identity with said sequences SEQ ID NO: 128 to SEQ ID NO: 129.

Hybridization indicating the presence of toxic algae of the genus Heterosigma.

All the different types embodiment described for the detection method in embodiment A can be used for embodiment I.

As previously for the detection of Alexandrium according to embodiment A, in a particular embodiment, the minimum detection threshold of the toxic algae of the genus Heterosigma is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

As with the first aspect relating to use, all combinations of embodiments A, B, C, D, E, F, G, H and/or I of this fourth aspect can be considered. In this way, all combinations of toxic algae B to 1128 described in the first aspect can be detected by the methods as described in this fourth aspect.

The hybridization step described above between the ribosomal nucleic acid of the toxic algae to be detected and the capture and signal probes can be performed in several ways.

In a first aspect, the capture probes are incubated on the support and the signal probes are incubated with the ribosomal nucleic acids of the toxic algae to be detected. Then, any pairs formed between the signal probes and the ribosomal nucleic acids of the toxic algae to be detected are incubated with the support containing the capture probes.

In a second aspect, the capture probes are incubated on the support. Then, the signal probes, the ribosomal nucleic acids of the toxic algae to be detected and the support containing the capture probes are incubated together.

In a third aspect, the capture and signal probes are incubated with the ribosomal nucleic acids of the toxic algae to be detected. This mixture is then incubated with the carrier.

In a fourth aspect, the capture probes are incubated with the ribosomal nucleic acids of the toxic algae to be detected. This mixture is then incubated with the carrier and the signal probes.

### Embodiment A1

Thus, according to a particular embodiment of this fourth aspect, the invention concerns a method of detecting active living cells of toxic algae in a sample likely to contain at least one toxic alga of the genus Alexandrium, comprising the following steps:
a) addition of an optional complex formed between the ribosomal nucleic acid of a toxic algae of the genus Alexandrium, and a signal probe on a support containing a capture probe,
b) detection of the optional hybridization of the aforementioned complex with the said capture probe, the hybridization taking place between the capture probe and the ribosomal nucleic acid of the aforementioned complex,
   Hybridization indicating the presence of toxic algae of the genus Alexandrium,
   said capture probe and said signal probe forming a probe pair, the sequences of said probe pair being selected from x elements of one of the following sets:
      (SEQ ID NO: 1, SEQ ID NO: 2 or SEQ ID NO: 3)
      (SEQ ID NO: 4 and SEQ ID NO: 5)
      (SEQ ID NO: 6 and SEQ ID NO: 7)
      (SEQ ID NO: 8, SEQ ID NO: 9 or SEQ ID NO: 10)
      (SEQ ID NO: 11, SEQ ID NO: 12 or SEQ ID NO: 13)
      (SEQ ID NO: 14, SEQ ID NO: 15 or SEQ ID NO: 16)
      (SEQ ID NO: 17, SEQ ID NO: 18 or SEQ ID NO: 19)
      (SEQ ID NO: 20, SEQ ID NO: 21 or SEQ ID NO: 22),
      (SEQ ID NO: 23, SEQ ID NO: 24 or SEQ ID NO: 25),
      (SEQ ID NO: 26, SEQ ID NO: 27 or SEQ ID NO: 28)
   x being 2 or 3,
   or the sequences of said probes having at least 92% identity with said sequences SEQ ID NO: 1 to SEQ ID NO: 28.

All the different types embodiment described for the detection procedure in embodiment A can be used for embodiment A1.

As before, in a particular embodiment, the minimum detection threshold of the toxic algae of the genus Alexandrium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment B1

The invention also concerns a method for detecting active living cells of toxic algae as described above according to the A1 embodiment, in a sample likely to contain in addition at least one toxic alga of the genus Dinophysis, comprising, in addition, the addition of an optional complex formed between the ribosomal nucleic acid of a toxic alga of the genus Dinophysis and a signal probe on a support containing a capture probe,

said capture probe and said signal probe forming a probe pair, the sequences of said probe pair being selected from x elements of one of the following sets:
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 3, SEQ ID NO: 44 or SEQ ID NO: 45), or
(SEQ ID NO: 46, SEQ ID NO: 47 or SEQ ID NO: 48)
   x being 2 or 3,
   or the sequences of said probes having at least 92% identity with said sequences SEQ ID NO: 29 to SEQ ID NO: 48
   hybridization indicating the presence of toxic algae of the genus Dinophysis.

All the different types embodiment described for the detection procedure in embodiment A can be used for embodiment B1.

As previously, in a particular embodiment, the minimum detection threshold of the toxic algae of the genus Dinophysis is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment C1

The invention also concerns one of the methods for detecting active living cells of toxic algae as described above according to embodiment A1 or according to embodiment B1, in a sample likely to contain in addition at least one toxic algae of the genus Pseudo-nitzschia, comprising in addition the addition of an optional complex formed between the ribosomal nucleic acid of a toxic algae of the genus Pseudo-nitzschia and a signal probe on a support containing a capture probe,

said capture probe and said signal probe forming a probe pair, the sequences of said probe pair being selected from x elements of one of the following sets:
(SEQ ID NO: 49 and SEQ ID NO: 50)
(SEQ ID NO: 51 and SEQ ID NO: 52)
(SEQ ID NO: 53, SEQ ID NO: 54 or SEQ ID NO: 55)
(SEQ ID NO: 56, SEQ ID NO: 57 or SEQ ID NO: 58), or
(SEQ ID NO: 59, SEQ ID NO: 60 or SEQ ID NO: 61)
   x being 2 or 3,
   or the sequences of said probes having at least 92% identity with said sequences SEQ ID NO: 49 to SEQ ID NO: 61
   hybridization indicating the presence of toxic algae of the genus Pseudo-nitzschia.

All the different types embodiment described for the detection procedure in embodiment A can be applied for embodiment C1.

As previously, in a particular embodiment, the minimum detection threshold of the toxic algae of the genus Pseudo-nitzschia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment D1

The invention also concerns one of the methods for detecting active living cells of toxic algae as described above according to embodiments A1, B1 or C1, in a sample likely to contain in addition at least one toxic alga of the genus Prorocentrum, comprising in addition the addition of an optional complex formed between the ribosomal nucleic acid of a toxic alga of the genus Prorocentrum and a signal probe on a support containing a capture probe,

said capture probe and said signal probe forming a probe pair, the sequences of said probe pair being selected from x elements of one of the following sets:
(SEQ ID NO: 62, SEQ ID NO: 63 or SEQ ID NO: 64)
(SEQ ID NO: 65, SEQ ID NO: 66 or SEQ ID NO: 67)
(SEQ ID NO: 68, SEQ ID NO: 69 or SEQ ID NO: 70)
(SEQ ID NO: 71, SEQ ID NO: 72 or SEQ ID NO: 73)
   x being 2 or 3,
   or the sequences of the said probes having at least 92% identity with the abovementioned sequences SEQ ID NO: 62 to SEQ ID NO: 73
   hybridization indicating the presence of toxic algae of the genus Prorocentrum.

All the different types embodiment described for the detection method in embodiment A can be used for embodiment D1.

As previously, in a particular embodiment, the minimum detection threshold of the toxic algae of the genus Prorocentrum is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment E1

The invention also concerns one of the methods for detecting active living cells of toxic algae as described above according to embodiments A1, B1, C1, or D1, in a sample likely to contain in addition at least one toxic algae of the genus Chattonella, comprising in addition the addition of an optional complex formed between the ribosomal nucleic acid of a toxic algae of the genus Chattonella and a signal probe on a support containing a capture probe,

said capture probe and said signal probe forming a probe pair, the sequences of said probe pair being selected from x elements of one of the following sets:
(SEQ ID NO: 74, SEQ ID NO: 75 or SEQ ID NO: 76)
(SEQ ID NO: 77, SEQ ID NO: 78 or SEQ ID NO: 79)
(SEQ ID NO: 80, SEQ ID NO: 81 or SEQ ID NO: 82)
   x being 2 or 3,
   or the sequences of said probes having at least 92% identity with said sequences SEQ ID NO: 74 to SEQ ID NO: 82
   hybridization indicating the presence of toxic algae of the genus Chattonella.

All the different types embodiment described for the detection method in embodiment A can be used for embodiment E1.

As before, in a particular embodiment, the minimum detection threshold of the toxic algae of the genus Chatonella is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### F1 Embodiment

The invention also concerns one of the methods for detecting active living cells of toxic algae as described above according to embodiments A1, B1, C1, D1 or E1, in a sample likely to contain in addition at least one toxic alga of the genus Gymnodinium, comprising in addition the addition of an optional complex formed between the ribosomal nucleic acid of a toxic alga of the genus Gymnodinium and a signal probe on a support containing a capture probe, the capture probe and the signal probe forming a pair of probes, the sequences of said pair of probes being selected from x elements of one of the following sets:
(SEQ ID NO: 83, SEQ ID NO: 84 or SEQ ID NO: 85)
(SEQ ID NO: 86, SEQ ID NO: 87 or SEQ ID NO: 88)
(SEQ ID NO: 89, SEQ ID NO: 90 or SEQ ID NO: 91)
(SEQ ID NO: 92, SEQ ID NO: 93 or SEQ ID NO: 94)
   x being 2 or 3,
   or the sequences of said probes having at least 92% identity with the above-mentioned sequences SEQ ID NO: 83 to SEQ ID NO: 94
   hybridization indicating the presence of toxic algae of the genus Gymnodinium.

All the different embodiments described for the detection procedure in embodiment A can be applied for embodiment F1.

As previously, in a particular embodiment, the minimum detection threshold of the toxic algae of the genus Gymnodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment G1

The invention also concerns one of the methods for detecting active living cells of toxic algae as described above according to embodiments A1, B1, C1, D1, E1 or F1, in a sample likely to contain in addition at least one toxic algae of the genus Karenia, comprising in addition the addition of an optional complex formed between the ribosomal nucleic acid of a toxic algae of the genus Karenia and a signal probe on a support containing a capture probe,

the capture probe and the signal probe forming a pair of probes, the sequences of said pair of probes being selected from x elements of one of the following sets:
(SEQ ID NO: 95, SEQ ID NO: 96 or SEQ ID NO: 97)
(SEQ ID NO: 98, SEQ ID NO: 99 or SEQ ID NO: 100)
(SEQ ID NO: 101, SEQ ID NO: 102 or SEQ ID NO: 103)
(SEQ ID NO: 104, SEQ ID NO: 105 or SEQ ID NO: 106)
(SEQ ID NO: 107, SEQ ID NO: 108 or SEQ ID NO: 109)
(SEQ ID NO: 110, SEQ ID NO: 111 or SEQ ID NO: 112)
(SEQ ID NO: 113, SEQ ID NO: 114 or SEQ ID NO: 115)
(SEQ ID NO: 116, SEQ ID NO: 117 or SEQ ID NO: 118)
   x being 2 or 3,
   or the sequences of said probes having at least 92% identity with said sequences SEQ ID NO: 95 to SEQ ID NO: 118
   hybridization indicating the presence of toxic algae of the genus Karenia

All the different types embodiment described for the detection method in embodiment A can be used for embodiment G1.

As before, in a particular embodiment, the minimum detection threshold of the toxic algae of the genus Karenia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment H1

The invention also concerns one of the methods for detecting active living cells of toxic algae as described above according to embodiments A1, B1, C1, D1, E1, F1, or G1, in a sample likely to contain in addition at least one toxic algae of the genus Lingulodinium, comprising in addition the addition of an optional complex formed between the ribosomal nucleic acid of a toxic algae of the genus Lingulodinium and a signal probe on a support containing a capture probe,

the capture probe and the signal probe forming a pair of probes, the sequences of said pair of probes being selected from x elements of one of the following sets:
(SEQ ID NO: 119, SEQ ID NO: 120 or SEQ ID NO: 121)
(SEQ ID NO: 122, SEQ ID NO: 123 or SEQ ID NO: 124)
(SEQ ID NO: 125, SEQ ID NO: 126 or SEQ ID NO: 127)
   x being 2 or 3,
   or the sequences of said probes having at least 92% identity with said sequences SEQ ID NO: 119 to SEQ ID NO: 127
   hybridization indicating the presence of toxic algae of the genus Lingulodinium.

All the different types of embodiment described for the detection procedure in embodiment A can be used for Embodiment HE

As previously, in a particular embodiment, the minimum detection threshold of the toxic algae of the genus Lingulodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment I1

The invention also concerns one of the methods for detecting active living cells of toxic algae as described above according to embodiments A1, B1, C1, D1, E1, F1, G1 or H1, in a sample likely to contain in addition at least one toxic algae of the genus Heterosigma, comprising in addition the addition of an optional complex formed between the ribosomal nucleic acid of a toxic algae of the genus Heterosigma and a signal probe on a support containing a capture probe,

the capture probe and the signal probe forming a pair of probes, the sequences of said pair of probes being selected from x elements of one of the following sets:
(SEQ ID NO: 128 and SEQ ID NO: 129)
   x being 2 or 3,
   or the sequences of said probes having at least 92% identity with said sequences SEQ ID NO: 128 to SEQ ID NO: 129
   hybridization indicating the presence of toxic algae of the genus Heterosigma

All the different types embodiment described for the detection method in embodiment A can be used for embodiment I1.

As previously, in a particular embodiment, the minimum detection threshold of the toxic algae of the genus Heterosigma is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

As with the first aspect relating to use, all combinations of embodiments A1, B1, C1, D1, E1, F1, G1, H1 and/or I1 of this fourth aspect can be considered. In this way, all combinations of toxic algae B to 1128 described in the first aspect can be detected by the methods as described in this fourth aspect.

### Embodiment A2

Thus, according to another particular embodiment of this fourth aspect, the invention concerns a method of detecting active living cells of toxic algae in a sample likely to contain at least one toxic alga of the genus Alexandrium comprising the following steps:
a) addition of ribosomal nucleic acid from a toxic algae of the genus Alexandrium and a signal probe to a carrier containing a capture probe,
b) detection of the optional hybridization of a complex formed between said capture probe, said ribosomal nucleic acid and said signal probe, the hybridization taking place between the capture probe, the ribosomal nucleic acid and the signal probe, hybridization indicating the presence of toxic algae of the genus Alexandrium said capture probe and said signal probe forming a probe pair, the sequences of said probe pair being selected from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22),
   (SEQ ID NO: 23, SEQ ID NO: 24 or SEQ ID NO: 25),
   (SEQ ID NO: 26, SEQ ID NO: 27 or SEQ ID NO: 28)
   x being 2 or 3,
   or the sequences of said probes having at least 92% identity with said sequences SEQ ID NO: 1 to SEQ ID NO: 28.

All the different embodiments described for the detection method in embodiment A can be used for embodiment A2.

As before, in a particular embodiment, the minimum detection threshold of the toxic algae of the genus Alexandrium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment B2

The invention also concerns a method for detecting active living cells of toxic algae as described above according to embodiment A2, in a sample likely to contain in addition at least one toxic alga of the genus Dinophysis, comprising, in addition, the addition of the ribosomal nucleic acid of a toxic alga of the genus Dinophysis and a signal probe to a support containing a capture probe,

said capture probe and said signal probe forming a probe pair, the sequences of said probe pair being selected from x elements of one of the following sets:
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44 or SEQ ID NO: 45), or
(SEQ ID NO: 46, SEQ ID NO: 47 or SEQ ID NO: 48)
   x being 2 or 3,
   or the sequences of said probes having at least 92% identity with said sequences SEQ ID NO: 29 to SEQ ID NO: 48
   hybridization indicating the presence of toxic algae of the genus Dinophysis.

All of the different types embodiment described for the detection method in embodiment A can be used for embodiment B2.

As previously, in a particular embodiment, the minimum detection threshold of the toxic algae of the genus Dinophysis is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment C2

The invention also concerns one of the methods for detecting active living cells of toxic algae as described above according to embodiments A2 or B2, in a sample likely to contain in addition at least one toxic alga of the genus Pseudo-nitzschia, comprising in addition the addition of the ribosomal nucleic acid of a toxic alga of the genus Pseudo-nitzschia and a signal probe to a support containing a capture probe,

said capture probe and said signal probe forming a probe pair, the sequences of said probe pair being selected from x elements of one of the following sets:
(SEQ ID NO: 49 and SEQ ID NO: 50)
(SEQ ID NO: 51 and SEQ ID NO: 52)
(SEQ ID NO: 53, SEQ ID NO: 54 or SEQ ID NO: 55)
(SEQ ID NO: 56, SEQ ID NO: 57 or SEQ ID NO: 58), or
(SEQ ID NO: 59, SEQ ID NO: 60 or SEQ ID NO: 61)
   x being 2 or 3,
   or the sequences of said probes having at least 92% identity with said sequences SEQ ID NO: 49 to SEQ ID NO: 61
   hybridization indicating the presence of toxic algae of the genus Pseudo-nitzschia.

All of the different embodiments described for the detection method in embodiment A can be used for embodiment C2.

As previously, in a particular embodiment, the minimum detection threshold of the toxic algae of the genus Pseudo-nitzschia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment D2

The invention also concerns one of the methods for detecting active living cells of toxic algae as described above according to embodiments A2, B2 or C2, in a sample likely to contain in addition at least one toxic alga of the genus Prorocentrum, comprising in addition the addition of the ribosomal nucleic acid of a toxic alga of the genus Prorocentrum and a signal probe to a support containing a capture probe,

said capture probe and said signal probe forming a probe pair, the sequences of said probe pair being selected from x elements of one of the following sets:
(SEQ ID NO: 62, SEQ ID NO: 63 or SEQ ID NO: 64)
(SEQ ID NO: 65, SEQ ID NO: 66 or SEQ ID NO: 67)
(SEQ ID NO: 68, SEQ ID NO: 69 or SEQ ID NO: 70)
(SEQ ID NO: 71, SEQ ID NO: 72 or SEQ ID NO: 73)
   x being 2 or 3,
   or the sequences of the said probes having at least 92% identity with the abovementioned sequences SEQ ID NO: 62 to SEQ ID NO: 73
   hybridization indicating the presence of toxic algae of the genus Prorocentrum.

All the different types embodiment described for the detection method in embodiment A can be used for embodiment D2.

As before, in a particular embodiment, the minimum detection threshold of the toxic algae of the genus Prorocentrum is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment E2

The invention also relates to one of the methods for detecting active living cells of toxic algae as described above according to embodiments A2, B2, C2 or D2, in a sample likely to contain in addition at least one toxic algae of the genus Chattonella, comprising in addition the addition of the ribosomal nucleic acid of a toxic algae of the genus Chattonella and a signal probe to a support containing a capture probe,

said capture probe and said signal probe forming a probe pair, the sequences of said probe pair being selected from x elements of one of the following sets:
(SEQ ID NO: 74, SEQ ID NO: 75 or SEQ ID NO: 76)
(SEQ ID NO: 77, SEQ ID NO: 78 or SEQ ID NO: 79)
(SEQ ID NO: 80, SEQ ID NO: 81 or SEQ ID NO: 82)
   x being 2 or 3,
   or the sequences of said probes having at least 92% identity with said sequences SEQ ID NO: 74 to SEQ ID NO: 82
   hybridization indicating the presence of toxic algae of the genus Chattonella.

All the different types embodiment described for the detection method in embodiment A can be used for embodiment E2.

As previously, in a particular embodiment, the minimum detection threshold of the toxic algae of the genus Chattonella is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment F2

The invention also relates to one of the methods for detecting active living cells of toxic algae as described above according to embodiments A2, B2, C2, D2 or E2, in a sample likely to contain in addition at least one toxic algae of the genus Gymnodinium, comprising in addition the addition of the ribosomal nucleic acid of a toxic algae of the genus Gymnodinium and a signal probe to a support containing a capture probe,

the capture probe and the signal probe forming a pair of probes, the sequences of said pair of probes being selected from x elements of one of the following sets:
(SEQ ID NO: 83, SEQ ID NO: 84 or SEQ ID NO: 85)
(SEQ ID NO: 86, SEQ ID NO: 87 or SEQ ID NO: 88)
(SEQ ID NO: 89, SEQ ID NO: 90 or SEQ ID NO: 91)
(SEQ ID NO: 92, SEQ ID NO: 93 or SEQ ID NO: 94)
   x being 2 or 3,
   or the sequences of said probes having at least 92% identity with the above-mentioned sequences SEQ ID NO: 83 to SEQ ID NO: 94
   hybridization indicating the presence of toxic algae of the genus Gymnodinium.

All the different types embodiment described for the detection procedure in embodiment A can be applied for embodiment F2.

As previously, in a particular embodiment, the minimum detection threshold of the toxic algae of the genus Gymnodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment G2

The invention also relates to one of the methods for detecting active living cells of toxic algae as described above according to embodiments A2, B2, C2, D2, E2 or F2, in a sample likely to contain in addition at least one toxic algae of the genus Karenia, comprising in addition the addition of the ribosomal nucleic acid of a toxic algae of the genus Karenia and a signal probe to a support containing a capture probe,

the capture probe and the signal probe forming a pair of probes, the sequences of said pair of probes being selected from x elements of one of the following sets:
(SEQ ID NO: 95, SEQ ID NO: 96 or SEQ ID NO: 97)
(SEQ ID NO: 98, SEQ ID NO: 99 or SEQ ID NO: 100)
(SEQ ID NO: 101, SEQ ID NO: 102 or SEQ ID NO: 103)
(SEQ ID NO: 104, SEQ ID NO: 105 or SEQ ID NO: 106)
(SEQ ID NO: 107, SEQ ID NO: 108 or SEQ ID NO: 109)
(SEQ ID NO: 110, SEQ ID NO: 111 or SEQ ID NO: 112)
(SEQ ID NO: 113, SEQ ID NO: 114 or SEQ ID NO: 115)
(SEQ ID NO: 116, SEQ ID NO: 117 or SEQ ID NO: 118)
   x being 2 or 3,
   or the sequences of said probes having at least 92% identity with said sequences SEQ ID NO: 95 to SEQ ID NO: 118
   hybridization indicating the presence of toxic algae of the genus Karenia.

All the different types embodiment described for the detection method in embodiment A can be used for embodiment G2.

As before, in a particular embodiment, the minimum detection threshold of the toxic algae of the genus Karenia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment H2

The invention also concerns one of the methods for detecting active living cells of toxic algae as described above according to embodiments A2, B2, C2, D2, E2, F2 or G2, in a sample which may additionally contain at least one toxic algae of the genus Lingulodinium, comprising in addition the addition of the ribosomal nucleic acid of a toxic algae of the genus Lingulodinium and a signal probe to a support containing a capture probe,

the capture probe and the signal probe forming a pair of probes, the sequences of said pair of probes being selected from x elements of one of the following sets:
(SEQ ID NO: 119, SEQ ID NO: 120 or SEQ ID NO: 121)
(SEQ ID NO: 122, SEQ ID NO: 123 or SEQ ID NO: 124)
(SEQ ID NO: 125, SEQ ID NO: 126 or SEQ ID NO: 127)
   x being 2 or 3,
   or the sequences of said probes having at least 92% identity with said sequences SEQ ID NO: 119 to SEQ ID NO: 127
   hybridization indicating the presence of toxic algae of the genus Lingulodinium.

All the different types of embodiments described for the detection method in embodiment A can be used for embodiment H2.

As previously, in a particular embodiment, the minimum detection threshold of the toxic algae of the genus Lingulodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment I2

The invention also concerns one of the methods for detecting active living cells of toxic algae as described above according to embodiments A2, B2, C2, D2, E2, F2, G2 or H2 in a sample which may additionally contain at least one toxic alga of the genus Heterosigma, comprising in addition the addition of the ribosomal nucleic acid of a toxic alga of the genus Heterosigma and a signal probe to a support containing a capture probe,

the capture probe and the signal probe forming a pair of probes, the sequences of said pair of probes being selected from x elements of one of the following sets:
(SEQ ID NO: 128 and SEQ ID NO: 129)
   x being 2 or 3,
   or the sequences of said probes having at least 92% identity with said sequences SEQ ID NO: 128 to SEQ ID NO: 129
   hybridization indicating the presence of toxic algae of the genus Heterosigma.

All the different types embodiment described for the detection method in embodiment A can be used for embodiment 12.

As before, in a particular embodiment, the minimum detection threshold of the toxic algae of the genus Heterosigma is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

As with the first aspect relating to use, all combinations of embodiments A2, B2, C2, D2, E2, F2, G2, H2 and/or I2 of this fourth aspect can be considered. In this way, all the combinations of toxic algae B to 1128 described in the first aspect can be detected by the methods as described in this fourth aspect.

### Embodiment A3

Thus, according to another particular embodiment of this fourth aspect, the invention concerns a method of detecting active living cells of toxic algae in a sample likely to contain at least one toxic alga of the genus Alexandrium comprising the following steps:
a) addition of a complex formed between:
   the ribosomal nucleic acid of a toxic algae of the genus Alexandrium
   a signal probe
   and a capture probe
   on a support,
b) detection of the optional hybridization of the above-mentioned complex, the hybridization taking place between the capture probe, the ribosomal nucleic acid and the signal probe,
   hybridization indicating the presence of toxic algae of the genus Alexandrium said capture probe and said signal probe forming a probe pair, the sequences of said probe pair being selected from x elements of one of the following sets:
      (SEQ ID NO: 1, SEQ ID NO: 2 or SEQ ID NO: 3)
      (SEQ ID NO: 4 and SEQ ID NO: 5)
      (SEQ ID NO: 6 and SEQ ID NO: 7)
      (SEQ ID NO: 8, SEQ ID NO: 9 or SEQ ID NO: 10)
      (SEQ ID NO: 11, SEQ ID NO: 12 or SEQ ID NO: 13)
      (SEQ ID NO: 14, SEQ ID NO: 15 or SEQ ID NO: 16)
      (SEQ ID NO: 17, SEQ ID NO: 18 or SEQ ID NO: 19)
      (SEQ ID NO: 20, SEQ ID NO: 21 or SEQ ID NO: 22),
      (SEQ ID NO: 23, SEQ ID NO: 24 or SEQ ID NO: 25),
      (SEQ ID NO: 26, SEQ ID NO: 27 or SEQ ID NO: 28)
   x being 2 or 3,
   or the sequences of said probes having at least 92% identity with said sequences SEQ ID NO: 1 to SEQ ID NO: 28.

All the different embodiments described for the detection method in embodiment A can be used for embodiment A3.

As before, in a particular embodiment, the minimum detection threshold of the toxic algae of the genus Alexandrium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment B3

The invention also relates to a method for detecting active living cells of toxic algae as described above according to method A3, in a sample likely to contain in addition at least one toxic algae of the genus Dinophysis, comprising, in addition, the addition of a complex formed between:
the ribosomal nucleic acid of a toxic algae of the genus Dinophysis
a signal probe
and a capture probe
   on a support,
   said capture probe and said signal probe forming a probe pair, the sequences of said probe pair being selected from x elements of one of the following sets:
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44 or SEQ ID NO: 45), or
(SEQ ID NO: 46, SEQ ID NO: 47 or SEQ ID NO: 48)
   x being 2 or 3,
   or the sequences of said probes having at least 92% identity with said sequences SEQ ID NO: 29 to SEQ ID NO: 48
   hybridization indicating the presence of toxic algae of the genus Dinophysis.

All the different embodiments described for the detection method in embodiment A can be used in embodiment B3.

As previously, in a particular embodiment, the minimum detection threshold of the toxic algae of the genus Dinophysis is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment C3

The invention also relates to one of the methods for detecting active living cells of toxic algae as described above according to embodiments A3 or B3, in a sample likely to contain in addition at least one toxic algae of the genus Pseudo-nitzschia, comprising in addition the addition of a complex formed between:
the ribosomal nucleic acid of a toxic algae of the genus Pseudo-nitzschia
a signal probe
and a capture probe
   on a support,
   said capture probe and said signal probe forming a probe pair, the sequences of said probe pair being selected from x elements of one of the following sets:
(SEQ ID NO: 49 and SEQ ID NO: 50)
(SEQ ID NO: 51 and SEQ ID NO: 52)
(SEQ ID NO: 53, SEQ ID NO: 54 or SEQ ID NO: 55)
(SEQ ID NO: 56, SEQ ID NO: 57 or SEQ ID NO: 58), or
(SEQ ID NO: 59, SEQ ID NO: 60 or SEQ ID NO: 61)
   x being 2 or 3,
   or the sequences of said probes having at least 92% identity with said sequences SEQ ID NO: 49 to SEQ ID NO: 61
   hybridization indicating the presence of toxic algae of the genus Pseudo-nitzschia.

All the different embodiments described for the detection method in embodiment A can be used for embodiment C3.

As previously, in a particular embodiment, the minimum detection threshold of the toxic algae of the genus Pseudo-nitzschia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment D3

The invention also concerns one of the methods for detecting active living cells of toxic algae as described above according to embodiments A3, B3 or C3, in a sample likely to contain in addition at least one toxic algae of the genus Prorocentrum, comprising in addition the addition of a complex formed between:
the ribosomal nucleic acid of a toxic algae of the genus Prorocentrum
a signal probe
and a capture probe
   on a support,
   said capture probe and said signal probe forming a probe pair, the sequences of said probe pair being selected from x elements of one of the following sets:
(SEQ ID NO: 62, SEQ ID NO: 63 or SEQ ID NO: 64)
(SEQ ID NO: 65, SEQ ID NO: 66 or SEQ ID NO: 67)
(SEQ ID NO: 68, SEQ ID NO: 69 or SEQ ID NO: 70)
(SEQ ID NO: 71, SEQ ID NO: 72 or SEQ ID NO: 73)
   x being 2 or 3,
   or the sequences of the said probes having at least 92% identity with the abovementioned sequences SEQ ID NO: 62 to SEQ ID NO: 73
   hybridization indicating the presence of toxic algae of the genus Prorocentrum.

All the different types embodiment described for the detection method in embodiment A can be used for embodiment D3.

As previously, in a particular embodiment, the minimum detection threshold of the toxic algae of the genus Prorocentrum is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment E3

The invention also concerns one of the methods for detecting active living cells of toxic algae as described above according to embodiments A3, B3, C3 or D3, in a sample likely to contain in addition at least one toxic algae of the genus Chattonella, comprising in addition the addition of a complex formed between:
the ribosomal nucleic acid of a toxic algae of the genus Chattonella
a signal probe
and a capture probe
   on a support,
   said capture probe and said signal probe forming a probe pair, the sequences of said probe pair being selected from x elements of one of the following sets:
(SEQ ID NO: 74, SEQ ID NO: 75 or SEQ ID NO: 76)
(SEQ ID NO: 77, SEQ ID NO: 78 or SEQ ID NO: 79)
(SEQ ID NO: 80, SEQ ID NO: 81 or SEQ ID NO: 82)
   x being 2 or 3,
   or the sequences of said probes having at least 92% identity with said sequences SEQ ID NO: 74 to SEQ ID NO: 82
   hybridization indicating the presence of toxic algae of the genus Chattonella.

All the different types embodiment described for the detection method in embodiment A can be used for embodiment E3.

As before, in a particular embodiment, the minimum detection threshold of the toxic algae of the genus Chatonella is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment F3

The invention also concerns one of the methods for detecting active living cells of toxic algae as described above according to embodiments A3, B3, C3, D3 or E3, in a sample likely to contain in addition at least one toxic algae of the genus Gymnodinium, comprising in addition the addition of a complex formed between:
the ribosomal nucleic acid of a toxic algae of the genus Gymnodinium
a signal probe
and a capture probe
   on a support,
   the capture probe and the signal probe forming a pair of probes, the sequences of said pair of probes being selected from x elements of one of the following sets:
(SEQ ID NO: 83, SEQ ID NO: 84 or SEQ ID NO: 85)
(SEQ ID NO: 86, SEQ ID NO: 87 or SEQ ID NO: 88)
(SEQ ID NO: 89, SEQ ID NO: 90 or SEQ ID NO: 91)
(SEQ ID NO: 92, SEQ ID NO: 93 or SEQ ID NO: 94)
   x being 2 or 3,
   or the sequences of said probes having at least 92% identity with the above-mentioned sequences SEQ ID NO: 83 to SEQ ID NO: 94
   hybridization indicating the presence of toxic algae of the genus Gymnodinium.

All the different types embodiment described for the detection method in embodiment A can be used for embodiment F3.

As previously, in a particular embodiment, the minimum detection threshold of the toxic algae of the genus Gymnodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment G3

The invention also relates to one of the methods for detecting active living cells of toxic algae as described above according to embodiments A3, B3, C3, D3, E3 or F3, in a sample likely to contain in addition at least one toxic algae of the genus Karenia, comprising in addition the addition of a complex formed between:
the ribosomal nucleic acid of a toxic algae of the genus Karenia
a signal probe
and a capture probe
   on a support,
   the capture probe and the signal probe forming a pair of probes, the sequences of said pair of probes being selected from x elements of one of the following sets:
(SEQ ID NO: 95, SEQ ID NO: 96 or SEQ ID NO: 97)
(SEQ ID NO: 98, SEQ ID NO: 99 or SEQ ID NO: 100)
(SEQ ID NO: 101, SEQ ID NO: 102 or SEQ ID NO: 103)
(SEQ ID NO: 104, SEQ ID NO: 105 or SEQ ID NO: 106)
(SEQ ID NO: 107, SEQ ID NO: 108 or SEQ ID NO: 109)
(SEQ ID NO: 110, SEQ ID NO: 111 or SEQ ID NO: 112)
(SEQ ID NO: 113, SEQ ID NO: 114 or SEQ ID NO: 115)
(SEQ ID NO: 116, SEQ ID NO: 117 or SEQ ID NO: 118)
   x being 2 or 3,
   or the sequences of said probes having at least 92% identity with said sequences SEQ ID NO: 95 to SEQ ID NO: 118
   hybridization indicating the presence of toxic algae of the genus Karenia.

All the different embodiments described for the detection method in embodiment A can be used for embodiment G3.

As before, in a particular embodiment, the minimum detection threshold of the toxic algae of the genus Karenia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment H3

The invention also concerns one of the methods for detecting active living cells of toxic algae as described above according to embodiments A3, B3, C3, D3, E3, F3 or G3, in a sample which may also contain at least one toxic algae of the genus Lingulodinium, comprising in addition the addition of a complex formed between:
the ribosomal nucleic acid of a toxic algae of the genus Lingulodinium
a signal probe
and a capture probe
   on a support,
   the capture probe and the signal probe forming a pair of probes, the sequences of said pair of probes being selected from x elements of one of the following sets:
(SEQ ID NO: 119, SEQ ID NO: 120 or SEQ ID NO: 121)
(SEQ ID NO: 122, SEQ ID NO: 123 or SEQ ID NO: 124)
(SEQ ID NO: 125, SEQ ID NO: 126 or SEQ ID NO: 127)
   x being 2 or 3,
   or the sequences of said probes having at least 92% identity with said sequences SEQ ID NO: 119 to SEQ ID NO: 127
   hybridization indicating the presence of toxic algae of the genus Lingulodinium.

All the different embodiments described for the detection method in embodiment A can be used for embodiment H3.

As previously, in a particular embodiment, the minimum detection threshold of the toxic algae of the genus Lingulodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment I3

The invention also concerns one of the methods for detecting active living cells of toxic algae as described above according to embodiments A3, B3, C3, D3, E3, F3, G3 or H3, in a sample likely to contain in addition at least one toxic algae of the genus Heterosigma, comprising in addition the addition of a complex formed between:
the ribosomal nucleic acid of a toxic algae of the genus Heterosigma
a signal probe
and a capture probe
   on a support,
   the capture probe and the signal probe forming a pair of probes, the sequences of said pair of probes being selected from x elements of one of the following sets:
(SEQ ID NO: 128 and SEQ ID NO: 129)
   x being 2 or 3,
   or the sequences of said probes having at least 92% identity with said sequences SEQ ID NO: 128 to SEQ ID NO: 129
   hybridization indicating the presence of toxic algae of the genus Heterosigma.

All the different types embodiment described for the detection method in embodiment A can be used for embodiment 13.

As before, in a particular embodiment, the minimum detection threshold of the toxic algae of the genus Heterosigma is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

As with the first aspect relating to use, all combinations of embodiments A3, B3, C3, D3, E3, F3, G3, H3 and/or I3 of this fourth aspect can be considered. In this way, all combinations of toxic algae B to 1128 described in the first aspect can be detected by the methods as described in this fourth aspect.

### Embodiment A4

According to another particular embodiment of this fourth aspect, the invention concerns a method of detecting active living cells of toxic algae in a sample likely to contain at least one toxic alga of the genus Alexandrium comprising the following steps:
a) addition of a signal probe and an optional complex formed between the ribosomal nucleic acid of a toxic algae of the genus Alexandrium and a probe captured on a support,
b) detection of the optional hybridization of said complex with said signal probe, the hybridization taking place between the signal probe and the ribosomal nucleic acid of said complex, hybridization indicating the presence of toxic algae of the genus Alexandrium said capture probe and said signal probe forming a probe pair, the sequences of said probe pair being selected from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22),
   (SEQ ID NO: 23, SEQ ID NO: 24 or SEQ ID NO: 25),
   (SEQ ID NO: 26, SEQ ID NO: 27 or SEQ ID NO: 28)
   x being 2 or 3,
   or the sequences of said probes having at least 92% identity with said sequences SEQ ID NO: 1 to SEQ ID NO: 28.

All the different embodiments described for the detection method in embodiment A can be applied for embodiment A4.

As before, in a particular embodiment, the minimum detection threshold of the toxic algae of the genus Alexandrium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment B4

The invention also concerns a method for detecting active living cells of toxic algae as described above according to embodiment A4, in a sample likely to contain in addition at least one toxic algae of the genus Dinophysis, comprising, in addition, the addition of a signal probe and an optional complex formed between the ribosomal nucleic acid of a toxic algae of the genus Dinophysis and a probe captured on a support,

said capture probe and said signal probe forming a probe pair, the sequences of said probe pair being selected from x elements of one of the following sets:
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44 or SEQ ID NO: 45), or
(SEQ ID NO: 46, SEQ ID NO: 47 or SEQ ID NO: 48)
   x being 2 or 3,
   or the sequences of said probes having at least 92% identity with said sequences SEQ ID NO: 29 to SEQ ID NO: 48
   hybridization indicating the presence of toxic algae of the genus Dinophysis.

All the different embodiments described for the detection method in embodiment A can be used for embodiment B4.

As previously, in a particular embodiment, the minimum detection threshold of the toxic algae of the genus Dinophysis is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment C4

The invention also relates to one of the methods for detecting active living cells of toxic algae as described above according to embodiments A4 or B4, in a sample likely to contain in addition at least one toxic algae of the genus Pseudo-nitzschia, comprising in addition the addition of a signal probe and an optional complex formed between the ribosomal nucleic acid of a toxic algae of the genus Pseudo-nitzschia and a probe captured on a support,

said capture probe and said signal probe forming a probe pair, the sequences of said probe pair being selected from x elements of one of the following sets:
(SEQ ID NO: 49 and SEQ ID NO: 50)
(SEQ ID NO: 51 and SEQ ID NO: 52)
(SEQ ID NO: 53, SEQ ID NO: 54 or SEQ ID NO: 55)
(SEQ ID NO: 56, SEQ ID NO: 57 or SEQ ID NO: 58), or
(SEQ ID NO: 59, SEQ ID NO: 60 or SEQ ID NO: 61)
   x being 2 or 3,
   or the sequences of said probes having at least 92% identity with said sequences SEQ ID NO: 49 to SEQ ID NO: 61
   hybridization indicating the presence of toxic algae of the genus Pseudo-nitzschia.

All the different types embodiment described for the detection procedure in embodiment A can be used for embodiment C4.

As previously, in a particular embodiment, the minimum detection threshold of the toxic algae of the genus Pseudo-nitzschia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment D4

The invention also concerns one of the methods for detecting active living cells of toxic algae as described above according to embodiments A4, B4 or C4, in a sample likely to contain in addition at least one toxic algae of the genus Prorocentrum, comprising in addition the addition of a signal probe and an optional complex formed between the ribosomal nucleic acid of a toxic algae of the genus Prorocentrum and a probe captured on a support,

said capture probe and said signal probe forming a probe pair, the sequences of said probe pair being selected from x elements of one of the following sets:
(SEQ ID NO: 62, SEQ ID NO: 63 or SEQ ID NO: 64)
(SEQ ID NO: 65, SEQ ID NO: 66 or SEQ ID NO: 67)
(SEQ ID NO: 68, SEQ ID NO: 69 or SEQ ID NO: 70)
(SEQ ID NO: 71, SEQ ID NO: 72 or SEQ ID NO: 73)
   x being 2 or 3,
   or the sequences of the said probes having at least 92% identity with the abovementioned sequences SEQ ID NO: 62 to SEQ ID NO: 73
   hybridization indicating the presence of toxic algae of the genus Prorocentrum.

All the different types embodiment described for the detection method in embodiment A can be used for embodiment D4.

As before, in a particular embodiment, the minimum detection threshold of the toxic algae of the genus Prorocentrum is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment E4

The invention also concerns one of the methods for detecting active living cells of toxic algae as described above according to embodiments A4, B4, C4 or D4, in a sample likely to contain in addition at least one toxic algae of the genus Chattonella, comprising in addition the addition of a signal probe and an optional complex formed between the ribosomal nucleic acid of a toxic algae of the genus Chattonella and a probe captured on a support,

said capture probe and said signal probe forming a probe pair, the sequences of said probe pair being selected from x elements of one of the following sets:
(SEQ ID NO: 74, SEQ ID NO: 75 or SEQ ID NO: 76)
(SEQ ID NO: 77, SEQ ID NO: 78 or SEQ ID NO: 79)
(SEQ ID NO: 80, SEQ ID NO: 81 or SEQ ID NO: 82)
   x being 2 or 3,
   or the sequences of said probes having at least 92% identity with said sequences SEQ ID NO: 74 to SEQ ID NO: 82
   hybridization indicating the presence of toxic algae of the genus Chattonella.

All the different types embodiment described for the detection method in embodiment A can be used for embodiment E4.

As previously, in a particular embodiment, the minimum detection threshold of the toxic algae of the genus Chattonella is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment F4

The invention also concerns one of the methods for detecting active living cells of toxic algae as described above according to embodiments A4, B4, C4, D4 or E4, in a sample likely to contain in addition at least one toxic algae of the genus Gymnodinium, comprising in addition the addition of a signal probe and an optional complex formed between the ribosomal nucleic acid of a toxic algae of the genus Gymnodinium and a probe captured on a support,

the capture probe and the signal probe forming a pair of probes, the sequences of said pair of probes being selected from x elements of one of the following sets:
(SEQ ID NO: 83, SEQ ID NO: 84 or SEQ ID NO: 85)
(SEQ ID NO: 86, SEQ ID NO: 87 or SEQ ID NO: 88)
(SEQ ID NO: 89, SEQ ID NO: 90 or SEQ ID NO: 91)
(SEQ ID NO: 92, SEQ ID NO: 93 or SEQ ID NO: 94)
   x being 2 or 3,
   or the sequences of said probes having at least 92% identity with the above-mentioned sequences SEQ ID NO: 83 to SEQ ID NO: 94
   hybridization indicating the presence of toxic algae of the genus Gymnodinium.

All the different types embodiment described for the detection procedure in embodiment A can be applied for embodiment F4.

As previously, in a particular embodiment, the minimum detection threshold of the toxic algae of the genus Gymnodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment G4

The invention also concerns one of the methods for detecting active living cells of toxic algae as described above according to embodiments A4, B4, C4, D4, E4 or F4, in a sample likely to contain in addition at least one toxic algae of the genus Karenia, comprising in addition the addition of a signal probe and an optional complex formed between the ribosomal nucleic acid of a toxic algae of the genus Karenia and a probe captured on a support,

the capture probe and the signal probe forming a pair of probes, the sequences of said pair of probes being selected from x elements of one of the following sets:
(SEQ ID NO: 95, SEQ ID NO: 96 or SEQ ID NO: 97)
(SEQ ID NO: 98, SEQ ID NO: 99 or SEQ ID NO: 100)
(SEQ ID NO: 101, SEQ ID NO: 102 or SEQ ID NO: 103)
(SEQ ID NO: 104, SEQ ID NO: 105 or SEQ ID NO: 106)
(SEQ ID NO: 107, SEQ ID NO: 108 or SEQ ID NO: 109)
(SEQ ID NO: 110, SEQ ID NO: 111 or SEQ ID NO: 112)
(SEQ ID NO: 113, SEQ ID NO: 114 or SEQ ID NO: 115)
(SEQ ID NO: 116, SEQ ID NO: 117 or SEQ ID NO: 118)
   x being 2 or 3,
   or the sequences of said probes having at least 92% identity with said sequences SEQ ID NO: 95 to SEQ ID NO: 118
   hybridization indicating the presence of toxic algae of the genus Karenia.

All the different types embodiment described for the detection method in embodiment A can be used for embodiment G4.

As before, in a particular embodiment, the minimum detection threshold of the toxic algae of the genus Karenia is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment H4

The invention also concerns one of the methods for detecting active living cells of toxic algae as described above according to embodiments A4, B4, C4, D4, E4, F4 or G4, in a sample which may additionally contain at least one toxic algae of the genus Lingulodinium, comprising in addition the addition of a signal probe and an optional complex formed between the ribosomal nucleic acid of a toxic algae of the genus Lingulodinium and a probe captured

on a support,
the capture probe and the signal probe forming a pair of probes, the sequences of said pair of probes being selected from x elements of one of the following sets:
(SEQ ID NO: 119, SEQ ID NO: 120 or SEQ ID NO: 121)
(SEQ ID NO: 122, SEQ ID NO: 123 or SEQ ID NO: 124)
(SEQ ID NO: 125, SEQ ID NO: 126 or SEQ ID NO: 127)
   x being 2 or 3,
   or the sequences of said probes having at least 92% identity with said sequences SEQ ID NO: 119 to SEQ ID NO: 127
   hybridization indicating the presence of toxic algae of the genus Lingulodinium.

All the different embodiments described for the detection method in embodiment A can be used for embodiment H4.

As previously, in a particular embodiment, the minimum detection threshold of the toxic algae of the genus Lingulodinium is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

### Embodiment I4

The invention also concerns one of the methods for detecting active living cells of toxic algae as described above according to embodiments A4, B4, C4, D4, E4, F4, G4 or H4, in a sample likely to contain in addition at least one toxic algae of the genus Heterosigma, comprising in addition the addition of a signal probe and an optional complex formed between the ribosomal nucleic acid of a toxic algae of the genus Heterosigma and a probe captured on a support,

the capture probe and the signal probe forming a pair of probes, the sequences of said pair of probes being selected from x elements of one of the following sets:
(SEQ ID NO: 128 and SEQ ID NO: 129)
   x being 2 or 3,
   or the sequences of said probes having at least 92% identity with said sequences SEQ ID NO: 128 to SEQ ID NO: 129
   hybridization indicating the presence of toxic algae of the genus Heterosigma.

All the different types embodiment described for the detection method in embodiment A can be used for embodiment 14.

As before, in a particular embodiment, the minimum detection threshold of the toxic algae of the genus Heterosigma is
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample.

In the same way, in a particular embodiment, the duration of the implementation of the said detection method is less than one hour.

As with the first aspect relating to use, any combination of the embodiments A4, B4, C4, D4, E4, F4, G4, H4 and/or I4 of this fourth aspect can be considered. In this way, all combinations of toxic algae B to 1128 described in the first aspect can be detected by the methods as described in this fourth aspect.

According to one embodiment, and in all embodiments of this fourth aspect, positive control can be used. The positive control can, for example, be a synthetic nucleic acid complementary to the capture probe and the signal probe. The positive control can also be used as a standard.

According to one embodiment, and in all embodiments of this fourth aspect, a negative control can be used. For example, the negative control can be a synthetic nucleic acid that is non-complementary to the capture probe and the signal probe.

According to one embodiment, and in all embodiments of this fourth aspect, the simultaneous detection of several algae is optional. In this case, the simultaneous detection is carried out on the same medium, but separately. For example, in the case where the support is a microplate, the detection of each algae to be detected is done in separate wells of the microplate.

A fifth aspect of the invention concerns kits for the detection of active living cells of toxic algae.

### Embodiment A

Thus, according to this fifth aspect, the invention concerns a kit for the detection of active living cells of toxic algae of toxic algae of the genus Alexandrium, said kit containing:
a) at least one pair of probes specific to toxic algae of the genus Alexandrium, the sequences of said probes being selected from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22)
   (SEQ ID NO: 23, SEQ ID NO: 24 or SEQ ID NO: 25)
   (SEQ ID NO: 26, SEQ ID NO: 27 or SEQ ID NO: 28)
   x being 2 or 3,
   or the sequences of said probes having at least 92% identity with said sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28,
   one probe of said pair being a capture probe linked to at least one attachment molecule positioned at 3′ or 5′ of its sequence and the other probe of said pair being a signal probe linked to at least one marker molecule positioned at 3′ or 5′ of its sequence said capture probe and said signal probe being capable of hybridizing with the ribosomal nucleic acid of a toxic algae of the genus Alexandrium,
b) optionally a hybridization solution
c) optionally a washing solution, and
d) optionally a revelation solution.

### Embodiment B

The invention also concerns a kit as described above according to embodiment A, for the detection of active living cells of toxic algae of toxic algae of the genus Alexandrium and/or Dinophysis, said kit containing in addition:
a) at least one pair of probes specific to toxic algae of the genus Dinophysis, the sequences of said probes being selected from x elements of one of the following sets:
   (SEQ ID NO: 29, SEQ ID NO: 30 or SEQ ID NO: 31)
   (SEQ ID NO: 32 and SEQ ID NO: 33)
   (SEQ ID NO: 34 and SEQ ID NO: 35)
   (SEQ ID NO: 36 and SEQ ID NO: 37)
   (SEQ ID NO: 38 and SEQ ID NO: 39)
   (SEQ ID NO: 40, SEQ ID NO: 41 or SEQ ID NO: 42)
   (SEQ ID NO: 3, SEQ ID NO: 44 or SEQ ID NO: 45), or
   (SEQ ID NO: 46, SEQ ID NO: 47 or SEQ ID NO: 48)
   x being 2 or 3,
   or the sequences of said probes having at least 92% identity with the abovementioned sequences SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48
   one probe of said pair being a capture probe linked to at least one attachment molecule positioned at 3′ or 5′ of its sequence and the other probe of said pair being a signal probe linked to at least one marker molecule positioned at 3′ or 5′ of its sequence said capture probe and said signal probe being capable of hybridizing with the ribosomal nucleic acid of a toxic algae of the genus Dinophysis.

### Embodiment C

The invention also relates to a kit as described above according to embodiment A or according to embodiment B, for the detection of active living cells of toxic algae of toxic algae of the genus Alexandrium and/or Pseudo-nitzschia, said kit additionally containing:
a) at least one pair of probes specific to toxic algae of the genus Pseudo-nitzschia, the sequences of said probes being selected from x elements of one of the following sets:
   (SEQ ID NO: 49 and SEQ ID NO: 50)
   (SEQ ID NO: 51 and SEQ ID NO: 52)
   (SEQ ID NO: 53, SEQ ID NO: 54 or SEQ ID NO: 55)
   (SEQ ID NO: 56, SEQ ID NO: 57, SEQ ID NO: 58)
   (SEQ ID NO: 59, SEQ ID NO: 60, SEQ ID NO: 61)
   x being 2 or 3,
   or the sequences of said probes having at least 92% identity with the abovementioned sequences SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic algae of the genus Pseudo-nitzschia.

### Embodiment D

The invention also concerns a kit as described above according to embodiments A, B or C, for the detection of active living cells of toxic algae of toxic algae of the genus Alexandrium and/or Prorocentrum, said kit containing in addition:
a) at least one pair of probes specific to toxic algae of the genus Prorocentrum, the sequences of said probes being selected from x elements of one of the following sets:
   (SEQ ID NO: 62, SEQ ID NO: 63 or SEQ ID NO: 64)
   (SEQ ID NO: 65, SEQ ID NO: 66 or SEQ ID NO: 67)
   (SEQ ID NO: 68, SEQ ID NO: 69 or SEQ ID NO: 70)
   (SEQ ID NO: 71, SEQ ID NO: 72 or SEQ ID NO: 73)
   x being 2 or 3,
   or the sequences of said probes having at least 92% identity with the abovementioned sequences SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72 or SEQ ID NO: 73,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic algae of the genus Prorocentrum.

### Embodiment E

The invention also concerns a kit as described above according to the method of embodiment A, B, C or D, for the detection of active living cells of toxic algae of toxic algae of the genus Alexandrium and/or Chattonella, said kit containing in addition:
a) at least one pair of probes specific to toxic algae of the genus Chattonella, the sequences of said probes being selected from x elements of one of the following sets:
   (SEQ ID NO: 74, SEQ ID NO: 75 or SEQ ID NO: 76)
   (SEQ ID NO: 77, SEQ ID NO: 78 or SEQ ID NO: 79)
   (SEQ ID NO: 80, SEQ ID NO: 81 or SEQ ID NO: 82)
   x being 3,
   or the sequences of said probes having at least 92% identity with said sequences SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81 or SEQ ID NO: 82,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
      said capture probe and said signal probe being capable of hybridizing with the ribosomal nucleic acid of a toxic algae of the genus Chattonella.

### Embodiment F

The invention also concerns a kit as described above according to embodiments A, B, C, D or E, for the detection of active living cells of toxic algae of the genus Alexandrium and/or Gymnodinium, said kit containing in addition:
a) at least one pair of probes specific to toxic algae of the genus Gymnodinium, the sequences of said probes being selected from x elements of one of the following sets:
   (SEQ ID NO: 83, SEQ ID NO: 84 or SEQ ID NO: 85)
   (SEQ ID NO: 86, SEQ ID NO: 87 or SEQ ID NO: 88)
   (SEQ ID NO: 89, SEQ ID NO: 90 or SEQ ID NO: 91)
   (SEQ ID NO: 92, SEQ ID NO: 93 or SEQ ID NO: 94)
   x being 3,
   or the sequences of said probes having at least 92% identity with the abovementioned sequences SEQ ID NO: 83, SEQ ID NO: 84, SEQ ID NO: 85, SEQ ID NO: 6, SEQ ID NO: 87, SEQ ID NO: 88, SEQ ID NO: 89, SEQ ID NO: 90, SEQ ID NO: 91, SEQ ID NO: 92, SEQ ID NO: 93 or SEQ ID NO: 94,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic algae of the genus Gymnodinium.

### Embodiment G

The invention also concerns a kit as described above according to embodiments A, B, C, D, E or F, for the detection of active living cells of toxic algae of the genus Alexandrium and/or Karenia, said kit containing in addition:
a) at least one pair of probes specific to toxic algae of the genus Karenia, the sequences of said probes being selected from x elements of one of the following sets:
   (SEQ ID NO: 95, SEQ ID NO: 96 or SEQ ID NO: 97)
   (SEQ ID NO: 98, SEQ ID NO: 99 or SEQ ID NO: 100)
   (SEQ ID NO: 101, SEQ ID NO: 102 or SEQ ID NO: 103)
   (SEQ ID NO: 104, SEQ ID NO: 105 or SEQ ID NO: 106)
   (SEQ ID NO: 107, SEQ ID NO: 108 or SEQ ID NO: 109)
   (SEQ ID NO: 110, SEQ ID NO: 111 or SEQ ID NO: 112)
   (SEQ ID NO: 113, SEQ ID NO: 114 or SEQ ID NO: 115)
   (SEQ ID NO: 116, SEQ ID NO: 117 or SEQ ID NO: 118)
   x being 3,
   or the sequences of said probes having at least 92% identity with the abovementioned sequences SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, SEQ ID NO: 114, SEQ ID NO: 115, SEQ ID NO: 116, SEQ ID NO: 117 or SEQ ID NO: 118,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic algae of the genus Karenia.

### Embodiment H

The invention also concerns a kit as described above according to embodiments A, B, C, D, E, F or G, for the detection of active living cells of toxic algae of the genus Alexandrium and/or Lingulodinium, said kit containing in addition:
a) at least one pair of probes specific to toxic algae of the genus Lingulodinium, the sequences of said probes being selected from x elements of one of the following sets:
   (SEQ ID NO: 119, SEQ ID NO: 120 or SEQ ID NO: 121)
   (SEQ ID NO: 122, SEQ ID NO: 123 or SEQ ID NO: 124)
   (SEQ ID NO: 125, SEQ ID NO: 126 or SEQ ID NO: 127)
   x being 3,
   or the sequences of said probes having at least 92% identity with the abovementioned sequences SEQ ID NO: 119, SEQ ID NO: 120, SEQ ID NO: 121, SEQ ID NO: 122, SEQ ID NO: 123, SEQ ID NO: 124, SEQ ID NO: 125, SEQ ID NO: 126 or SEQ ID NO: 127,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic algae of the genus Lingulodinium.

### Embodiment I

The invention also concerns a kit as described above according to embodiments A, B, C, D, E, F, G or H, for the detection of active living cells of toxic algae of the genus Alexandrium and/or Heterosigma, said kit containing in addition:
a) at least one pair of probes specific to toxic algae of the genus Heterosigma, the sequences of said probes being selected from x elements of one of the following sets:
   (SEQ ID NO: 128 and SEQ ID NO: 129)
   x being 2,
   or the sequences of said probes having at least 92% identity with the aforementioned sequences SEQ ID NO: 128 or SEQ ID NO: 129,
   one probe of said pair being a capture probe linked to at least one attachment molecule positioned at 3′ or 5′ of its sequence and the other probe of said pair being a signal probe linked to at least one marking molecule positioned at 3′ or 5′ of its sequence,
   said capture probe and said signal probe being capable of hybridizing with the ribosomal nucleic acid of a toxic algae of the genus Heterosigma.

As with the first aspect relating to use, all combinations of embodiments A, B, C, D, E, F, G, H and/or I of this fifth aspect can be considered. In this way, all combinations of toxic algae B to 1128 described in the first aspect can be detected by the kits as described in this fifth aspect.

In all embodiments of this fifth aspect and according to a particular embodiment, said capture probe is linked to at least one attachment molecule positioned at 5′ of its sequence and said signal probe is linked to at least one marking molecule positioned at 5′ of its sequence.

In all embodiments of this fifth aspect and according to another particular embodiment, said capture probe is linked to at least one attachment molecule positioned at 5′ of its sequence and said signal probe is linked to at least one marking molecule positioned at 3′ of its sequence.

In all embodiments of this fifth aspect and according to another particular embodiment, said capture probe is linked to at least one attachment molecule positioned at 3′ of its sequence and said signal probe is linked to at least one marking molecule positioned at 5′ of its sequence.

In all embodiments of this fifth aspect and according to another particular embodiment, said capture probe is linked to an attachment molecule positioned at 3′ of its sequence and said signal probe is linked to one or more marking molecules positioned at 3′ of its sequence.

In all embodiments of this fifth aspect, the said “at least one attachment molecule” can be chosen from a biotin molecule, avidin, streptavidin, a thiol group, an amine group and a carbon group.

In all embodiments of this fifth aspect and in a particular embodiment, the said “at least one attachment molecule” is a biotin molecule.

In all embodiments of this fifth aspect, the said “at least one marking molecule” may be chosen from a fluorochrome, a biotin, a molecule linked to a biotin, digoxigenin, an enzyme using a chemiluminescent substrate, an enzyme using a chromogenic substrate or an enzyme using an electrochemical oxidation substrate.

In all embodiments used to achieve this fifth aspect and in one particular embodiment, the said at least one marking molecule is digoxigenin.

In all embodiments of this fifth aspect, the said fluorochrome can be chosen from the group consisting of: Alexa fluor, in particular Alexa fluor 350, 405, 430, 488, 500, 514, 532, 546, 555, 568, 594, 610, 633, 647, 660, 680, 700, 750 or 790, Fluorescein Isothiocyanate (FITC), Rhodamine, Allophycocyanine (APC) and Phycoerythrin (PE).

In all embodiments of this fifth aspect, said enzyme using a chemiluminescent substrate may be horseradish peroxidase (HRP) and said chemiluminescent substrate may be luminol, or said enzyme using a chemiluminescent substrate may be luciferase and said chemiluminescent substrate may be luciferin.

In all embodiments of this fifth aspect, said enzyme using a chromogenic substrate may be alkaline phosphatase and said chromogenic substrate may be Tetrazolium nitroblue (NBT) or bromochlorylindolophosphate (BCIP), or said enzyme using a chromogenic substrate may be horseradish peroxidase (HRP) and said chromogenic substrate may be selected from 3,3′-Diaminobenzidine (DAB), 3,3′-Diaminobenzidine (DAB), 3,3′-Diaminobenzidine (DAB), 3,3′-Diaminobenzidine (DAB), 3,3′-Diaminobenzidine (DAB) and 3,3′-Diaminobenzidine (DAB). 3,3′,5,5′-Tetramethylbenzidine (TMB), or 2,2′-azino-bis(3-ethylbenzothiazoline-6-sulphonic acid) (ABTS).

In all embodiments of this fifth aspect, said enzyme using an electrochemically oxidized substrate can be horseradish peroxidase (HRP) and said electrochemically oxidized substrate can be 3,3′,5,5′-Tetramethylbenzidine (TMB).

In all embodiments of this fifth aspect and in a particular method of carrying out, the said hybridization solution may comprise 0 to 0.3 M NaCl, 0 to 0.1 M buffer chosen from citrate, Tris-HCl, PIPES, HEPES or phosphate, 0.001 to 0.05% detergent agent chosen from SDS, Triton®, TWEEN®20, optionally 0.001 to 0.5 M chelating agent selected from EDTA or EGTA, optionally 0.1 to 30% blocking agent selected from BSA, herring DNA, salmon DNA, calf DNA, yeast DNA or an exogenous protein and optionally another chemical agent selected from MgCl₂, CaCl₂ and KCl, preferably MgCl₂.

In all embodiments of this fifth aspect and in another particular embodiment, said hybridization solution may comprise 0.1 M to 1 M of NaCl or KCl, 0.01 M to 1 M of Tris-HCl, HEPES, PBS, KH₂PO₄ or SSC with a pH ranging from 6.0 to 9.0, 0.01 and 0.05% of detergent agent selected from SDS or N-Lauroylsarcosine, optionally 0.01 and 0.1 M chelating agent selected from EDTA, EGTA or a similar chelating agent selected from calcium citrate or sodium hexametaphosphate and optionally 0.1 and 30% blocking agent selected from a protein such as Bovine Serum Albumin Protein (BSA) or a nucleic acid such as Herring DNA.

In all embodiments of this fifth aspect and in another particular embodiment, the said hybridization solution may consist of 0.3 M of NaCl, 0.08 M of Tris-HCl and 0.04% of SDS and is pH 8.

In all embodiments of this fifth aspect and in a particular embodiment, the said washing solution may comprise 0 to 0.3 M NaCl, 0 to 0.1 M buffer chosen from citrate, Tris-HCl, PIPES, HEPES or phosphate, 0.001 to 0.05% detergent agent chosen from SDS, Triton®, TWEEN®20, optionally 0.001 to 0.5 M chelating agent selected from EDTA or EGTA, optionally 0.1 to 30% blocking agent selected from BSA, herring DNA, salmon DNA, calf DNA, yeast DNA or an exogenous protein and optionally another chemical agent selected from MgCl₂, CaCl₂) and KCl, preferably MgCl₂.

In all embodiments of this fifth aspect and in another particular embodiment, said washing solution may comprise 0.1 M to 1 M of NaCl or KCl, 0.01 M to 1 M of Tris-HCl, HEPES, PBS, KH₂PO₄ or SSC with a pH ranging from 6.0 to 9.0, 0.01 and 0.05% of detergent agent selected from SDS or N-Lauroylsarcosine, optionally 0.01 and 0.1 M chelating agent selected from EDTA, EGTA or a similar chelating agent selected from calcium citrate or sodium hexametaphosphate and optionally 0.1 and 30% blocking agent selected from a protein such as Bovine Serum Albumin Protein (BSA) or a nucleic acid such as Herring DNA.

In all embodiments of this fifth aspect and in another particular embodiment, the said washing solution may comprise 0.01 and 0.7 M of PBS, Na₂HPO₄, KH₂PO₄, K₂PO₄ and/or SSC, and 0.1 and 0.4 M of NaCl or KCl.

In all embodiments of this fifth aspect and in another particular embodiment, the said washing solution may consist of 0.1M K₂PO₄, 0.1M KH₂PO₄ and 0.1M KCl and is pH 7.6.

In all embodiments of this fifth aspect, the term “revelation solution” means any solution containing the means necessary for the revelation of any hybridization between the capture probe, the ribosomal nucleic acid and the signal probe. Depending on the labelling molecule used, the kit of the present invention may include one or more revelation solutions.

For example, when the marker molecule is a biotin molecule or is conjugated with a biotin molecule, the developer solution may contain a fluorochrome conjugated with streptavidin or avidin.

For example, when the marker molecule is a digoxigenin molecule, the developer solution may contain a fluorochrome conjugated to an anti-digoxigenin antibody.

For example, when the marker molecule is an enzyme using a chemiluminescent substrate, such as horseradish peroxidase or luciferase, the developer solution may contain the corresponding chemiluminescent substrate, such as luminol when the enzyme using a chemiluminescent substrate is horseradish peroxidase or luciferin when the enzyme using a chemiluminescent substrate is luciferase.

For example, when the marker molecule is an enzyme using a chromogenic substrate, such as alkaline phosphatase or horseradish peroxidase, the developer solution may contain the corresponding chromogenic substrate, such as Tetrazolium Nitroblue (NBT) or Bromochlorylindolophosphate (BCIP) when the enzyme using a chromogenic substrate is alkaline phosphatase or 3,3′-Diaminobenzidine (DAB), 3,3′,5,5′-Tetramethylbenzidine (TMB), or 2,2′-azino-bis(3-ethylbenzothiazoline-6-sulphonic acid) (ABTS) when the enzyme using a chemiluminescent substrate is horseradish peroxidase.

For example, when the marker molecule is an enzyme using an electrochemically oxidised substrate, such as horseradish peroxidase, the developer solution may contain the electrochemically oxidised substrate, such as 3,3′,5,5′-Tetramethylbenzidine (TMB).

For example, when the marker molecule is a biotin molecule or is conjugated with a biotin molecule, the kit of the present invention may include two development solutions.

For example, one of the disclosure solutions may include:
an enzyme using a chemiluminescent substrate, such as horseradish peroxidase or luciferase, coupled with streptavidin or avidin, or
an enzyme using a chromogenic substrate, such as alkaline phosphatase or horseradish peroxidase, coupled with streptavidin or avidin, or
an enzyme using an electrochemically oxidised substrate, such as horseradish peroxidase.

The alternative disclosure solution may include:
a chemiluminescent substrate, such as luminol when the enzyme using a chemiluminescent substrate is horseradish peroxidase or luciferin when the enzyme using a chemiluminescent substrate is luciferase, or
a chromogenic substrate, such as Tetrazolium Nitroblue (NBT) or Bromochlorylindolophosphate (BCIP) when the enzyme using a chromogenic substrate is alkaline phosphatase or 3,3′-Diaminobenzidine (DAB), 3,3′,5,5′-Tetramethylbenzidine (TMB), or 2,2′-azino-bis(3-ethylbenzothiazoline-6-sulphonic acid) (ABTS) when the enzyme using a chemiluminescent substrate is horseradish peroxidase, or
an electrochemically oxidised substrate, such as 3,3′,5,5′-Tetramethylbenzidine (TMB).

For example, when the marking molecule is a digoxigenin molecule the kit of the present invention may include two revealing solutions.

One of the revelation solutions may, for example, contain:
an enzyme using a chemiluminescent substrate conjugated to an anti-digoxigenin antibody, or
an enzyme using a chromogenic substrate conjugated to an anti-digoxigenin antibody, or
an enzyme using an electrochemically oxidised substrate conjugated to an anti-digoxigenin antibody, or

The alternative disclosure solution may include:
a chemiluminescent substrate, such as luminol when the enzyme using a chemiluminescent substrate is horseradish peroxidase or luciferin when the enzyme using a chemiluminescent substrate is luciferase, or
a chromogenic substrate, such as Tetrazolium Nitroblue (NBT) or Bromochlorylindolophosphate (BCIP) when the enzyme using a chromogenic substrate is alkaline phosphatase or 3,3′-Diaminobenzidine (DAB), 3,3′,5,5′-Tetramethylbenzidine (TMB), or 2,2′-azino-bis(3-ethylbenzothiazoline-6-sulphonic acid) (ABTS) when the enzyme using a chemiluminescent substrate is horseradish peroxidase, or
an electrochemically oxidised substrate, such as 3,3′,5,5′-Tetramethylbenzidine (TMB).

In all embodiments of this fifth aspect and in a particular embodiment, the said kit can also include a lysis solution.

According to this particular embodiment, the said lysis solution may comprise a neutral buffer chosen from phosphate, SSC or Tris, a chaotropic agent chosen from guanidium chloride, an ionic or non-ionic detergent such as sodium dodecyl sulphate (SDS) or Triton® X100, a reducing agent selected from b-mercaptoethanol or DiThioTreitol and a chelating agent selected from Ethylene Diamine Tetra Acetic Acid (EDTA) or Ethylene Glycol Tetra Acetic Acid (EGTA).

In all embodiments of this fifth aspect and in a particular embodiment, the said kit can include in addition a chromogenic substrate when:
the marking molecule is an enzyme using a chromogenic substrate,
the marker molecule is biotin and is detected via an enzyme using a chromogenic substrate conjugated to streptavidin or avidin
the marker molecule is conjugated to biotin and is detected via an enzyme using a chromogenic substrate conjugated to streptavidin or avidin, or
the marker molecule is digoxigenin and is detected via an enzyme using a chromogenic substrate conjugated to an anti-digoxigenin antibody.

According to this particular embodiment, said enzyme using a chromogenic substrate can be alkaline phosphatase and said chromogenic substrate can be Tetrazolium Nitroblue (NBT) or Bromochlorylindolophosphate (BCIP), or said enzyme using a chromogenic substrate may be horseradish peroxidase (HRP) and said chromogenic substrate may be selected from 3,3′-Diaminobenzidine (DAB), 3,3′,5,5′-Tetramethylbenzidine (TMB), or 2,2′-azino-bis(3-ethylbenzothiazoline-6-sulphonic acid) (ABTS).

In all embodiments of this fifth aspect and in a particular embodiment, the said kit can include in addition a chemiluminescent substrate when:
the marking molecule is an enzyme using a chemiluminescent substrate
the marker molecule is biotin and is detected via an enzyme using a chemiluminescent substrate conjugated to streptavidin or avidin
the marker molecule is conjugated to biotin and is detected via an enzyme using a chemiluminescent substrate conjugated to streptavidin or avidin, or
the marker molecule is digoxigenin and is detected via an enzyme using a chemiluminescent substrate conjugated to an anti-digoxigenin antibody.

According to this particular embodiment, said enzyme using a chemiluminescent substrate may be horseradish peroxidase (HRP) and said chemiluminescent substrate may be luminol, or said enzyme using a chemiluminescent substrate may be luciferase and said chemiluminescent substrate may be luciferin.

In all embodiments of this fifth aspect and in a particular embodiment, the said kit can include in addition an electrochemical oxidation substrate when:
the marking molecule is an enzyme using an electrochemical oxidation substrate
the marker molecule is biotin and is detected via an enzyme using an electrochemically oxidised substrate conjugated to streptavidin or avidin
the marker molecule is conjugated to biotin and is detected via an enzyme using an electrochemically oxidised substrate conjugated to streptavidin or avidin, or
the marker molecule is digoxigenin and is detected via an enzyme using an electrochemically oxidised substrate conjugated to an anti-digoxigenin antibody.

According to this particular embodiment, said enzyme using an electrochemically oxidizable substrate may be horseradish peroxidase (HRP) and said electrochemically oxidizable substrate may be 3,3′,5,5′-Tetramethylbenzidine (TMB).

In all embodiments of this fifth aspect and in a particular embodiment, the said kit may also include a solution containing an anti-digoxigenin antibody when the labelling molecule is digoxigenin.

According to this particular embodiment, the said anti-digoxigenin antibody can be conjugated:
to a fluorochrome
to an enzyme using a chromogenic substrate
to an enzyme using a chemiluminescent substrate
to an enzyme using an electrochemical oxidation substrate.

In all embodiments of this fifth aspect, the said fluorochrome can be chosen from the group consisting of: Alexa fluor, in particular Alexa fluor 350, 405, 430, 488, 500, 514, 532, 546, 555, 568, 594, 610, 633, 647, 660, 680, 700, 750 or 790, Fluorescein Isothiocyanate (FITC), Rhodamine, Allophycocyanine (APC) and Phycoerythrin (PE).

In all embodiments of this fifth aspect, said enzyme using a chromogenic substrate may be alkaline phosphatase and said chromogenic substrate may be Tetrazolium nitroblue (NBT) or bromochlorylindolophosphate (BCIP), or said enzyme using a chromogenic substrate may be horseradish peroxidase (HRP) and said chromogenic substrate may be selected from 3,3′-Diaminobenzidine (DAB), 3,3′-Diaminobenzidine (DAB), 3,3′-Diaminobenzidine (DAB) and 3,3′-Diaminobenzidine (DAB). 3,3′,5,5′-Tetramethylbenzidine (TMB), or 2,2′-azino-bis(3-ethylbenzothiazoline-6-sulphonic acid) (ABTS).

In all embodiments of this fifth aspect, said enzyme using a chemiluminescent substrate may be horseradish peroxidase (HRP) and said chemiluminescent substrate may be luminol, or said enzyme using a chemiluminescent substrate may be luciferase and said chemiluminescent substrate may be luciferin.

In all embodiments of this fifth aspect, said enzyme using an electrochemically oxidized substrate can be horseradish peroxidase (HRP) and said electrochemically oxidized substrate can be 3,3′,5,5′-Tetramethylbenzidine (TMB).

In all embodiments of this fifth aspect, and according to a particular embodiment, the said kit can also include a support.

According to this particular embodiment, the said support can be chosen from the group consisting of: a microplate, a glass slide, magnetic beads, electrodes printed in different materials such as carbon or gold.

According to this particular embodiment, the said support can be functionalized with streptavidin, avidin, an aldehyde group, an epoxy group, a carboxyl group, an isothiocyanate group, gold, mercaptosilane or a maleimide group.

Thus, the invention also relates to a kit as described above, said kit comprising in addition a support,

said support being notably selected from the group consisting of: a microplate, a glass slide, magnetic beads, electrodes printed in different materials such as carbon or gold, preferably a microplate or magnetic balls.

In all embodiments of this fifth aspect, and according to a particular embodiment, said kit may additionally include a positive control, the positive control being a synthetic nucleic acid molecule complementary to said signal probe and said capture probe.

In all embodiments of this fifth aspect, and according to a particular embodiment, said kit may additionally include a negative control, the negative control being a synthetic nucleic acid molecule non-complementary to said signal probe and said capture probe.

In all embodiments of this fifth aspect and in a particular embodiment, the said signal probes can be kept in one of the hybridization solutions as defined above.

In all embodiments of this fifth aspect, and in a particular embodiment, the said captured probes can be kept on a support as previously defined.

In all embodiments of this fifth aspect, and in a particular embodiment, the said support containing the said captured probes can be preserved in a conservation solution such as, for example, the commercial ProClin® solution (Sigma-Aldrich®, 48912-U).

In all embodiments of this fifth aspect, and in another particular embodiment, the said support containing the said captured probes can preferably be preserved freeze-dried on the support.

In a particular aspect of the invention, the said kit is preferably stored at 4° C.

In all embodiments of this fifth aspect, and in a particular embodiment, the said kit can contain in addition a procedure for using the said kit.

Another embodiment of this fifth aspect concerns a kit for the detection of active live cells of toxic algae of the genus Alexandrium, said kit containing:
a) at least one pair of probes specific to toxic algae of the genus Alexandrium, the sequences of said probes being selected from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22)
   (SEQ ID NO: 23, SEQ ID NO: 24 or SEQ ID NO: 25)
   (SEQ ID NO: 26, SEQ ID NO: 27 or SEQ ID NO: 28)
   x being 2 or 3,
   or the sequences of said probes having at least 92% identity with the abovementioned sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28,
   one probe of said pair being a capture probe linked to at least one attachment molecule positioned at 3′ or 5′ of its sequence and the other probe of said pair being a signal probe linked to at least one signal molecule positioned at 3′ or 5′ of its sequence,
   said capture probe and said signal probe being capable of hybridizing with the ribosomal nucleic acid of a toxic algae of the genus Alexandrium,
   said attachment molecule being a biotin molecule,
   said signal molecule being digoxigenin
b) a hybridization solution containing 0.3M NaCl, 0.08M Tris-HCl and 0.04% SDS and pH 8,
c) a washing solution containing 0.1M K₂PO₄, 0.1M KH₂PO₄ and 0.1M KCl and is pH 7.6,
d) a lysis solution being a commercial solution from the Quick-RNA™ MiniPrep kit (Zymo Research®, USA),
e) a support, said support being a microplate functionalized with streptavidin or avidin
f) a solution containing an anti-digoxigenin antibody, said anti-digoxigenin antibody being bound to horseradish peroxidase (HRP)
g) a chromogenic substrate, said chromogenic substrate being the 3.3′, 5, 5′-Tetramethylbenzidine (TMB),
h) a positive control, said positive control being a synthetic nucleic acid molecule complementary to said signal probe and said capture probe
i) a negative control, said negative control being a synthetic nucleic acid molecule non-complementary to said signal probe and said capture probe
   said captured probes being kept freeze-dried on said support,
   said signal probes being kept in said hybridization solution.

Another embodiment of this fifth aspect concerns a kit for the detection of active live cells of toxic algae of the genus Alexandrium, said kit containing:
a) at least one pair of probes specific to toxic algae of the genus Alexandrium, the sequences of said probes being selected from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22)
   (SEQ ID NO: 23, SEQ ID NO: 24 or SEQ ID NO: 25)
   (SEQ ID NO: 26, SEQ ID NO: 27 or SEQ ID NO: 28)
   x being 2 or 3,
   or the sequences of said probes having at least 92% identity with the abovementioned sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28,
   one probe of said pair being a capture probe linked to at least one attachment molecule positioned at 3′ or 5′ of its sequence and the other probe of said pair being a signal probe linked to at least one signal molecule positioned at 3′ or 5′ of its sequence,
   said capture probe and said signal probe being capable of hybridizing with the ribosomal nucleic acid of a toxic algae of the genus Alexandrium,
   said attachment molecule being a biotin molecule,
   said signal molecule being digoxigenin
b) at least one pair of probes specific to toxic algae of the genus Dinophysis, the sequences of said probes being selected from x elements of one of the following sets:
   (SEQ ID NO: 29, SEQ ID NO: 30 or SEQ ID NO: 31)
   (SEQ ID NO: 32 and SEQ ID NO: 33)
   (SEQ ID NO: 34 and SEQ ID NO: 35)
   (SEQ ID NO: 36 and SEQ ID NO: 37)
   (SEQ ID NO: 38 and SEQ ID NO: 39)
   (SEQ ID NO: 40, SEQ ID NO: 41 or SEQ ID NO: 42)
   (SEQ ID NO: 43, SEQ ID NO: 44 or SEQ ID NO: 45), or
   (SEQ ID NO: 46, SEQ ID NO: 47 or SEQ ID NO: 48)
   x being 2 or 3,
   or the sequences of said probes having at least 92% identity with the abovementioned sequences SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48,
   one probe of said pair being a capture probe linked to at least one attachment molecule positioned at 3′ or 5′ of its sequence and the other probe of said pair being a signal probe linked to at least one signal molecule positioned at 3′ or 5′ of its sequence,
   said capture probe and said signal probe being capable of hybridizing with the ribosomal nucleic acid of a toxic algae of the genus Dinophysis, said attachment molecule being a biotin molecule, said signal molecule being digoxigenin
c) a hybridization solution containing 0.3M NaCl, 0.08M Tris-HCl and 0.04% SDS and pH 8,
d) a washing solution containing 0.1 M K₂PO₄, 0.1 M KH₂PO₄ and 0.1 M KCl and is pH 7.6,
e) a lysis solution being a commercial solution from the Quick-RNA™ MiniPrep kit (Zymo Research®, USA),
f) a support, said support being a microplate functionalized with streptavidin or avidin
g) a solution containing an anti-digoxigenin antibody, said anti-digoxigenin antibody being bound to horseradish peroxidase (HRP)
h) a chromogenic substrate, said chromogenic substrate being the 3.3′, 5, 5′-Tetramethylbenzidine (TMB),
i) a positive control, said positive control being a synthetic nucleic acid molecule complementary to said signal probe and said capture probe
j) a negative control, said negative control being a synthetic nucleic acid molecule non-complementary to said signal probe and said capture probe
   said captured probes being kept freeze-dried on said support,
   said signal probes being kept in said hybridization solution.

Another embodiment of this fifth aspect concerns a kit for the detection of active live cells of toxic algae of the genus Alexandrium, said kit containing:
a) at least one pair of probes specific to toxic algae of the genus Alexandrium, the sequences of said probes being selected from x elements of one of the following sets:
   (SEQ ID NO: 1, SEQ ID NO: 2 or SEQ ID NO: 3)
   (SEQ ID NO: 4 and SEQ ID NO: 5)
   (SEQ ID NO: 6 and SEQ ID NO: 7)
   (SEQ ID NO: 8, SEQ ID NO: 9 or SEQ ID NO: 10)
   (SEQ ID NO: 11, SEQ ID NO: 12 or SEQ ID NO: 13)
   (SEQ ID NO: 14, SEQ ID NO: 15 or SEQ ID NO: 16)
   (SEQ ID NO: 17, SEQ ID NO: 18 or SEQ ID NO: 19)
   (SEQ ID NO: 20, SEQ ID NO: 21 or SEQ ID NO: 22)
   (SEQ ID NO: 23, SEQ ID NO: 24 or SEQ ID NO: 25)
   (SEQ ID NO: 26, SEQ ID NO: 27 or SEQ ID NO: 28)
   x being 2 or 3,
   or the sequences of said probes having at least 92% identity with the abovementioned sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28,
   one probe of said pair being a capture probe linked to at least one attachment molecule positioned at 3′ or 5′ of its sequence and the other probe of said pair being a signal probe linked to at least one signal molecule positioned at 3′ or 5′ of its sequence,
   said capture probe and said signal probe being capable of hybridizing with the ribosomal nucleic acid of a toxic algae of the genus Alexandrium, said attachment molecule being a biotin molecule, said signal molecule being digoxigenin
b) at least one pair of probes specific to toxic algae of the genus Dinophysis, the sequences of said probes being selected from x elements of one of the following sets:
   (SEQ ID NO: 29, SEQ ID NO: 30 or SEQ ID NO: 31)
   (SEQ ID NO: 32 and SEQ ID NO: 33)
   (SEQ ID NO: 34 and SEQ ID NO: 35)
   (SEQ ID NO: 36 and SEQ ID NO: 37)
   (SEQ ID NO: 38 and SEQ ID NO: 39)
   (SEQ ID NO: 40, SEQ ID NO: 41 or SEQ ID NO: 42),
   (SEQ ID NO: 43, SEQ ID NO: 44 or SEQ ID NO: 45), or
   (SEQ ID NO: 46, SEQ ID NO: 47, SEQ ID NO: 48)
   x being 2 or 3,
   or the sequences of said probes having at least 92% identity with the abovementioned sequences SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48,
   one probe of said pair being a capture probe linked to at least one attachment molecule positioned at 3′ or 5′ of its sequence and the other probe of said pair being a signal probe linked to at least one signal molecule positioned at 3′ or 5′ of its sequence,
   said capture probe and said signal probe being capable of hybridizing with the ribosomal nucleic acid of a toxic algae of the genus Dinophysis, said attachment molecule being a biotin molecule, said signal molecule being digoxigenin
c) at least one pair of probes specific to toxic algae of the genus Pseudo-nitzschia, the sequences of said probes being selected from x elements of one of the following sets:
   (SEQ ID NO: 49 and SEQ ID NO: 50)
   (SEQ ID NO: 51 and SEQ ID NO: 52)
   (SEQ ID NO: 53, SEQ ID NO: 54 or SEQ ID NO: 55)
   (SEQ ID NO: 56, SEQ ID NO: 57, SEQ ID NO: 58)
   (SEQ ID NO: 59, SEQ ID NO: 60, SEQ ID NO: 61)
   x being 2 or 3,
   or the sequences of said probes having at least 92% identity with the abovementioned sequences SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61,
   one probe of said pair being a capture probe linked to at least one attachment molecule positioned at 3′ or 5′ of its sequence and the other probe of said pair being a signal probe linked to at least one signal molecule positioned at 3′ or 5′ of its sequence,
   said capture probe and said signal probe being capable of hybridizing with the ribosomal nucleic acid of a toxic algae of the genus Pseudo-nitzschia, said attachment molecule being a biotin molecule, said signal molecule being digoxigenin
d) a hybridization solution containing 0.3 M NaCl, 0.08 M Tris-HCl and 0.04% SDS and pH 8,
e) a washing solution containing 0.1 M K₂PO₄, 0.1 M KH₂PO₄ and 0.1 M KCl and is pH 7.6.
f) a lysis solution being a commercial solution from the Quick-RNA™ MiniPrep kit (Zymo Research®, USA),
g) a support, said support being a microplate functionalized with streptavidin or avidin
h) a solution containing an anti-digoxigenin antibody, said anti-digoxigenin antibody being bound to horseradish peroxidase (HRP)
i) a chromogenic substrate, said chromogenic substrate being the 3.3′, 5, 5′-Tetramethylbenzidine (TMB),
j) a positive control, said positive control being a synthetic nucleic acid molecule complementary to said signal probe and said capture probe
k) a negative control, said negative control being a synthetic nucleic acid molecule non-complementary to said signal probe and said capture probe
   said captured probes being kept freeze-dried on said support,
   said signal probes being kept in said hybridization solution.

Another embodiment of this fifth aspect concerns a kit for the detection of active living cells of toxic algae of toxic algae of the genus Dinophysis, said kit containing:
a) at least one pair of probes specific to toxic algae of the genus Dinophysis, the sequences of said probes being selected from x elements of one of the following sets:
   (SEQ ID NO: 29, SEQ ID NO: 30 or SEQ ID NO: 31)
   (SEQ ID NO: 32 and SEQ ID NO: 33)
   (SEQ ID NO: 34 and SEQ ID NO: 35)
   (SEQ ID NO: 36 and SEQ ID NO: 37)
   (SEQ ID NO: 38 and SEQ ID NO: 39)
   (SEQ ID NO: 40, SEQ ID NO: 41 or SEQ ID NO: 42)
   (SEQ ID NO: 43, SEQ ID NO: 44 or SEQ ID NO: 45), or
   (SEQ ID NO: 46, SEQ ID NO: 47 or SEQ ID NO: 48)
   x being 2 or 3,
   or the sequences of said probes having at least 92% identity with the abovementioned sequences SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48,
   one probe of said pair being a capture probe linked to at least one attachment molecule positioned at 3′ or 5′ of its sequence and the other probe of said pair being a signal probe linked to at least one signal molecule positioned at 3′ or 5′ of its sequence,
   said capture probe and said signal probe being capable of hybridizing with the ribosomal nucleic acid of a toxic algae of the genus Dinophysis, said attachment molecule being a biotin molecule, said signal molecule being digoxigenin
b) a hybridization solution containing 0.3 M NaCl, 0.08 M Tris-HCl and 0.04% SDS and pH 8,
c) a washing solution containing 0.1 M K₂PO₄, 0.1 M KH₂PO₄ and 0.1 M KCl and is pH 7.6.
d) a lysis solution being a commercial solution from the Quick-RNA™ MiniPrep kit (Zymo Research®, USA),
e) a support, said support being a microplate functionalized with streptavidin or avidin
f) a solution containing an anti-digoxigenin antibody, said anti-digoxigenin antibody being bound to horseradish peroxidase (HRP)
g) a chromogenic substrate, said chromogenic substrate being the 3.3′, 5, 5′-Tetramethylbenzidine (TMB),
h) a positive control, said positive control being a synthetic nucleic acid molecule complementary to said signal probe and said capture probe
i) a negative control, said negative control being a synthetic nucleic acid molecule non-complementary to said signal probe and said capture probe
   said captured probes being kept freeze-dried on said support,
   said signal probes being kept in said hybridization solution.

Another embodiment of this fifth aspect concerns a kit for the detection of active live cells of toxic algae of toxic algae of the genus Pseudo-nitzschia, said kit containing:
a) at least one pair of probes specific to toxic algae of the genus Pseudo-nitzschia, the sequences of said probes being selected from x elements of one of the following sets:
   (SEQ ID NO: 49 and SEQ ID NO: 50)
   (SEQ ID NO: 51 and SEQ ID NO: 52)
   (SEQ ID NO: 53, SEQ ID NO: 54 or SEQ ID NO: 55)
   (SEQ ID NO: 56, SEQ ID NO: 57, SEQ ID NO: 58)
   (SEQ ID NO: 59, SEQ ID NO: 60, SEQ ID NO: 61)
   x being 2 or 3,
   or the sequences of said probes having at least 92% identity with the abovementioned sequences SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61,
   one probe of said pair being a capture probe linked to at least one attachment molecule positioned at 3′ or 5′ of its sequence and the other probe of said pair being a signal probe linked to at least one signal molecule positioned at 3′ or 5′ of its sequence,
   said capture probe and said signal probe being capable of hybridizing with the ribosomal nucleic acid of a toxic algae of the genus Pseudo-nitzschia,
   said attachment molecule being a biotin molecule,
   said signal molecule being digoxigenin
b) a hybridization solution containing 0.3 M NaCl, 0.08 M Tris-HCl and 0.04% SDS and pH 8,
c) a washing solution containing 0.1 M K₂PO₄, 0.1 M KH₂PO₄ and 0.1 M KCl and is pH 7.6.
d) a lysis solution being a commercial solution from the Quick-RNA™ MiniPrep kit (Zymo Research®, USA),
e) a support, said support being a microplate functionalized with streptavidin or avidin
f) a solution containing an anti-digoxigenin antibody, said anti-digoxigenin antibody being bound to horseradish peroxidase (HRP)
g) a chromogenic substrate, said chromogenic substrate being the 3.3′, 5, 5′-Tetramethylbenzidine (TMB),
h) a positive control, said positive control being a synthetic nucleic acid molecule complementary to said signal probe and said capture probe
i) a negative control, said negative control being a synthetic nucleic acid molecule non-complementary to said signal probe and said capture probe
   said captured probes being kept freeze-dried on said support,
   said signal probes being kept in said hybridization solution.

A sixth aspect of the invention concerns devices for the detection of active living cells of toxic algae.

### Embodiment A

Thus, according to this sixth aspect, the invention concerns a device consisting of a support comprising specific probes of toxic algae of the genus Alexandrium for the implementation of a method for the detection of active living cells of toxic algae in a sample likely to contain at least one toxic algae of the genus Alexandrium, said probes having a sequence selected from the sequences:

SEQ ID NO: 1, SEQ ID NO: 2, SEQ ID NO: 3, SEQ ID NO: 4, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28 or sequences having at least 92% identity with the above SEQ ID NO: 1, SEQ ID NO: 2, SEQ ID NO: 3, SEQ ID NO: 4, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 17, SEQ ID NO: 19, SEQ ID NO: 20, SEQ ID NO: 22, SEQ ID NO: 23, SEQ ID NO: 25, SEQ ID NO: 26, SEQ ID NO: 28,

each probe being linked or capable of being linked to at least one attachment molecule positioned at 3′ or 5′ of its sequence,
said probes being capable of hybridizing with the ribosomal nucleic acid of a toxic alga of the genus Alexandrium optionally present in said sample to form a complex.

### Embodiment B

In the same way, the invention also concerns a device such as described above according to embodiment A, comprising in addition specific probes of toxic algae of the genus Dinophysis for the implementation of a method for the detection of active living cells of toxic algae in a sample likely to contain at least one toxic algae of the genus Alexandrium and/or Dinophysis, said probes having a sequence selected from the sequences:

SEQ ID NO: 30, SEQ ID NO: 31, SEQ ID NO: 33, SEQ ID NO: 35, SEQ ID NO: 37, SEQ ID NO: 38, SEQ ID NO: 39, SEQ ID NO: 41, SEQ ID NO: 42, SEQ ID NO: 44, SEQ ID NO: 45, SEQ ID NO: 47, SEQ ID NO: 48 or the sequence of said probe having at least 92% identity with said sequences SEQ ID NO: 30, SEQ ID NO: 31, SEQ ID NO: 33, SEQ ID NO: 35, SEQ ID NO: 37, SEQ ID NO: 38, SEQ ID NO: 39, SEQ ID NO: 41, SEQ ID NO: 42, SEQ ID NO: 44, SEQ ID NO: 45, SEQ ID NO: 47, SEQ ID NO: 48

each probe being linked or capable of being linked to at least one attachment molecule positioned at 3′ or 5′ of its sequence,
said probes being capable of hybridizing with the ribosomal nucleic acid of a toxic alga of the genus Dinophysis optionally present in said sample to form a complex.

### Embodiment C

In the same way, the invention also concerns one of the devices as described above according to embodiment A or embodiment B, comprising in addition probes specific to toxic algae of the genus Pseudo-nitzschia for carrying out a method for detecting active living cells of toxic algae in a sample likely to contain at least one toxic alga of the genus Alexandrium and/or Pseudo-nitzschia, said probes having a sequence selected from the sequences:

SEQ ID NO: 49, SEQ ID NO: 51, SEQ ID NO: 52, SEQ ID NO: 54, SEQ ID NO: 55, SEQ ID NO: 57, SEQ ID NO: 58, SEQ ID NO: 60, SEQ ID NO: 61 or the sequence of said probe having at least 92% identity with said SEQ ID NO: 49, SEQ ID NO: 51, SEQ ID NO: 52, SEQ ID NO: 54, SEQ ID NO: 55, SEQ ID NO: 57, SEQ ID NO: 58, SEQ ID NO: 60, SEQ ID NO: 61

each probe being linked or capable of being linked to at least one attachment molecule positioned at 3′ or 5′ of its sequence,
said probes being capable of hybridizing with the ribosomal nucleic acid of a toxic alga of the genus Pseudo-nitzschia optionally present in said sample to form a complex.

### Embodiment D

In the same way, the invention also concerns one of the devices as described above according to the embodiment A, B or C, comprising in addition specific probes of toxic algae of the genus Prorocentrum for the implementation of a method for the detection of active living cells of toxic algae in a sample likely to contain at least one toxic algae of the genus Alexandrium and/or Prorocentrum, the said probes having a sequence chosen from the sequences:

SEQ ID NO: 63, SEQ ID NO: 64, SEQ ID NO: 66, SEQ ID NO: 67, SEQ ID NO: 69, SEQ ID NO: 70, SEQ ID NO: 72, SEQ ID NO: 73 or the sequence of said probe having at least 92% identity with the above sequences SEQ ID NO: 63, SEQ ID NO: 64, SEQ ID NO: 66, SEQ ID NO: 67, SEQ ID NO: 69, SEQ ID NO: 70, SEQ ID NO: 72, SEQ ID NO: 73

each probe being linked or capable of being linked to at least one attachment molecule positioned at 3′ or 5′ of its sequence,
said probes being capable of hybridizing with the ribosomal nucleic acid of a toxic alga of the genus Prorocentrum optionally present in said sample to form a complex.

### Embodiment E

In the same way, the invention also concerns one of the devices as described above according to the Embodiment A, B, C or D, comprising in addition probes specific to toxic algae of the genus Chattonella for the implementation of a method for the detection of active living cells of toxic algae in a sample likely to contain at least one toxic algae of the genus Alexandrium and/or Chattonella, said probes having a sequence selected from the sequences:

SEQ ID NO: 75, SEQ ID NO: 76, SEQ ID NO: 77, SEQ ID NO: 79, SEQ ID NO: 80, SEQ ID NO: 82 or the sequence of said probe having at least 92% identity with the above-mentioned sequences SEQ ID NO: 75, SEQ ID NO: 76, SEQ ID NO: 77, SEQ ID NO: 79, SEQ ID NO: 80, SEQ ID NO: 82

each probe being linked or capable of being linked to at least one attachment molecule positioned at 3′ or 5′ of its sequence,
said probes being capable of hybridizing with the ribosomal nucleic acid of a toxic algae of the genus Chattonella optionally present in said sample to form a complex

### Embodiment F

In the same way, the invention also concerns one of the devices as described above according to the Embodiment A, B, C, D or E, comprising in addition probes specific to toxic algae of the genus Gymnodinium for the implementation of a method for the detection of active living cells of toxic algae in a sample likely to contain at least one toxic alga of the genus Alexandrium and/or Gymnodinium, said probes having a sequence selected from the sequences:
SEQ ID NO: 84, SEQ ID NO: 85, SEQ ID NO: 87, SEQ ID NO: 88, SEQ ID NO: 90, SEQ ID NO: 91, SEQ ID NO: 93, SEQ ID NO: 94 or the sequence of said probe having at least 92% identity with the abovementioned sequences SEQ ID NO: 84, SEQ ID NO: 85, SEQ ID NO: 87, SEQ ID NO: 88, SEQ ID NO: 90, SEQ ID NO: 91, SEQ ID NO: 93, SEQ ID NO: 94
   each probe being linked or capable of being linked to at least one attachment molecule positioned at 3′ or 5′ of its sequence,
   said probes being capable of hybridizing with the ribosomal nucleic acid of a toxic algae of the genus Gymnodinium optionally present in said sample to form a complex.

### Embodiment G

In the same way, the invention also concerns one of the devices as described above according to embodiments A, B, C, D, E or F, comprising in addition probes specific to toxic algae of the genus Karenia for the implementation of a method for the detection of active living cells of toxic algae in a sample likely to contain at least one toxic algae of the genus Alexandrium and/or Karenia, said probes having a sequence selected from the sequences:
SEQ ID NO: 96, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 109, SEQ ID NO: 111, SEQ ID NO: 112, SEQ ID NO: 114, SEQ ID NO: 115, SEQ ID NO: 117, SEQ ID NO: 118 or the sequence of said probe having at least 92% identity with the abovementioned sequences SEQ ID NO: 96, SEQ ID NO: 97, SEQ ID NO: 99, SEQ ID NO: 100, SEQ ID NO: 102, SEQ ID NO: 103, SEQ ID NO: 105, SEQ ID NO: 106, SEQ ID NO: 108, SEQ ID NO: 109, SEQ ID NO: 111, SEQ ID NO: 112, SEQ ID NO: 114, SEQ ID NO: 115, SEQ ID NO: 117, SEQ ID NO: 118
   each probe being linked or capable of being linked to at least one attachment molecule positioned at 3′ or 5′ of its sequence,
   said probes being capable of hybridizing with the ribosomal nucleic acid of a toxic algae of the genus Karenia optionally present in said sample to form a complex.

### Embodiment H

In the same way, the invention also concerns one of the devices as described above according to embodiments A, B, C, D, E, F or G, comprising in addition probes specific to toxic algae of the genus Lingulodinium for the implementation of a method for detecting active living cells of toxic algae in a sample likely to contain at least one toxic alga of the genus Alexandrium and/or Lingulodinium, the said probes having a sequence chosen from the sequences:
SEQ ID NO: 120, SEQ ID NO: 121, SEQ ID NO: 123, SEQ ID NO: 124, SEQ ID NO: 126, SEQ ID NO: 127 or the sequence of said probe having at least 92% identity with the above SEQ ID NO: 120, SEQ ID NO: 121, SEQ ID NO: 123, SEQ ID NO: 124, SEQ ID NO: 126, SEQ ID NO: 127
   each probe being linked or capable of being linked to at least one attachment molecule positioned at 3′ or 5′ of its sequence,
   said probes being capable of hybridizing with the ribosomal nucleic acid of a toxic algae of the genus Lingulodinium optionally present in said sample to form a complex.

### Embodiment I

In the same way, the invention also concerns one of the devices as described above according to embodiments A, B, C, D, E, F, G or H, comprising in addition probes specific to toxic algae of the genus Heterosigma for the implementation of a method for the detection of active living cells of toxic algae in a sample likely to contain at least one toxic alga of the genus Alexandrium and/or Heterosigma, the said probes having a sequence chosen from the sequences:
the sequences SEQ ID NO: 128 or the sequence of said probe having at least 92% identity with the above said sequences SEQ ID NO: 128
   each probe being linked or capable of being linked to at least one attachment molecule positioned at 3′ or 5′ of its sequence,
   said probes being capable of hybridizing with the ribosomal nucleic acid of a toxic alga of the genus Heterosigma which may be present in said sample to form a complex.

As with the first aspect relating to use, all combinations of embodiments A, B, C, D, E, F, G, H and/or I of this sixth aspect can be considered.

In all embodiments of this sixth aspect and according to a particular embodiment, the said attachment molecule is located 5′ from the sequence of the said probe.

In all embodiments of this sixth aspect and according to another particular embodiment, the said attachment molecule is located in 3′ of the sequence of the said probe.

In all embodiments of this sixth aspect, the said attachment molecule can be chosen from a biotin molecule, avidin, streptavidin, a thiol group, an amine group and a carbon.

In all embodiments of this sixth aspect and according to a particularly preferred embodiment, the said attachment molecule is a biotin molecule.

In all embodiments of this sixth aspect and according to a particular embodiment, the device can be chosen from the group consisting of: a microplate, a glass slide, magnetic beads, electrodes printed in different materials such as carbon or gold.

In all embodiments of this sixth aspect and according to a particular embodiment, the said device can be:
functionalized with streptavidin or avidin and said attachment molecule is a biotin
functionalized with an aldehyde group, an epoxy group, a carboxyl group or an isothiocyanate group and said attachment molecule is an amine or carbon group.
functionalized with gold, mercaptosilane or a maleimide moiety and said attachment molecule is a thiol group.

In all embodiments of this sixth aspect, the sample may be a sample of seawater, brackish water, culture media or microalgae culture produced for commercial purposes.

The following figures and examples will better illustrate the invention, without limiting its scope.

### FIGURES

FIG. 1: Changes in cell density (cells/mL) of an Alexandrium minutum culture over time under optimal growth conditions.

FIG. 2: Average concentration of total RNA content per cell expressed in ng of total RNA per cell obtained from optimal culture conditions, i.e. corresponding to the exponential phase of growth.

FIG. 3: Specificity test of probes targeting Lingulodinium polyedrum. Hybridization of 200 ng of RNA extracted from a non axenic culture of Lingulodinium polyedrum with the following pairs of probes: SEQ ID NO: 119 and SEQ ID NO: 120 (probe n.199/120), SEQ ID NO: 122 and SEQ ID NO: 123 (probe n.122/123) or SEQ ID NO: 125 and SEQ ID NO: 126 (probe n. 125/126), 0.1 μM positive control (PC, complementary synthetic DNA) or 0.1 μM negative control (NC, non-complementary synthetic DNA).

FIG. 4: Correspondence between RNA concentration (ng/μL) and absorbance at 630 nm: Alexandrium minutum and sequence probes SEQ ID NO: 7 and SEQ ID NO: 8 (A.); Alexandrium tamarense and sequence probes SEQ ID NO: 17 and SEQ ID NO: 18 (B.); Alexandrium ostenfeldii and sequence probes SEQ ID NO: 11 and SEQ ID NO: 12 (C.).); Dinophysis acuminata and sequence probes SEQ ID NO: 40 and SEQ ID NO: 41 (D.); Lingulodinium polyedrum and sequence probes SEQ ID NO: 119 and SEQ ID NO: 120 (E.); Gymnodinium catenatum and sequence probes SEQ ID NO: 83 and SEQ ID NO: 84 (F.); Chattonella subsalsa and sequence probes SEQ ID NO: 74 and SEQ ID NO: 75 (G.).

FIG. 5: Correspondence between RNA concentration and absorbance at 450 nm (optimised protocol): Alexandrium minutum and sequence probes SEQ ID NO: 7 and SEQ ID NO: 8 (A); Alexandrium ostenfeldii and sequence probes SEQ ID NO: 11 and SEQ ID NO: 12 (B); Pseudo-nitzschia and sequence probes SEQ ID NO: 46 and SEQ ID NO: 47 (C, D and E).

FIG. 6: Correspondence between estimated cell concentration (cells/L) and absorbance at 630 nm: Alexandrium minutum in stationary phase (AM—STAT) and Alexandrium minutum in exponential phase (AM—EXP).

FIG. 7: Correspondence between estimated cell concentration (cells/L) and absorbance at 450 nm: Dinophysis sp in an environmental sample naturally containing Dinophysis.

FIG. 8: Comparison of absorbances read at 450 nm and obtained with different volumes of developing solution (TMB) made with different buffers.

FIG. 9: Comparison of absorbance units at 630 nm and 450 nm from tests performed with RNAs obtained from known numbers of cells.

FIG. 10: Comparison of absorbances read at 450 nm and obtained by hybridization tests carried out with different buffers (optimised protocol).

FIG. 11. Comparison of Pseudo-nitzschia detection by microscopic cell counts and sandwich hybridization test signals read at 630 nm in environmental samples collected during natural growth. The results are reported per 1 L of seawater sample.

FIG. 12: Comparison of Alexandrium detection by microscopic cell counts and sandwich hybridization test signals read at 630 nm in environmental samples collected during natural growth. The results are related to 1 L of seawater sample.

FIG. 13: Comparison of Dinophysis detection by absorbance signals from the sandwich hybridization test read at 450 nm a) (optimized protocol) and by cell counts in microscopy b). The results are related to 1 L of sea water.

### EXAMPLES

The development of the invention used different kinds of samples and required different experimental approaches, which are defined below including their scope. The main results obtained are summarised in Table 1.

The Different Types of Samples

Samples from Cell Culture

The samples were obtained from cultures from international collections. These cultures were maintained under optimal conditions of development as indicated in EXAMPLE 1: Probe Validation, a) Culture conditions. These are microalgae populations that are sufficiently homogeneous to present synchronous growth phases and optimal physiological condition, i.e., biological material in maximum condition and quantity.

The sensitivity of the tests was optimal because the material obtained is relatively pure since it contains a large majority of the target cells. These samples were used in a first phase of method development, more particularly to frame the limits of action of the experiments and to determine the most interesting study windows.

Artificial Samples

The artificial samples were made from environmental water that does not have the microalgae of interest beforehand, and were supplemented with cells from a homogeneous culture. The cells of the culture have a chosen physiological state, e.g. an exponential growth phase state or a stationary phase state, and are mostly synchronised, i.e. they divide together at the same time. The assembly of the environmental water with a determined number of culture cells constitutes an artificial sample that allows the impact of the components frequently found in the environmental water to be taken into consideration.

The composition of environmental water depends on biotic and abiotic factors (organisms present, various organic materials, chemical or biological pollution, etc.).). The concentration of this water on filters of low porosity, for example 10 μM, will accumulate matter from whatever source. The extraction of genetic material and the co-purifications associated with this extraction will make up what is known as the “environmental matrix”. This matrix has an impact on the detection sensitivity of any molecular method and leads to a decrease in the signal sought. It was therefore essential to carry out a battery of experiments with these samples in order to integrate this unmanageable dimension which is completely absent from cell culture samples.

The assembly of an artificial sample, i.e. volume of water plus volume of cell culture, cannot be equated with the a posteriori assembly of independently extracted biological material, i.e. material extracted from the volume of water plus material extracted from the cell culture.

Natural Samples

Natural samples are samples from environments with natural blooms of the targeted microalgae. Heterogeneity appears at the level of the sample itself and at the level of the targeted microalgae.

In the environment, microalgae populations are heterogeneous in terms of diversity and physiological state. A population of a genus of microalgae may include several different species and may contain all the life stages of a cell. For example, a large number of cells may correspond to a proliferating population, therefore active, or a senescent population, therefore not very active. A small number of cells may be very active or dormant

Strictly speaking, natural samples are heterogeneous in terms of biological, chemical and geographical richness.

The variability in content of organisms and organic and inorganic matter is very large and random from one sample to another. This variability will have a direct impact on the degree of dilution of the targeted toxic micro-algae: the higher the density of living organisms and matter, the more diluted the target is.

The location of the sample in the water column will also influence the distribution of the targeted micro-algae. Some microalgae are preferentially found in the euphotic zone or surface water, others prefer the depth and still others actively move throughout the water column They are all subject to a circadian clock that will determine their location in the water column over the course of a day.

All these characteristics make the environmental sample very heterogeneous and very representative of reality.

The Different Types of Experiences

Calibration Curves from Culture RNAs

The first type of experiment was the realization of calibration curves using total RNAs from clonal but not axenic cultures of toxic microalgae.

The aim of these experiments was to define the window of detection and quantification of targeted RNAs using microalgae in active growth phase. The values obtained were used to establish the sensitivity, reproducibility and robustness of the colorimetric test and are references for the analysis of environmental samples.

Calibration Curves from Artificial Environmental Samples

The second type of experiment was the realization of calibration curves using natural samples artificially contaminated by cells from clonal but not axenic cultures of toxic microalgae.

These experiments made it optional to integrate the variability linked to the environmental dimension and to calibrate the impact of matrices of different and varied origins on the colorimetric test. As a result, the limits obtained are by definition lower than those obtained with culture samples.

Monitoring of Natural Environmental Samples

The third type of experiment was the application of the invention in a natural environment using natural samples naturally contaminated by toxic micro-algae.

These experiments have enabled us to validate the developments and improvements of the colorimetric test but also to demonstrate its applicability in the field in terms of robustness, reproducibility, reliability and sensitivity. This is a field application with a view to industrialisation.

The Main Results

TABLE 1
Comparison of sensitivity thresholds between the method
of the invention before and after are optimised according
to the type of samples and experiments carried out.

| | | | Experiments | |
|---|---|---|---|---|
| | | | conducted with | Follow-up of |
| | Experiences with | | natural samples | the field with |
| | crops | | supplemented | naturally |
| | | Total | with culture | contaminated |
| | ng | number of | cells | samples |
| LOD | total | cells | Cells/Liter | Cells/Litres |
| Method of the | 1 | 50 | 100-500 | 160 |
| invention before | | | | (Alexandrium) |
| optimisation | | | | and |
| | | | | 500 |
| | | | | (Pseudo- |
| | | | | nitzschia) |
| Method of the | 0.1 | 5 | <200 or | <2 |
| invention after | | | even <5 | (Dynophysis) |
| optimization | | | | |

### Example 1: Probe Validation

a) Growing Conditions

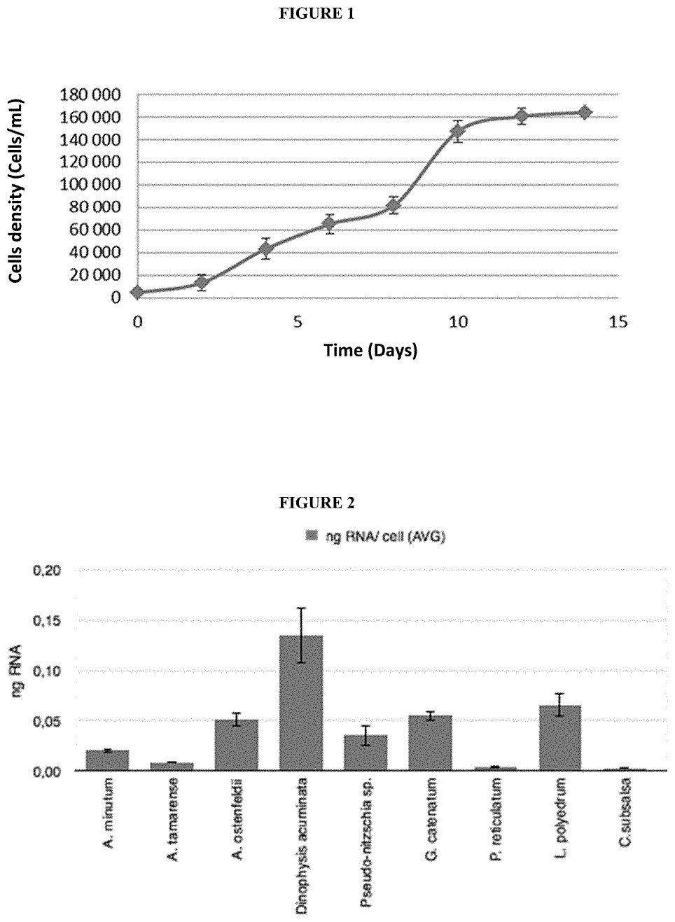

The cultures of algae used to test the probes described in this invention are listed in Table 2. All the cultures are currently maintained at Microbia Environnement on the business incubation site of the Oceanological Observatory of Banyuls-sur-mer, France. The cultures are maintained in seawater environments of type F/2 proposed by Guillard and Ryther (1962) and type L1 proposed by Guillard and Hargraves (1993) (at different temperatures and under a luminosity intensity of 100 μE. m-2.s-1 with a day:night cycle of 12:12. F/2 and L1 media are seawater-based media commonly used to grow seaweed and contain trace elements, vitamins and sometimes silica. The cells are counted every 2 days by flow cytometry using the FACSCanto® II flow cytometer (BD Biosciences®, USA). The counts show an exponential phase of growth (between day 2 and day 10) and a stationary phase from day 10 onwards (FIG. 1).

TABLE 2
Species for testing the probes described including
class, growth medium and strain number.

| | | Cultural | Strain |
|---|---|---|---|
| | | Environ- | identifica- |
| Micro-algae | Class | ment | tion number |
| Alexandrium minutum | Dinophyceae | F/2 | AM 205 |
| Alexandrium tamarense | Dinophyceae | F/2 | VGO928 |
| Alexandrium ostenfeldii | Dinophyceae | F/2 | VGO956 |
| Dinophysis acuminata | Dinophyceae | F/2 | VGO1063 |
| Dinophysis acuta | Dinophyceae | F/2 | VGO1065 |
| Pseudo-nitzschia artica | Bacillariophyceae | L1 | |
| Gymnodinium catenatum | Dinophyceae | L1 | GC12V |
| Lingulodinium polyedrum | Dinophyceae | L1 | GG1AM |
| Chattonella subsalsa | Raphidophyceae | L1 | CS0704 |

b) RNA Preparation

A known number of culture cells are either filtered on a polycarbonate membrane with a porosity of 3 μm (Whatman® Nuclepore Track-Etched Membranes) using a filtration system and a vacuum pump, or placed in a 15 mL tube and centrifuged at 5000 g for 8 minutes. The supernatant is removed by leaving approximately 2 mL of sample before being centrifuged again at 10,000 g for 1 minute to completely remove the remaining supernatant and preserve the cell pellet. 1 mL TRI-Reagent (Sigma®, France) or 1 mL Lysis Buffer from the Quick-RNA “MiniPrep kit (Zymo Research®, USA) is immediately added to each pellet or filtrate and homogenised. Cell lysis is completed by adding beads (0.5 mm, Zymo Research®, USA) and applying vibration with a Tissue Lyser Mill (Qiagen®, USA) for 2 minutes at maximum speed.

Total RNAs are isolated using the Quick-RNA” MiniPrep kit or by extraction with TriReagent. The RNA concentration is measured using a Nanodrop spectrophotometer (Peqlab®, Erlangen, Germany) The samples are either used immediately or stored at −80° C. until use.

Total RNAs of 10,000 to 1,000,000 cells were extracted in 3 replicates from different cultures and different strains of toxic algae. The RNA concentration values obtained were used to obtain an average value of RNA content per cell under optimal culture conditions (FIG. 2). Ayers et al (2005) assume that the exponential growth obtained under optimal culture growth conditions corresponds approximately to what happens during an efflorescence.

c) Embodiment and Synthesis of Nucleic Probes

The probes of the present invention are synthesized according to the methods known to man of the art. They are rehydrated in ultrapure water to obtain a mother solution with a concentration of 100 μM. Three oligonucleotide probes have been designated and tested for the toxic alga Lingulodinium polyedrum (Table 3). The sequence probes SEQ ID NO: 119 and SEQ ID NO: 120 were used to test the position control (PC) and negative control (NC).

TABLE 3
Oligonucleotide probes targeting Lingulodinum polyedrum.

| | Pair of | | | | | | |
|---|---|---|---|---|---|---|---|
| | probes | | | | | | |
| | tested | | | | | | |
| | (SEQ ID | | | GC | | | GC |
| Species | NO:) | Sequence (5′-3′) | Tm | (%) | Sequence (5′-3′) | Tm | (%) |
| PC | 119/120 | GGC AAA CAG | 59.3 | 52 | AGT GCT CTT | 60.1 | 45 |
| (positive | | GAC TGT CAC | | | TTC CAA GAG | | |
| control) | | CCT CAT T | | | GCT TAC ATC TG | | |
| | | (SEQ ID NO: 119) | | | (SEQ ID NO: 120) | | |
| NC | 119/120 | GGC AAA CAG | 59.3 | 52 | AGT GCT CTT | 60.1 | 45 |
| (negative | | GAC TGT CAC | | | TTC CAA GAG | | |
| control) | | CCT CAT T | | | GCT TAC ATC TG | | |
| | | (SEQ ID NO: | | | (SEQ ID NO: 120) | | |
| | | 119) | | | | | |
| Lingulodinium | 119/120 | GGC AAA CAG | 59.3 | 52 | AGT GCT CTT | 60.1 | 45 |
| polyedrum | | GAC TGT CAC | | | TTC CAA GAG | | |
| | | CCT CAT T | | | GCT TAC ATC | | |
| | | (SEQ ID NO: | | | TG | | |
| | | 119) | | | (SEQ ID NO: 120) | | |
| Lingulodinium | 122/123 | GGA CTG TCA | 59.3 | 52 | TTT CCA AGA | 59.5 | 50 |
| polyedrum | | CCC TCA TTA | | | GGC TTA CAT | | |
| | | GTG CTC T | | | CTG CAC CC | | |
| | | (SEQ ID NO: 122) | | | (SEQ ID NO: 123) | | |
| Lingulodinium | 125/126 | CTG CAC CCC | 62.6 | 60 | CCA GAC TAC | 59.3 | 52 |
| polyedrum | | CAT TGG CAA | | | AAC TCA AGG | | |
| | | CGC ATC T | | | CTA CCA G | | |
| | | (SEQ ID NO: 125) | | | (SEQ ID NO: 126) | | |

d) Sandwich Hybridization Test

Probe specificity and sensitivity tests are carried out by sandwich hybridization. The probe captures biotinylated or modified by an amine group (SEQ ID NO: 119; SEQ ID NO: 122; SEQ ID NO: 125) is coupled to a solid support functionalized either by neutravidin or by N-hydroxysulfosuccinimide and the signal probe is coupled to a digoxigenin molecule (SEQ ID NO: 120; SEQ ID NO: 123; SEQ ID NO: 126). The signal probe is placed in the presence of nucleic acid molecules which may contain the target ribosomal nucleic acid complementary to the capture and signal probes. The mixture is placed in the presence of the capture probe which will hybridize to its complementary targets forming a hybrid of three molecules: the capture probe, the signal probe and the target ribosomal nucleic acid. The hybrid complexes are revealed by the digoxigenin attached to the signal probe thanks to a colorimetric reaction initiated by a horseradish peroxidase-type enzyme with its substrate producing a blue colour. The optical density of the colour obtained is measured by a spectrophotometer. The intensity of the colour is proportional to the amount of the target ribosomal nucleic acid present in the sample extract being analysed. Using a calibration curve made with synthetic RNAs, the amount of target nucleic acid is determined. Thanks to a calibration curve carried out with RNA extracted from a known number of target cells in exponential growth phase, the quantity of RNA determined by the first calibration is associated with an estimated number of active live toxic algae cells present in the analysed sample.

The complete test is carried out in less than an hour.

The samples for the sandwich hybridization test are prepared as follows:

Cells from the cultures are collected by filtration on a polycarbonate membrane (porosity 3 μm; Whatman® Nuclepore Track-Etched Membranes). The membranes are transferred to a tube (Eppendorf®) containing 1 mL TriReagent® solution (Sigma®, France) and heated at 65° C. for 10 minutes. They are then subjected to the mill in the presence of 0.5 mm beads (Bashing Beads®, Zymoresearch®) for 1 minute at maximum speed to extract the genetic material. The supernatant is collected and 200 μL of chloroform is added and mixed. The samples are centrifuged for 15 minutes at 4° C. and the aqueous phase is transferred to a clean tube. 0.5 volume of isopropanol is added and the mixture is incubated for 1 hour at −20° C. After 20 minutes centrifugation at 9000 g at 4° C., the supernatant is removed and the pellet is washed twice with 70% ethanol. The pellets are dried in the open air and then solubilised in 50 to 100 μL of ultra pure water. The quantity and quality of RNA obtained is measured by spectrophotometry with NanoDrop (Thermo Scientific®) or NanoVue (Biochrom® Spectrophotometers). The total RNAs are fragmented using a solution comprising 40 mM Trizma base, pH 8.0/100 mM KOAc/30 mM MgOAc for 10 minutes at 65° C. before hybridization.

Hybridization steps are performed in a standard 96-well microplate (Nunc®, Denmark) functionalized with a solution of NeutrAvidin 1 μg/mL, incubated for 24 hours and washed with a saline solution such as PBS 1× (K₂PO₄, 0.1 M; KH₂PO₄, 0.1 M; KCl, 0.1 M; pH 7.6) or with N-hydroxysulfosuccinimide (PolyAn, Germany) The first hybridization step consists of mixing 200 ng of RNA with the hybridization buffer (0.3 M NaCl; 0.08 M Tris-HCl; 0.04% SDS; pH 8) to a final volume of 100 μL containing the signal probe (1 mM). The mixture is heated to 60° C. for 10 minutes. The samples are then cooled and a final 0.05 M EDTA solution is added. The mixture is added to the microplate wells and incubated for 10 minutes at 60° C. The microplate is washed three times with a saline solution such as PBX 1×. 100 μL of anti-DIG-HRP antibody at a concentration of 75 mU/mL is then added and incubated for 15 minutes at room temperature. Then 100 μL of TMB are added and the absorbance is measured after 15 minutes of incubation at a wavelength of 630 nm. The reaction can also be stopped using an acid such as sulphuric acid and the absorbance is measured at a wavelength of 450 nm. Each step of the development is carried out at room temperature with constant agitation and shielded from light.

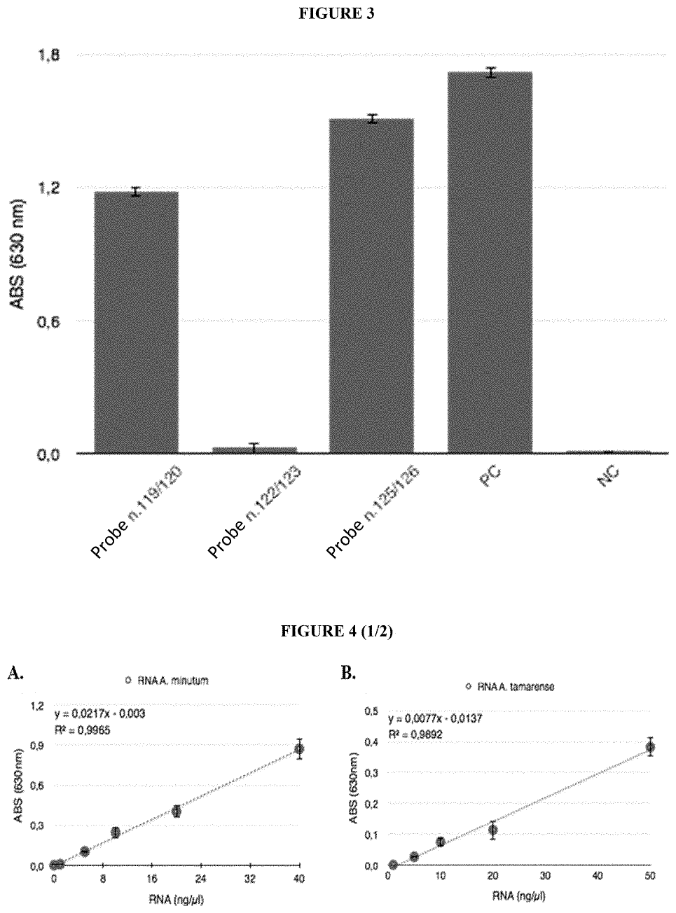

Screening of the following pairs of probes: SEQ ID NO: 119 and SEQ ID NO: 120, SEQ ID NO: 122 and SEQ ID NO: 123 or SEQ ID NO: 125 and SEQ ID NO: 126 was conducted at the same hybridization temperature. The results were compared with those obtained on the positive (PC) and negative (NC) controls (FIG. 3). The positive control is a synthetic DNA fragment of 0.1 μM complementary to both probes and the negative control is a synthetic DNA fragment of 0.1 μM non-complementary to both probes. Probes SEQ ID NO: 119 and SEQ ID NO: 120; and SEQ ID NO: 125 and SEQ ID NO: 126 have been chosen to perform the calibration curves.

e) Calibration Curves to Quantify Target RNAs

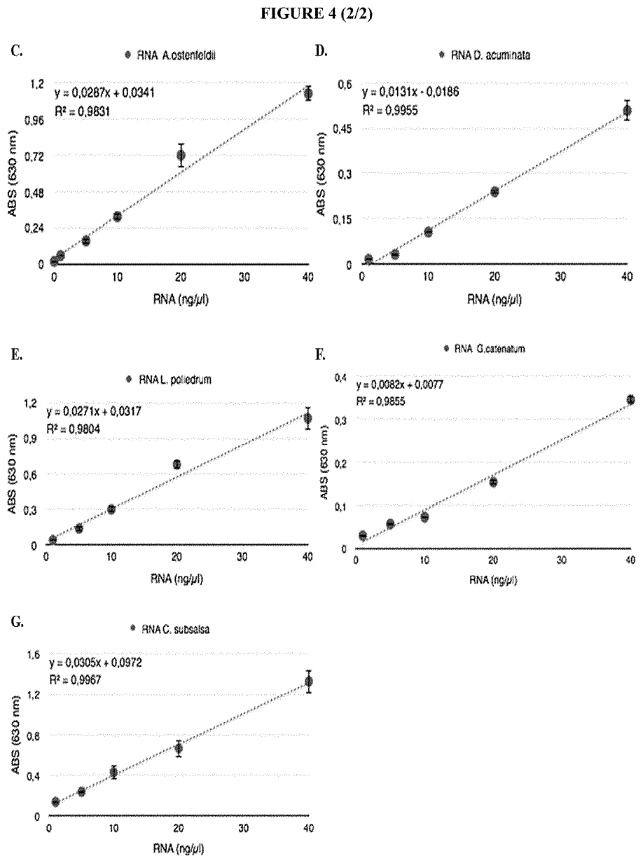

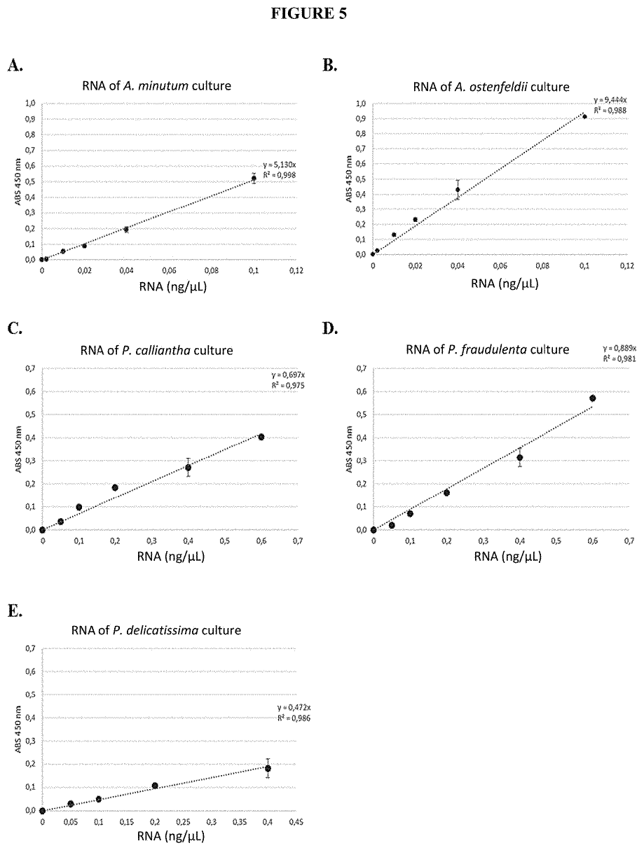

The best combinations of probes were used to establish the calibration curves. These calibration curves measured at 630 nm were developed from RNA extracted from a determined number of algal cells from a non-axial culture, which means bacterial contamination of the culture, as described in parts a) and b) of this example. A dilution of the RNAs was performed to obtain a concentration range of 0.5 ng/μl to 40 ng/μl or 15 to 1200 ng total RNA in a reaction volume of 30 μL. The hybridization steps are performed as in part d) of this example. FIG. 4 shows the calibration curves obtained with serial dilution at an absorbance of 630 nm for the following toxic algae:
A. Alexandrium minutum; probe pair tested: SEQ ID NO: 7 and SEQ ID NO: 8
B. Alexandrium tamarense; pair of probes tested: SEQ ID NO: 17 and SEQ ID NO: 18
C. Alexandrium ostenfeldii; probe pair tested: SEQ ID NO: 11 and SEQ ID NO: 12
D. Dinophysis acuminata; probe pair tested: SEQ ID NO: 40 and SEQ ID NO: 41
E. Lingulodinium polyedrum; pair of probes tested: SEQ ID NO: 119 and SEQ ID NO: 120
F. Gymnodinium catenatum; pair of probes tested: SEQ ID NO: 83 and SEQ ID NO: 84
G. Chattonella subsalsa; pair of probes tested: SEQ ID NO: 74 and SEQ ID NO: 75.

In this calibration curve experiment, the result shows a minimum detection limit between 0.5 and 5 ng/μL of a reaction volume of 30 μL. The total amount of RNA can be related to one litre of culture sample, i.e. between 25 and 250 ng/L.

With the optimised procedure (see g)), the results showed (FIG. 5) a limit of quantification of 0.01 ng/μL, i.e., an amount of 0.5 ng total RNA in 50 μL reaction volume. The detection limit is 0.002 ng/μL reaction volume, i.e., 0.1 ng total RNA in 50 μL reaction volume. The results can be reported per 1 litre of sample, i.e. 0.1 ng/L.

f) Calibration Curves Made from Environmental Samples Supplemented with Cells or Naturally Contaminated Samples and Counted by Microscopy

Calibration curves were performed by adding a known number of cultured cells to an Alexandrium free environmental sample. Cells from non-axial culture of Alexandrium minutum were harvested either in exponential growth phase or in stationary phase as described above FIG. 1. 50 to 5,000 cells were collected in triplicate and filtered on a polycarbonate membrane with a porosity of 3 μm (Whatman® Nuclepore Track-Etched Membranes).

RNA extraction is performed with the QuickRNA® kit (ZymoResearch®, USA) with an elution volume of 60 μL of ultrapure water. The total RNAs are fragmented using a solution comprising 40 mM Trizma base, pH 8.0, 100 mM KOAc and 30 mM MgOAc) for 10 minutes at 65° C. before hybridization.

The hybridization steps are performed in a standard 96-well microplate (Nunc®, Denemark) functionalized with NeutrAvidin solution at 1 μg/mL, incubated for 24 hours with the sequence probe SEQ ID NO: 1 at a concentration of 1 μM. After 24 hours the microplate is washed with a saline solution such as PBS 1× (K₂PO₄, 0.1 M; KH₂PO₄, 0.1 M; KCl, 0.1 M; pH 7.6). The eluate with RNA is mixed with the hybridization buffer (0.3 M NaCl, 0.08 M Tris-HCl, 0.04% SDS, pH 8) to a final volume of 100 μL containing the sequence probe SEQ ID NO: 2 at a concentration of 1 μM. The hybridization mix is heated at 60° C. for 10 minutes, then a final 0.05 M EDTA solution is added. 100 μl of the mixture is dispensed into each well of the microplate and incubated for 10 minutes at 60° C. The microplate is washed three times with a saline solution such as PBX 1×. 100 μL of anti-DIG-HRP antibody at a concentration of 75 mU/mL is then added and incubated for 15 minutes at room temperature. 100 μL of substrate such as TMB is added and absorbance is measured after 15 minutes of reaction at a wavelength of 630 nm.

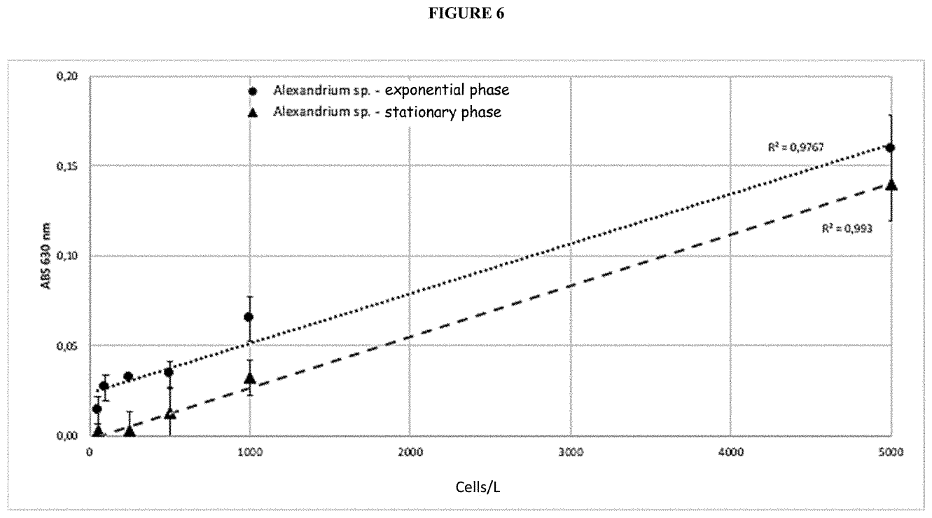

The results (FIG. 6) show an equivalent minimum quantification threshold of 250 and 500 live active cells per litre of sample in the stationary phase and 100 to 250 cells per litre of sample in the exponential phase, corresponding respectively to 5.05 to 10.1 ng total RNA per litre of sample and 2.02 to 5.05 ng total RNA per litre of sample.

As the Dinophysis micro-algae is very difficult to cultivate, the calibration curves are made using naturally contaminated environmental samples. A sample naturally contaminated by Dinophysis was counted by microscopy and between 0 and 150 cells were collected by filtration and used to make the calibration curve. Hybridization of the total RNAs of the natural extract was carried out as described in paragraph d). The absorbance was read at 450 nm after stopping the colorimetric reaction with sulphuric acid.

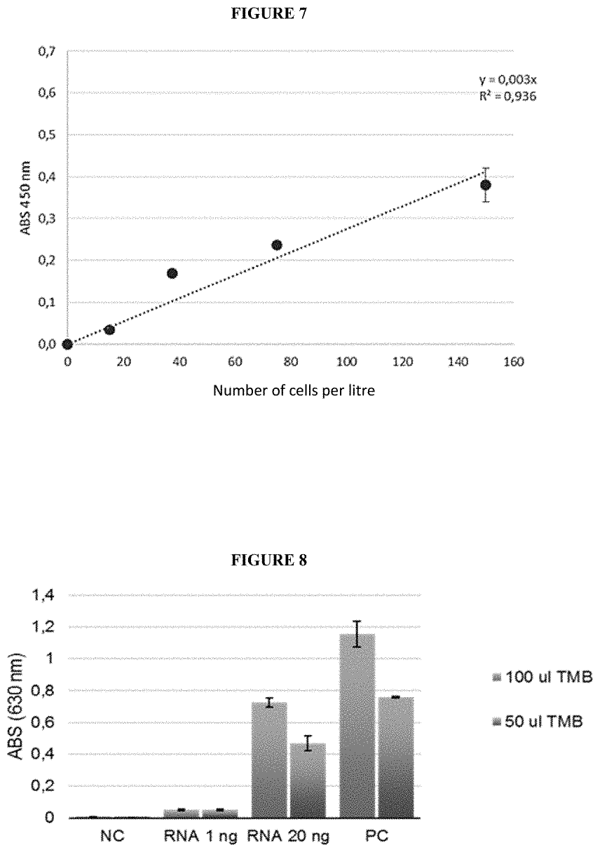

The results (FIG. 7) show, with the optimised procedure, a quantification limit of 5 active living cells estimated per litre on the basis of a signal whose intensity is 10 times that of the standard deviation of the blank, i.e. 0.01 absorbance unit. The detection limit is established at an equivalent of 2 active live cells per litre of natural sample based on a signal whose intensity is 3 times that of the standard deviation of the blank, i.e. 0.003 absorbance units. RNA matching is not optional because the curve is made from contaminated environmental samples whose total RNA content is not equivalent to that obtained from a cell culture.

g) Improved Sensitivity of the Colorimetric Test

Detection and quantification limits have been improved by optimising the conditions for carrying out the procedure by testing different hybridization buffers, different volumes of developing solutions and different wavelengths for reading absorbances.

In a first experiment, two volumes of the development solution are tested. The results indicate an improvement in detection sensitivity (FIG. 8). The results show that the 100 μL volume of TMB solution increases the sensitivity threshold of the procedure by a factor of 1.7 in absorbance units at 630 nm.

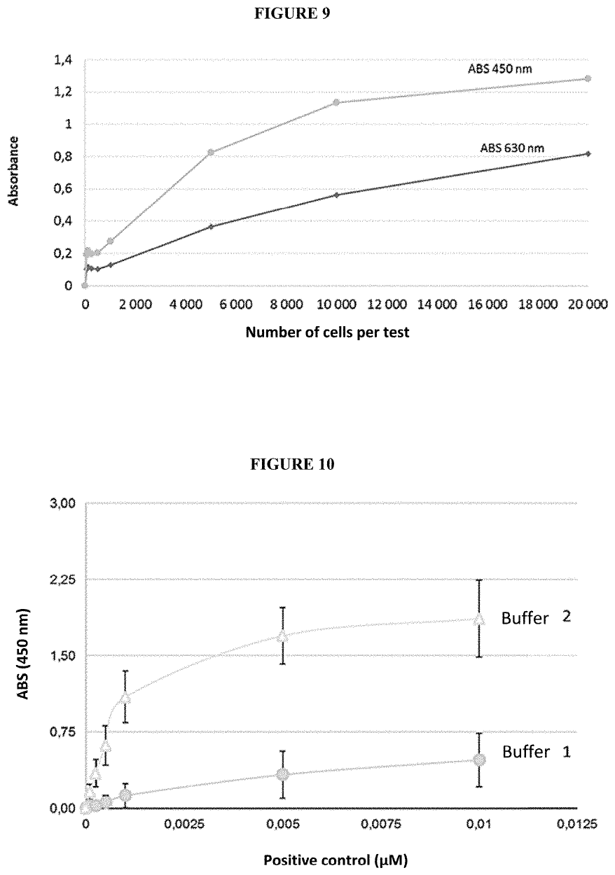

In a second experiment, hybridization tests are carried out in duplicate and the results are measured either at 630 nm or at 450 nm after stopping the revelation with sulphuric acid. The results indicate that the sensitivity is increased by a factor of between 2 and 4 absorbance units using the 450 nm wavelength (FIG. 9).

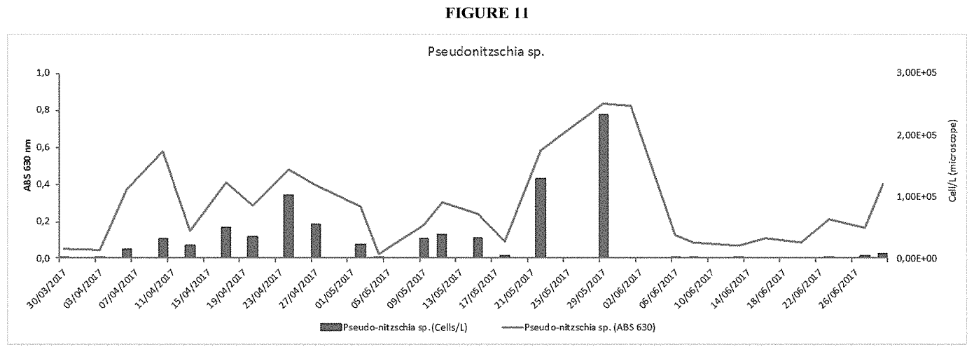

The third experiment consisted in carrying out the hybridization experiments either with hybridization buffer 1 or with hybridization buffer 2 carried out according to the indications described in the said invention. The results are reported in FIG. 10 and show that buffer 2 makes it optional to increase the sensitivity threshold of the procedure by a factor of 2.8 in absorbance units at 450 nm (FIG. 10).

### Example 2: Detection of Algae from Natural Environmental Samples

Implementation comparisons between the present invention and the traditional technique based on the identification and counting of algae by microscopy with the Utermöhl method (1958) have been carried out on natural samples.

a) Detection of Pseudo-nitzschia

The monitoring of Pseudo-nitzschia was carried out in the Thau basin, Bouzigues, South of France from 30 Mar. 2017 to 30 Jun. 2017. Water samples were collected twice a week at a depth of 50 cm. In parallel, a 50 mL sub-sample was collected, fixed to Lugol, and sedimented for 24 hours.

Microscopic counting according to the Utermöhl method required a 12-hour sedimentation step followed by a careful counting of the cells under the microscope. The entire Utermöhl method was carried out in 24 to 48 hours. This method makes it optional to detect the presence or absence of cells of toxic algae of the genus Pseudo-nitzschia and thus to determine the number of cells of toxic algae of the genus Pseudo-nitzschia. However, this method does not determine the activity of cells of toxic algae of the genus Pseudo-nitzschia.

The embodiment of the present invention was carried out in less than one hour and made it optional to determine the activity of the cells of toxic algae of the genus Pseudo-nitzschia as well as the number of cells.

For each hybridization test, 3 litres of seawater are immediately filtered through polycarbonate membranes (porosity 3 μm; Whatman® Nuclepore Track-Etched Membranes). The membranes are transferred to a tube (Eppendorf®) containing 2 mL ZR lysis solution (ZymoResearch®, USA) and heated at 65° C. for 10 minutes. They are then subjected to the mill in the presence of 0.5 mm beads (Bashing Beads®, ZymoResearch®) for 1 minute at maximum speed. RNA extraction is performed with the QuickRNA® kit (ZymoResearch®, USA) with an elution volume of 180 μL of ultrapure water. The total RNAs are fragmented using a solution comprising 40 mM Trizma base, pH 8.0/100 mM KOAc/30 mM MgOAc) for 10 minutes at 65° C. prior to hybridization.

The hybridization steps are performed in a standard 96-well microplate (Nunc®, Denemark) functionalized with NeutrAvidin solution at 1 μg mL-1, incubated for 24 hours with the sequence probe SEQ ID NO: 46 at a concentration of 1 μM. After 24 hours the microplate is washed with a saline solution such as PBS 1× (K₂PO₄, 0.1 M; KH₂PO₄, 0.1 M; KCl, 0.1 M, pH 7.6). The RNA eluate is mixed with the hybridization buffer (0.3 M NaCl, 0.08 M Tris-HCl, 0.04% SDS, pH 8) to a final volume of 300 μl containing the sequence signal probe SEQ ID NO: 47 (1 mM). The hybridization mix is heated at 60° C. for 10 minutes, then a final 0.05 M EDTA solution is added. 100 μl of the mixture is dispensed into 3 wells of the microplate and incubated for 10 minutes at 60° C. The microplate is washed three times with a saline solution such as PBX 1×. 100 μL of anti-DIG-HRP antibody at a concentration of 75 mU/mL is then added and incubated for 15 minutes at room temperature. 100 μL of substrate such as TMB is added and absorbance is measured after 15 minutes of reaction at a wavelength of 630 nm. Each step of the development is carried out at room temperature with constant agitation and shielded from light.

In general, the results obtained by sandwich hybridization tests with naturally contaminated environmental samples are in agreement with the counts obtained by microscopy. However, the present invention makes it optional to obtain the results in less than one hour, compared with 24 to 48 hours for the counting method. The hybridization test has allowed the detection of approximately 500 live active Pseudo-nitzschia cells per litre of sample (FIG. 11).

b) Alexandrium Detection

Alexandrium monitoring was carried out in the Thau Basin, Bouzigues, southern France from 5 Oct. 2017 to 17 Nov. 2017. Water samples were collected twice a week at a depth of 50 cm. In parallel, a 50 mL sub-sample was collected, fixed to Lugol, and sedimented for 24 hours. Microscopic counting according to the Utermöhl method required a 12-hour sedimentation step followed by a careful counting of the cells under the microscope. The entire Utermöhl method was carried out in 24 to 48 hours. This method makes it optional to detect the presence or absence of cells of toxic algae of the genus Alexandrium and thus to determine the number of cells. However, the method does not determine the activity of toxic algal cells of the genus Alexandrium.

The embodiment of the present invention was carried out in less than one hour and made it optional to determine the activity of the cells of toxic algae of the genus Alexandrium as well as the number of cells.

For each hybridization test, 3 litres of environmental water are collected and immediately filtered on polycarbonate membranes (porosity 5 μm; Whatman® Nuclepore Track-Etched Membranes). The membranes are transferred to a tube (Eppendorf®) containing 2 mL of ZR lysis solution (ZymoResearch®, USA) and heated at 60° C. for 10 minutes. They are then subjected to the mill in the presence of 0.5 mm beads (Bashing Beads®, ZymoResearch®) for 2 minutes at maximum speed. RNA extraction is performed with the QuickRNA® kit (ZymoResearch®, USA) with an elution volume of 180 μl of ultrapure water, i.e. 60 μL per replica. The total RNAs are fragmented using a solution comprising 40 mM Trizma base, pH 8.0/100 mM KOAc/30 mM MgOAc) for 10 minutes at 65° C. before hybridization and the samples are stabilized with a 0.05% EDTA solution.

The hybridization steps are performed in a standard 96-well microplate (Nunc®, Denemark) functionalized with NeutrAvidin solution at 1 μg/mL, incubated for 24 hours with the sequence probe SEQ ID NO: 1 at a concentration of 1 μM. After 24 hours the microplate is washed with a saline solution such as PBS 1× (K₂PO₄, 0.1 M; KH₂PO₄, 0.1 M; KCl, 0.1 M; pH 7.6). The RNA eluate is mixed with the hybridization buffer (0.3 M NaCl, 0.08 M Tris-HCl, 0.04% SDS, pH 8) to a final volume of 300 μl containing the sequence probe SEQ ID NO: 2 at a concentration of 1 μM. The hybridization mix is heated at 60° C. for 10 minutes, then a final 0.05 M EDTA solution is added. 100 μl of the mixture is dispensed into 3 wells of the microplate and incubated for 15 minutes at 60° C. The microplate is washed three times with a saline solution such as PBS 1×. 100 μL of anti-DIG-HRP antibody at a concentration of 75 mU/mL is then added and incubated for 15 minutes at room temperature. 100 μL of substrate such as TMB is added and absorbance is measured after 15 minutes of reaction at a wavelength of 630 nm. Each stage of the development is carried out at room temperature with constant agitation and away from light.

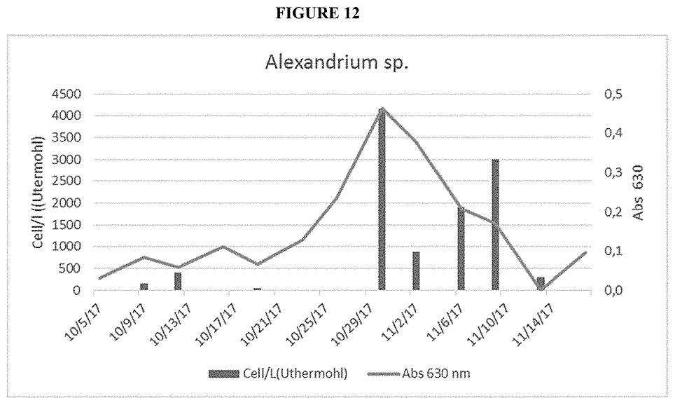

In general, the results obtained by sandwich hybridization tests with naturally contaminated environmental samples are in agreement with the counts obtained by microscopy. However, the present invention makes it optional to obtain the results in less than one hour, compared with 24 to 48 hours for the counting method. The hybridization test has allowed the detection of approximately 160 live active Alexandrium cells per litre of sample (FIG. 12).

c) Detection of Dinophysis sp.

Dinophysis monitoring was carried out in the Leucate lagoon in southern France from 15 Oct. 2018 to 30 Mar. 2019. Water samples were collected twice a week at a depth of 50 cm. In parallel, a 50 mL sub-sample was collected, fixed to Lugol, and sedimented for 24 hours. Microscopic counting according to the Utermöhl method required a 12-hour sedimentation step followed by a careful counting of the cells under the microscope. The entire Utermöhl method was carried out in 24 to 48 hours. The method did not determine the activity but only the presence or absence and number of cells. The embodiment of the present invention was carried out in less than one hour and made it optional to determine the activity and the number of cells in comparison with the results obtained a posteriori by the Utermöhl method.

For each hybridization test, 3 litres of water are immediately filtered through polycarbonate membranes (5 μm porosity; Whatman® Nuclepore Track-Etched Membranes). The membranes are transferred to a tube (Eppendorf®) containing 2 mL ZR lysis solution (ZymoResearch®, USA) and heated at 60° C. for 10 minutes. They are then subjected to the mill in the presence of 0.5 mm beads (Bashing Beads®, ZymoResearch®) for 2 minutes at maximum speed. RNA extraction is performed with the QuickRNA® kit (ZymoResearch®, USA) with an elution volume of 180 μl of ultrapure water. The total RNAs are fragmented using a solution comprising 40 mM Trizma base, pH 8.0/100 mM KOAc/30 mM MgOAc) for 10 minutes at 65° C. prior to hybridization and the samples are stabilized with a 0.05% EDTA solution.

The hybridization steps are performed in a standard 96-well microplate (Nunc®, Denemark) functionalized with NeutrAvidine solution at 1 μg/mL, incubated for 24 hours with the n-probe. 1 (SEQ ID NO: 1) at a concentration of 1 μM. After 24 hours the microplate is washed with a saline solution such as PBS 1×(K₂PO₄, 0.1 M; KH₂PO₄, 0.1 M; KCl, 0.1 M; pH 7.6). The RNA eluate is mixed with the hybridization buffer (0.3 M NaCl, 0.08 M Tris-HCl, 0.04% SDS, pH 8) to a final volume of 300 μl containing the n-probe. 2 ‘SEQ ID NO: 2) at a concentration of 1 μM. The hybridization mix is heated at 60° C. for 10 minutes and then a final 0.05 M EDTA solution is added. 100 μl of the mixture is dispensed into 3 wells of the microplate and incubated for 15 minutes at 60° C. The microplate is washed three times with a saline solution such as PBS 1×. 100 μL of anti-DIG-HRP antibody at a concentration of 75 mU/mL is then added and incubated for 15 minutes at room temperature. 100 μL of substrate such as TMB is added and the absorbance is measured after 15 minutes of reaction and after stopping the reaction with sulphuric acid at a wavelength of 450 nm. Each step of the development is carried out at room temperature with constant agitation and shielded from light.

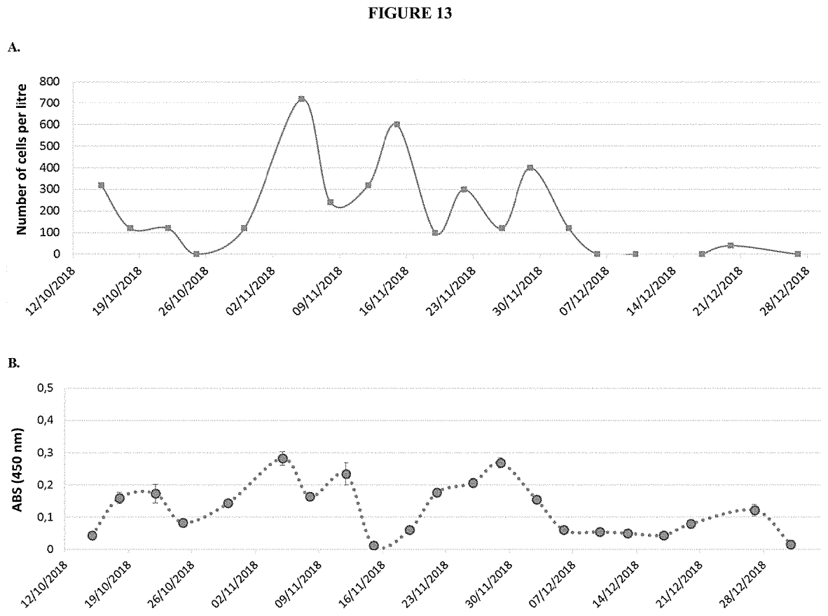

In general, the absorbance results obtained by sandwich hybridization tests with naturally contaminated environmental samples are in agreement with the counts obtained by microscopy. The hybridization test detected less than 40 active live cells of Dinophysis sp. per litre based on microscopic counts (FIG. 13).

### REFERENCES

Ayers, K., Rhodes, L. L., Tyrrell, J., Gladstone, M., Scholin, C., 2005. International accreditation of sandwich hybridization assay format DNA probes for microalgae. N. Z. J. Mar. Freshwater Res. 39, 1225-1231.
Daniel. MacDougall, Warren B. Crummett et al. ACS (1980) Guidelines for Data Acquisition and Data Quality Evaluation in Environmental Chemistry, Analytical chemistry, 52, 14, 2242-2249
Guillard, R. R. L., Hargraves, P. E., (1993). Stichochrysis immobilisis a diatom, not a chrysophyte. Phycologia 32, 234-236
Guillard, R. R. L. and Ryther, J. H. (1962). Studies of marine planktonic diatoms. I. Cyclotella nana Hustedt and Detonula confervacea Cleve. Can. J. Microbiol. 8: 229-239
J V Tyrrell, P R Bergquist, P L Bergquist, C A Scholin. Compositions and methods for detecting raphidophytes, U.S. Pat. No. 6,787,648
Sonja Diercks, Katja Metfies, Linda K., Molecular probe sets for the detection of toxic algae for use in sandwich hybridization formats. Journal of Plankton Research, Vol. 30, Issue 4, 1 Apr. 2008, Pages 439-448
Sonja Diercks, Linda K Medlin, Katja Metfies. Colorimetric detection of the toxic Dinoflagellate Alexandrium minutum using sandwich hybridization in a microtiter plate assay—Harmful Algae, Vol. 7, Issue 2, February 2008, Pages 137-145
Joe D. Taylor, Jessica U. Kegel, Jane M. Lewis, Linda K. Medlin. Validation of the detection of Alexandrium species using specific RNA probes tested in a microarray format: Calibration of signal using variability of RNA content with environmental conditions. Harmful Algae, Vol. 37, 2014, Pages 17-27
Utermohl H., (1958), Zur Vervollkommnung der quantitativen Phytoplankton-Methodik: Mit 1 Tabelle und 15 abbildungen im Text und auf 1 Tafel, International Association for Theoretical and Applied Limnology: Mitteilungen, 9 (1), p 1-38
Yuji Tanaka and Makoto Tsuneoka (2018), Control of Ribosomal RNA Transcription by Nutrients. Chapitre 2, Pages 25-51 de “Gene expression and regulation in mammalian cells: Transcription towards the establishment of novel therapeutics.”

1-23. (canceled)

24. A method for detecting active living cells of toxic algae in a sample likely to contain at least one toxic alga of the genus Dinophysis and/or Alexandrium using of at least one pair of probes specific to toxic algae of the genus Dinophysis or Alexandrium, the sequences of said probes being chosen from x elements of one of the following sets:
(SEQ ID NO: 1, SEQ ID NO: 2 or SEQ ID NO: 3)
(SEQ ID NO: 4 and SEQ ID NO: 5)
(SEQ ID NO: 6 and SEQ ID NO: 7)
(SEQ ID NO: 8, SEQ ID NO: 9 or SEQ ID NO: 10)
(SEQ ID NO: 11, SEQ ID NO: 12 or SEQ ID NO: 13)
(SEQ ID NO: 14, SEQ ID NO: 15 or SEQ ID NO: 16)
(SEQ ID NO: 17, SEQ ID NO: 18 or SEQ ID NO: 19)
(SEQ ID NO: 20, SEQ ID NO: 21 or SEQ ID NO: 22)
(SEQ ID NO: 23, SEQ ID NO: 24 or SEQ ID NO: 25)
(SEQ ID NO: 26, SEQ ID NO: 27 or SEQ ID NO: 28)
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44 or SEQ ID NO: 45), or
(SEQ ID NO: 46, SEQ ID NO: 47 or SEQ ID NO: 48)
x being 2 or 3,
or the sequences of said probes having at least 92% identity with the abovementioned sequences SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47 or SEQ ID NO: 48,
one probe of said pair being a capture probe linked to at least one attachment molecule positioned at 3′ or 5′ of its sequence and the other probe of said pair being a signal probe linked to at least one marking molecule positioned at 3′ or 5′ of its sequence,
said capture probe and said signal probe being capable of hybridizing with the ribosomal nucleic acid of a toxic alga of the genus Dinophysis or Alexandrium optionally present in said sample to form a complex,
the minimum detection threshold of the toxic algae of the genus Dinophysis or Alexandrium being
from 100 to 500 active living cells per litre of sample and in particular less than 200 active living cells per litre of sample, or
less than or equal to 0.10 ng RNA per litre of sample and in particular including 0.01 to 0.09 ng RNA per litre of sample.

25. The method according to claim 24, wherein the sequences of the said probes being as follows:
(SEQ ID NO: 29 and SEQ ID NO: 30), (SEQ ID NO: 29 and SEQ ID NO: 31), (SEQ ID NO: 30 and SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40 and SEQ ID NO: 41), (SEQ ID NO: 40 and SEQ ID NO: 42), (SEQ ID NO: 41 and SEQ ID NO: 42)
(SEQ ID NO: 43 and SEQ ID NO: 44), (SEQ ID NO: 43 and SEQ ID NO: 45), (SEQ ID NO: 44 and SEQ ID NO: 45)
(SEQ ID NO: 46 and SEQ ID NO: 47), (SEQ ID NO: 46 and SEQ ID NO: 48), (SEQ ID NO: 47 and SEQ ID NO: 48.

26. The method according to claim 24, wherein the sequences of the said probes being as follows:
(SEQ ID NO: 1 and SEQ ID NO: 2), (SEQ ID NO: 1 and SEQ ID NO: 3), (SEQ ID NO: 2 and SEQ ID NO: 3)
(SEQ ID NO: 4 and SEQ ID NO: 5)
(SEQ ID NO: 6 and SEQ ID NO: 7)
(SEQ ID NO: 8 and SEQ ID NO: 9), (SEQ ID NO: 8 and SEQ ID NO: 10), (SEQ ID NO: 9 and SEQ ID NO: 10)
(SEQ ID NO: 11 and SEQ ID NO: 12), (SEQ ID NO: 11 and SEQ ID NO: 13), (SEQ ID NO: 12 and SEQ ID NO: 13)
(SEQ ID NO: 14 and SEQ ID NO: 15), (SEQ ID NO: 14 and SEQ ID NO: 16), (SEQ ID NO: 15 and SEQ ID NO: 16)
(SEQ ID NO: 17 and SEQ ID NO: 18), (SEQ ID NO: 17 and 19), (SEQ ID NO: 18 and SEQ ID NO: 19)
(SEQ ID NO: 20 and SEQ ID NO: 21), (SEQ ID NO: 20 and SEQ ID NO: 22), (SEQ ID NO: 21 and SEQ ID NO: 22), or
(SEQ ID NO: 23 and SEQ ID NO: 24), (SEQ ID NO: 23 and SEQ ID NO: 25), (SEQ ID NO: 24 and SEQ ID NO: 25)
(SEQ ID NO: 26 and SEQ ID NO: 27), (SEQ ID NO: 26 and SEQ ID NO: 28), (SEQ ID NO: 27 and SEQ ID NO: 28).

27. The method according to claim 24 for detecting active living cells of toxic algae in a sample likely to contain at least one toxic alga of the genus Dinophysis and/or Alexandrium using of at least one pair of probes specific to toxic algae of the genus Dinophysis and at least one pair of probes specific to toxic algae of the genus Alexandrium.

28. The method according to claim 24, in which
said capture probe is linked to at least one attachment molecule positioned at 5′ of its sequence and said signal probe is linked to at least one marker molecule positioned 5′ of its sequence, or
said capture probe is linked to at least one attachment molecule positioned at 5′ of its sequence and said signal probe is linked to at least one marker molecule positioned 3′ of its sequence, or
said capture probe is linked to at least one attachment molecule positioned at 3′ of its sequence and said signal probe is linked to at least one marker molecule positioned at 5′ of its sequence, or
said capture probe is linked to at least one attachment molecule positioned at 3′ of its sequence and said signal probe is linked to at least one labelling molecule positioned at 3′ of its sequence,
said “at least one attachment molecule” being in particular selected from a biotin, avidin, streptavidin molecule, a thiol group, an amine group and a carbon, preferably a biotin molecule,
said “at least one labelling molecule” is chosen in particular from a fluorochrome, a biotin, a biotin-bound molecule, digoxigenin, an enzyme using a chemiluminescent substrate, an enzyme using a chromogenic substrate or an enzyme using an electrochemically oxidised substrate, preferably digoxigenin.

29. The method according to claim 24, in which
said capture probe is linked to at least one attachment molecule positioned at 5′ of its sequence and said signal probe is linked to at least one marker molecule positioned 5′ of its sequence, or
said capture probe is linked to at least one attachment molecule positioned at 5′ of its sequence and said signal probe is linked to at least one marker molecule positioned 3′ of its sequence, or
said capture probe is linked to at least one attachment molecule positioned at 3′ of its sequence and said signal probe is linked to at least one marker molecule positioned at 5′ of its sequence, or
said capture probe is linked to at least one attachment molecule positioned at 3′ of its sequence and said signal probe is linked to at least one labelling molecule positioned at 3′ of its sequence,
said “at least one attachment molecule” being in particular selected from a biotin, avidin, streptavidin molecule, a thiol group, an amine group and a carbon, preferably a biotin molecule,
said “at least one labelling molecule” is chosen in particular from a fluorochrome, a biotin, a biotin-bound molecule, digoxigenin, an enzyme using a chemiluminescent substrate, an enzyme using a chromogenic substrate or an enzyme using an electrochemically oxidised substrate, preferably digoxigenin;
and wherein:
said enzyme using a chromogenic substrate is alkaline phosphatase and said chromogenic substrate is tetrazolium nitroblue (NBT) or bromochlorylindolophosphate (BCIP), or
said enzyme using a chromogenic substrate is horseradish peroxidase (HRP) and said chromogenic substrate is selected from 3,3′-Diaminobenzidine (DAB), 3,3′,5,5′-Tetramethylbenzidine (TMB), or 2,2′-azino-bis(3-ethylbenzothiazoline-6-sulphonic acid) (ABTS).

30. A pair of probes for the detection of active living cells of toxic algae whose sequences are selected from x elements of one of the following sets:
(SEQ ID NO: 1, SEQ ID NO: 2 or SEQ ID NO: 3)
(SEQ ID NO: 4 and SEQ ID NO: 5)
(SEQ ID NO: 6 and SEQ ID NO: 7)
(SEQ ID NO: 8, SEQ ID NO: 9 or SEQ ID NO: 10)
(SEQ ID NO: 11, SEQ ID NO: 12 or SEQ ID NO: 13)
(SEQ ID NO: 14, SEQ ID NO: 15 or SEQ ID NO: 16)
(SEQ ID NO: 17, SEQ ID NO: 18 or SEQ ID NO: 19)
(SEQ ID NO: 20, SEQ ID NO: 21 or SEQ ID NO: 22),
(SEQ ID NO: 23, SEQ ID NO: 24 or SEQ ID NO: 25),
(SEQ ID NO: 26, SEQ ID NO: 27 or SEQ ID NO: 28)
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44 or SEQ ID NO: 45), or
(SEQ ID NO: 46, SEQ ID NO: 47 or SEQ ID NO: 48)
x being 2 or 3
or whose sequences have at least 92% identity with the above-mentioned sequences
SEQ ID NO: 1 to SEQ ID NO: 48.

31. The pair of probes according to claim 30 for the detection of active living cells of toxic algae of the genus Dinophysis whose sequences are selected from x elements of one of the following sets:
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44 or SEQ ID NO: 45), or
(SEQ ID NO: 46, SEQ ID NO: 47 or SEQ ID NO: 48)
x being 2 or 3,
or the sequences of said probes having at least 92% identity with the abovementioned sequences SEQ ID NO: 29 to SEQ ID NO: 48.

32. The pair of probes according to claim 30 for the detection of active living cells of toxic algae of the genus Dinophysis, the sequences of the probes of the said pairs being as follows:
(SEQ ID NO: 29 and SEQ ID NO: 30), (SEQ ID NO: 29 and SEQ ID NO: 31), (SEQ ID NO: 30 and SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40 and SEQ ID NO: 41), (SEQ ID NO: 40 and SEQ ID NO: 42), (SEQ ID NO: 41 and SEQ ID NO: 42)
(SEQ ID NO: 43 and SEQ ID NO: 44), (SEQ ID NO: 43 and SEQ ID NO: 45), (SEQ ID NO: 44 and SEQ ID NO: 45)
(SEQ ID NO: 46 and SEQ ID NO: 47), (SEQ ID NO: 46 and SEQ ID NO: 48), (SEQ ID NO: 47 and SEQ ID NO: 48.

33. The pair of probes according to claim 30 for the detection of active living cells of toxic algae of the genus Alexandrium whose sequences are selected from x elements of one of the following sets:
(SEQ ID NO: 1, SEQ ID NO: 2 or SEQ ID NO: 3)
(SEQ ID NO: 4 and SEQ ID NO: 5)
(SEQ ID NO: 6 and SEQ ID NO: 7)
(SEQ ID NO: 8, SEQ ID NO: 9 or SEQ ID NO: 10)
(SEQ ID NO: 11, SEQ ID NO: 12 or SEQ ID NO: 13)
(SEQ ID NO: 14, SEQ ID NO: 15 or SEQ ID NO: 16)
(SEQ ID NO: 17, SEQ ID NO: 18 or SEQ ID NO: 19)
(SEQ ID NO: 20, SEQ ID NO: 21 or SEQ ID NO: 22),
(SEQ ID NO: 23, SEQ ID NO: 24 or SEQ ID NO: 25),
(SEQ ID NO: 26, SEQ ID NO: 27 or SEQ ID NO: 28)
x being 2 or 3
or whose sequences have at least 92% identity with the above-mentioned sequences SEQ ID NO: 1 to SEQ ID NO: 28.

34. The pair of probes according to claim 30 for the detection of active living cells of toxic algae of the genus Alexandrium, the sequences of the probes of the said pairs being as follows:
(SEQ ID NO: 1 and SEQ ID NO: 2), (SEQ ID NO: 1 and SEQ ID NO: 3), (SEQ ID NO: 2 and SEQ ID NO: 3)
(SEQ ID NO: 4 and SEQ ID NO: 5)
(SEQ ID NO: 6 and SEQ ID NO: 7)
(SEQ ID NO: 8 and SEQ ID NO: 9), (SEQ ID NO: 8 and SEQ ID NO: 10), (SEQ ID NO: 9 and SEQ ID NO: 10)
(SEQ ID NO: 11 and SEQ ID NO: 12), (SEQ ID NO: 11 and SEQ ID NO: 13), (SEQ ID NO: 12 and SEQ ID NO: 13)
(SEQ ID NO: 14 and SEQ ID NO: 15), (SEQ ID NO: 14 and SEQ ID NO: 16), (SEQ ID NO: 15 and SEQ ID NO: 16)
(SEQ ID NO: 17 and SEQ ID NO: 18), (SEQ ID NO: 17 and 19), (SEQ ID NO: 18 and SEQ ID NO: 19)
(SEQ ID NO: 20 and SEQ ID NO: 21), (SEQ ID NO: 20 and SEQ ID NO: 22), (SEQ ID NO: 21 and SEQ ID NO: 22), or
(SEQ ID NO: 23 and SEQ ID NO: 24), (SEQ ID NO: 23 and SEQ ID NO: 25), (SEQ ID NO: 24 and SEQ ID NO: 25)
(SEQ ID NO: 26 and SEQ ID NO: 27), (SEQ ID NO: 26 and SEQ ID NO: 28), (SEQ ID NO: 27 and SEQ ID NO: 28).

35. A process for detecting active living cells of toxic algae in a sample likely to contain at least one toxic alga of the genus Dinophysis and/or Alexandrium comprising the following steps:
a) preparation of the said sample to be analysed in order to obtain a prepared sample,
b) optional hybridization resulting from the contact of the said prepared sample with a capture probe and a signal probe specific to toxic algae of the genus Dinophysis or Alexandrium, the capture probe and the signal probe forming a pair of probes, the sequences of the said pair of probes being chosen from x elements of one of the following sets:
(SEQ ID NO: 1, SEQ ID NO: 2 or SEQ ID NO: 3)
(SEQ ID NO: 4 and SEQ ID NO: 5)
(SEQ ID NO: 6 and SEQ ID NO: 7)
(SEQ ID NO: 8, SEQ ID NO: 9 or SEQ ID NO: 10)
(SEQ ID NO: 11, SEQ ID NO: 12 or SEQ ID NO: 13)
(SEQ ID NO: 14, SEQ ID NO: 15 or SEQ ID NO: 16)
(SEQ ID NO: 17, SEQ ID NO: 18 or SEQ ID NO: 19)
(SEQ ID NO: 20, SEQ ID NO: 21 or SEQ ID NO: 22)
(SEQ ID NO: 23, SEQ ID NO: 24 or SEQ ID NO: 25)
(SEQ ID NO: 26, SEQ ID NO: 27 or SEQ ID NO: 28)
(SEQ ID NO: 29, SEQ ID NO: 30 or SEQ ID NO: 31)
(SEQ ID NO: 32 and SEQ ID NO: 33)
(SEQ ID NO: 34 and SEQ ID NO: 35)
(SEQ ID NO: 36 and SEQ ID NO: 37)
(SEQ ID NO: 38 and SEQ ID NO: 39)
(SEQ ID NO: 40, SEQ ID NO: 41 or SEQ ID NO: 42)
(SEQ ID NO: 43, SEQ ID NO: 44), or
(SEQ ID NO: 46, SEQ ID NO: 47 or SEQ ID NO: 48)
x being 2 or 3,
or the sequences of said probes having at least 92% identity with the abovementioned sequences SEQ ID NO: 1 to SEQ ID NO: 48,
said capture probe and said signal probe being capable of hybridizing with the ribosomal nucleic acid of a toxic alga of the genus Dinophysis or Alexandrium optionally present in said sample to form a complex,
c) detection of said optional complex,
d) quantification of toxic algae of the genus Dinophysis or Alexandrium, in the case of hybridization,
hybridization indicating the presence of toxic algae of the genus Dinophysis or Alexandrium,
the minimum detection threshold of the toxic algae of the genus Dinophysis or Alexandrium being
from 100 to 500 active living cells per litre of sample (cells/L) and in particular less than 200 active living cells per litre of sample (cells/L), or
less than or equal to 0.10 ng RNA per litre of sample and in particular from 0.01 to 0.09 ng RNA per litre of sample,
the duration of the implementation of steps b) and c) being less than one hour.

36. The process according to claim 35 for detecting active living cells of toxic algae in a sample likely to contain at least one toxic alga of the genus Dinophysis and/or Alexandrium comprising the contact of the said prepared sample with a capture probe and a signal probe specific to toxic algae of the genus Dinophysis, and the contact of the said prepared sample with a capture probe and a signal probe specific to toxic algae of the genus Alexandrium.

37. The process of detection according to claim 35 wherein
said capture probe is linked to at least one attachment molecule positioned at 5′ to its sequence and said signal probe is linked to at least one marker molecule positioned at 5′ to its sequence, or
said capture probe is linked to at least one attachment molecule positioned at 5′ of its sequence and said signal probe is linked to at least one marker molecule positioned at 3′ of its sequence, or
said capture probe is linked to at least one attachment molecule positioned at 3′ of its sequence and said signal probe is linked to at least one marker molecule positioned at 5′ of its sequence, or
said capture probe is linked to at least one attachment molecule positioned at 3′ of its sequence and said signal probe is linked to at least one labelling molecule positioned at 3′ of its sequence,
said “at least one attachment molecule” being in particular selected from a biotin, avidin, streptavidin molecule, a thiol group, an amine group and a carbon group, preferably a biotin molecule,
said “at least one marking molecule” being chosen in particular from a fluorochrome, a biotin, a biotin-bound molecule, digoxigenin, an enzyme using a chemiluminescent substrate, an enzyme using a chromogenic substrate or an enzyme using an electrochemically oxidised substrate, preferably digoxigenin.

38. The process of detection according to claim 35 wherein:
said capture probe is linked to at least one attachment molecule positioned at 5′ to its sequence and said signal probe is linked to at least one marker molecule positioned at 5′ to its sequence, or
said capture probe is linked to at least one attachment molecule positioned at 5′ of its sequence and said signal probe is linked to at least one marker molecule positioned at 3′ of its sequence, or
said capture probe is linked to at least one attachment molecule positioned at 3′ of its sequence and said signal probe is linked to at least one marker molecule positioned at 5′ of its sequence, or
said capture probe is linked to at least one attachment molecule positioned at 3′ of its sequence and said signal probe is linked to at least one labelling molecule positioned at 3′ of its sequence,
said “at least one attachment molecule” being in particular selected from a biotin, avidin, streptavidin molecule, a thiol group, an amine group and a carbon group, preferably a biotin molecule,
said “at least one marking molecule” being chosen in particular from a fluorochrome, a biotin, a biotin-bound molecule, digoxigenin, an enzyme using a chemiluminescent substrate, an enzyme using a chromogenic substrate or an enzyme using an electrochemically oxidised substrate, preferably digoxigenin;
and wherein:
said enzyme using a chromogenic substrate is alkaline phosphatase and said chromogenic substrate is tetrazolium nitroblue (NBT) or bromochlorylindolophosphate (BCIP), or
said enzyme using a chromogenic substrate is horseradish peroxidase (HRP) and said chromogenic substrate is selected from 3,3′-Diaminobenzidine (DAB), 3,3′,5,5′-Tetramethylbenzidine (TMB), or 2,2′-azino-bis(acid 3-ethylbenzothiazoline-6-sulphonic) (ABTS).
